(19) United States
(12) Patent Application Publication
(10) Pub. No.: US 2010/0298384 A1
(43) Pub. Date: Nov. 25, 2010

(54) NOVEL OXAZOLIDINONE COMPOUNDS AS ANTIINFECTIVE AGENTS

(76) Inventors: Mohamed Takhi, Hyderabad (IN); Jagattaran Das, Hyderabad (IN); Javed Iqbal, Hyderabad (IN); Natesan Selvakumar, Hyderabad (IN); Sreenivas Kandepu, Hyderabad (IN); M. Sitaram Kumar, Hyderabad (IN)

Correspondence Address: Nelson Mullins Riley & Scarborough LLP;IP Department, 100 North Tryon Street, 42nd Floor, Charlotte, NC, 28202-4000, US

(21) Appl. No.: 12/517,280

(22) PCT Filed: Dec. 4, 2007
(86) PCT No.: PCT/US07/24843

§ 371 Date: Jul. 7, 2010

Related U.S. Application Data
(60) Provisional application No. 60/872,640, filed on Dec. 4, 2006.

(51) Int. Cl. A61K 31/4439; C07D 263/30; C07D 277/28; C07D 413/14; A61K 31/422; A61K 31/427; A61P 31/04

(52) U.S. Cl. 514/340; 548/235; 548/205; 546/269.1; 514/374; 514/365

(57) ABSTRACT

The present invention relates to novel oxazolidinone compounds of formula (I) with antibacterial activity, their pharmaceutically acceptable salts, their stereoisomers, their prodrugs, pharmaceutical compositions comprising the same and to their use as therapeutic agents

### CROSS-REFERENCE TO RELATED APPLICATIONS

The present application is a Patent Cooperation Treaty application and claims the benefit of U.S. Provisional Application No. 60/872,640, filed Dec. 4, 2006, which is relied on herein and incorporated herein by reference in its entirety.

### FIELD OF THE INVENTION

The present invention relates to novel oxazolidinone compounds with antibacterial activity, their stereoisomers, their prodrugs, their pharmaceutically acceptable salts thereof. The present invention also provides, pharmaceutical compositions comprising the compound of formula (I) and their use as therapeutic agents.

### BACKGROUND OF THE INVENTION

In general, bacterial pathogens may be classified as either Gram-positive or Gram-negative pathogens. Antibiotic compounds with effective activity against both Gram-positive and Gram-negative pathogens are generally regarded as having a broad spectrum of activity.

Gram-positive pathogens, for example, Staphylococci, Enterococci, Streptococci and Mycobacteria, are of interest because of the development of resistant strains, which are both difficult to treat and difficult to eradicate from the hospital environment once established. Examples of these resistant strains are methicillin resistant staphylococcus (MRS), methicillin resistant coagulase negative staphylococci (MRCNS), penicillin resistant Streptococcus pneumoniae (PRSP), and multi-drug resistant Enterococcus faecium (MREF). Further, resistant strains of Gram-negative such as H. influenzae and M. catarrhalis have been identified (F. D. Lowry, “Antimicrobial Resistance: The Example of Staphylococcus aureus,” Clin. Invest., 2003 111(9), 1265-1273).

The escalation of resistance to antibiotics once useful for treatment of bacterial infections resulting from such pathogens is problematic in the United States and Europe (Drugs Exp. Clin. Res. 1994, XX, 215-224; Am. J. Surg. 1995, 5A (Suppl.), 8S-12S; Drugs, 1994, 48, 678-688; and Current Pharmaceutical Design, 1996, Vol. 2, No. 2, pp 175-194). At present, the major clinical effective antibiotic for treatment of such resistant Gram-positive pathogens is vancomycin, a glycopeptide. However, antibacterial resistance to vancomycin and other glycopeptides is also emerging and escalating. Thus, the development of new synthetic and semi-synthetic antibacterial compounds effective against resistant bacteria is the subject of constant current research.

One class of synthetic compounds that have been developed is the oxazolidinone compounds, exemplified by eperezoid and linezolid, which constitute a class of orally-active, synthetic antibacterial agents. Oxazolidinones are a new class of synthetic antimicrobial agents which kill gram positive pathogens by inhibiting a very early stage of protein synthesis. Oxazolidinones inhibit the formation of the ribosomal initiation complex involving 30S and 50S ribosomes leading to the prevention of initiation complex formation. Due to their mechanism of action, these compounds are active against pathogens resistant to other clinically useful antibiotics.

WO 95/07271 (Barbachyn et al.) describes oxazine and thiazine oxazolidinone derivatives such as linezolid and its analog, which are useful antimicrobial agents and are effective against a number of human and veterinary pathogens, including gram-positive aerobic bacteria such as multiple drug-resistant staphylococci, streptococci and enterococci as well as anaerobic organisms such as Bacteroides spp. and Clostridia spp. species, and acid-fast organisms such as Mycobacterium tuberculosis, Mycobacterium avium and Mycobacterium spp.

U.S. Pat. No. 5,792,765 to Riedl et al. describes a series of substituted oxazolidinones (cyanoguanidine, cyanoamidines, and amidines) useful as antibacterial medicaments. U.S. Pat. No. 5,910,504 to Hutchinson describes a series of heteroaromatic ring substituted phenyl oxazolidinones. WO 98/54161 (Hester et al.) describes amides, thioamides, ureas, and thioureas which are antibacterial agents. U.S. Pat. No. 5,880,118 describes substituted oxazine and thiazine oxazolidinone antimicrobials. U.S. Pat. No. 6,968,962 describes phenyloxazolidinones having a C—C bond to 4-8 membered heterocyclic rings. U.S. Pat. No. 5,981,528 describes antibiotic oxazolidinone derivatives. U.S. Pat. No. 5,254,577 describes nitrogen heteroaromatic rings attached to phenyloxazolidinone. U.S. Pat. Nos. 5,547,950 and 5,700,799 also describe the phenyl piperazinyl oxazolidinones.

PCT patent application, Ser. No. PCT/US00/28872 describes mixtures of linezolid and other antibacterial agents. The PCT Publication WO93/23384 describes phenyloxazolidinones containing a substituted diazine moiety and their uses as antimicrobials. WO93/09103 describes substituted aryl and heteroaryl-phenyloxazolidinones useful as antibacterial agents. WO90/02744 describes 5-indolinyl-5β-amidomethyloxazolidinones, 3-(fused ring substituted) phenyl-5β-amidomethyloxazolidinones, which are useful as antibacterial agents. European Patent Publication 352,781 describes phenyl and pyridyl substituted phenyl oxazolidinones. European Patent Application 312,000 describes phenylmethyl and pyridinylmethyl substituted phenyl oxazolidinones.

Other references describing various phenyloxazolidinones include U.S. Pat. Nos. 4,801,600 and 4,921,869; Gregory W. A., et al., J. Med. Chem., 32, 1673-81 (1989); Gregory W. A., et al., J. Med. Chem., 33, 2569-78 (1990); Wang C., et al., Tetrahedron, 45, 1323-26 (1989); Brittelli, et al., J. Med. Chem., 35, 1156 (1992); and Bio-organic and Medicinal Chemistry Letters, 9, pp. 2679-2684, 1999.

### SUMMARY OF THE INVENTION

The present inventors have discovered a class of antibiotic compounds containing an oxazolidinone ring which has useful activity against Gram-positive and Gram-negative pathogens, including MRSA and MRCNS.

Accordingly, the present invention describes an oxazolidinone derivative having antibiotic activity, which is a compound and/or a pharmaceutically acceptable salt and/or a stereoisomer of said compound, wherein said compound has the formula (I)

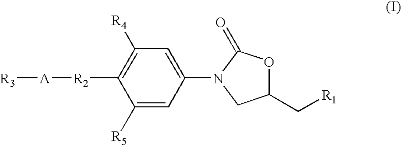

wherein:

R₁ is selected from hydroxy, amino, azido, C₁-C₈ alkyl, C₁-C₈ alkoxy, NHC(═Z)R, wherein Z is O or S, and R is hydrogen or optionally substituted alkyl, alkoxy, cycloalkyl or cycloalkoxy;

R₂ is a five-membered heterocyclic aromatic moiety containing one to three atoms selected from N, O and S;

A is —(CHR^{a})ₙ, wherein R^{a} represents hydrogen or hydroxyl, n represents 1-5;

R₃ is an optionally substituted five or six membered heteroaryl, having at least one nitrogen atom;

n represents 1-5;

R₄ and R₅ are independently selected from hydrogen or fluoro;

with the proviso that when R₁ is NHC(═O)CH₃, and R₂ is imidazole, R₃ is not a substituted triazole;

and including the stereoisomers, prodrugs, and pharmaceutically acceptable salts thereof.

In further aspects of the present invention, the optional substituents on R include halogen, cyano, amino or hydroxyl.

In still further aspects of the present invention, the optional substituents on R₃ include hydrogen, halogen, cyano, hydroxy alkyl, haloalkyl or alkoxy.

The present invention further provides prodrugs of a compound having the formula (I) and methods of preparing prodrugs of a compound having the formula (I).

In accordance with other aspects, the present invention also provides a method of producing antibiotic activity against pathogens in a subject, said method comprising administering to said subject an effective amount of an oxazolidinone derivative of formula (I).

The present invention also provides a method of treating a bacterial infection in a subject, said method comprising administering to said subject an effective amount of an oxazolidinone derivative of formula (I).

The present invention further provides pharmaceutical compositions comprising oxazolidinone derivatives of formula (I) and one or more pharmaceutically-acceptable excipients.

### DETAILED DESCRIPTION OF THE INVENTION

To describe the invention, certain terms are defined herein as follows.

The use of singular includes the use of plural. In a non-limiting example, a recitation of “a derivative” includes a single derivative, as well as multiple derivatives.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices and materials are now described.

The term “compound” is used to denote a molecular moiety of unique, identifiable chemical structure. A molecular moiety (“compound”) may exist in a free species form, in which it is not associated with other molecules. A compound may also exist as part of a larger aggregate, in which it is associated with other molecule(s), but nevertheless retains its chemical identity. A solvate, in which the molecular moiety of defined chemical structure (“compound”) is associated with a molecule(s) of a solvent, is an example of such an associated form. A hydrate is a solvate in which the associated solvent is water. The recitation of a “compound” refers to the molecular moiety itself (of the recited structure), regardless whether it exists in a free form or and an associated forms.

The term “stereoisomers” is used to refer to both optical isomers and geometrical isomers. A recitation of the chemical structure of the compound encompasses all structural variations possible within the structure as shown.

Thus, some of the described compounds have optical centers. If the optical configuration at a given optical center is not defined with specificity, the recitation of chemical structure covers all optical isomers produced by possible configurations at the optical center. The term “optical isomer” defines a compound having a defined optical configuration at least one optical center. This principle applies for each structural genus described herein, as well as for each subgenus and for individual structures. For example, the recitation of a molecular portion as

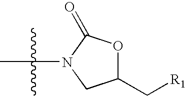

encompasses optical isomers with R and S configurations at the optical center (which arises when R¹ and R² are not identical):

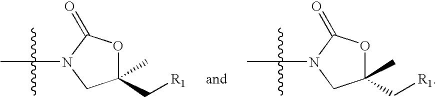

For the purpose of additional illustration, the recitation “a compound of the structure”, for example:

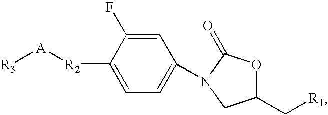

generically encompasses both enantiomers individually, such as:

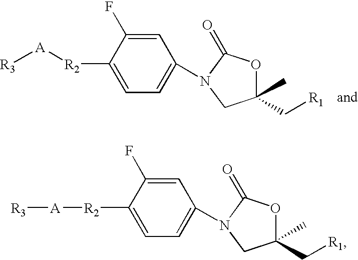

as well as the racemic mixture thereof.

The individual optical isomers may be obtained by using reagents in such a way to obtain single isomeric form in the process wherever applicable or by conducting the reaction in the presence of reagents or catalysts in their single enantiomeric form. Some of the preferred methods of resolution of racemic compounds include use of microbial resolution, resolving the diastereomeric salts, amides or esters formed with chiral acids such as mandelic acid, camphorsulfonic acid, tartaric acid, lactic acid, and the like, wherever applicable or chiral bases such as brucine, cinchona alkaloids and their derivatives, and the like. Commonly used methods are compiled by Jaques et al. in “Enantiomers, Racemates and Resolution” (Wiley Interscience, 1981). Where appropriate the compounds of formula (I) may be resolved by treating with chiral amines, aminoacids, aminoalcohols derived from aminoacids; conventional reaction conditions may be employed to convert acid into an amide; the diastereomers may be separated either by fractional crystallization or chromatography and the stereoisomers of compound of formula (I) may be prepared by hydrolyzing the pure diastereomeric amide, ester or salt.

Some of the described compounds may exist as geometrical isomers (e.g., (E), (Z), etc.). If the geometrical configuration is not self-evident from the structure shown, the recitation of the structure generically covers all possible geometrical isomers. This principle applies for each structural genus described herein, as well as for each subgenus and for individual structures.

The compounds of formula (I) described herein may form salts and thus, can be administered to a subject in the salt form. The term “derivative” is used as a common term for the compound and its salts. Thus, the claim language “a derivative, which is a compound and/or a pharmaceutically-acceptable salt of said compound” is used to define a genus that includes any form of the compound of the given chemical structure and the salts of the recited compound. The use of the term “and/or” is intended to indicate that, for a compound of a given chemical structure, a claim to a “derivative” covers the compound individually, all of its salts individually, and the mixtures of compounds and the salt(s). The term “pharmaceutically-acceptable salts” is intended to denote salts that are suitable for use in human or animal pharmaceutical products. The use of the term “pharmaceutically-acceptable” is not intended to limit the claims to substances (“derivatives”) found only outside of the body.

A “composition” may contain one compound or a mixture of compounds. A “pharmaceutical composition” is any composition useful or potentially useful in producing physiological response in a subject to which such pharmaceutical composition is administered. The term “pharmaceutically acceptable,” with respect to an excipient, is used to define non-toxic substances generally suitable for use in human or animal pharmaceutical products.

As used herein, the term “alkyl,” is intended to include both branched and straight-chain saturated or unsaturated aliphatic hydrocarbon groups having a specified number of carbon atoms. Preferably the alkyl groups of the invention have from 1 to 10 carbon atoms. Branched means that one or more lower alkyl groups such as methyl, ethyl or propyl, are attached to a linear alkyl chain. Non-limiting examples of suitable alkyl groups include methyl, ethyl, n-propyl, isopropyl, n-butyl, and t-butyl. Exemplary ‘alkyl’ groups include methyl, ethyl, propyl, isopropyl and the like.

As used herein, the term “cycloalkyl,” is intended to include non-aromatic mono- or multicyclic ring systems comprising about 3 to about 10 carbon atoms. Exemplary ‘cycloalkyl’ groups, include cyclopropyl, cyclobutyl, cyclopentyl, and the like.

As used herein, the term “alkoxy,” is intended to mean a chain of carbon atoms bonded to an oxygen atom and is defined as ‘alkyl-O—’, wherein the alkyl group is as defined above. The chains of carbon atoms of the alkoxy groups described and claimed herein are saturated, may be straight chain or branched. Exemplary ‘alkoxy’ groups include methoxy, ethoxy, propoxy, isopropoxy and the like.

As used herein, the term “cycloalkoxy” is intended to mean “cycloalkyl-O—”, wherein alkyl group is as defined as above. Exemplary ‘cycloalkoxy’ group includes cyclopropoxy, cyclobutoxy, cyclopentoxy and the like.

As used herein, a “five or six membered heteroaryl” means an aromatic monocyclic ring system comprising about 3 to about 10 ring atoms, preferably about 3 to about 6 atoms, in which one or more of the ring atoms is an element other than carbon, for example O, S or N alone or in combination. The heteroaryl may be optionally substituted by replacing an available hydrogen on the ring by one or more substituents, which may be the same or different. The prefix aza, oxa or thia before the heteroaryl root name means that at least a nitrogen, oxygen or sulfur atom respectively, is present as a ring atom. Exemplary five membered heteroaryl groups include pyrrole, imidazole, triazole ([1,2,3]triazole and [1,2,4]triazole, [1,3,4]triazole), thiazole, oxazole, isooxazole, pyrazole, [1,2,4]oxadiazole & [1,3,4], [1,3,4]thiadiazole, and the like. Exemplary six membered heteroaryl groups include pyridine, pyrimidine, and the like.

The term “substituted”, as used herein, means that one or more hydrogens on the designated atom are replaced with a selection from the indicated groups, provided that the designated atom's normal valency is not exceeded, and that the substitution results in a stable compound.

The terms “individual,” “subject,” and “patient” refer to any subject for whom diagnosis, treatment, or therapy is desired. In one embodiment, the individual, subject, or patient is a human. Other subjects may include animals including, but not limited to cattle, sheep, horses, dogs, cats, guinea pigs, rabbits, rats, primates, opossums and mice.

The terms “treatment,” “treating,” “treat,” and the like are used herein to refer generally to obtaining a desired pharmacological and/or physiological effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a subject, particularly a human, and includes: (a) preventing the disease or symptom from occurring in a subject which may be predisposed to the disease or symptom, but has not yet been diagnosed as having it; (b) inhibiting the disease symptom, i.e., arresting its development; or (c) relieving the disease symptom, i.e., causing regression of the disease or symptom.

The term “prodrug” is used to refer to a compound (and/or its salt) capable of converting, either directly or indirectly, into compounds described herein by the action of enzymes, gastric acid and the like under in vivo physiological conditions (e.g., enzymatic oxidation, reduction and/or hydrolysis). Prodrugs of the present application may be prepared from compound having the formula (I) in a known manner. Conventional procedures for the selection and preparation of suitable prodrug derivatives are described, for example, in DESIGN OF PRODRUGS (1985); Wihnan, 14 BIOCHEM. SOC. TRANS. 375-82 (1986); STELLA ET AL., Prodrugs: A Chemical Approach to Targeted Drug Delivery in DIRECTED DRUG DELIVERY 247-67 (1985), each of which is incorporated by reference herein in its entirety.

The term “therapeutically effective amount” shall mean that amount of a drug or pharmaceutical agent that will elicit the biological or medical response of a tissue, system or patient that is being sought.

One embodiment of the present invention provides an oxazolidinone derivative, which is a compound and/or a pharmaceutically acceptable salt and/or a stereoisomer of said compound, wherein said compound has the formula (I)

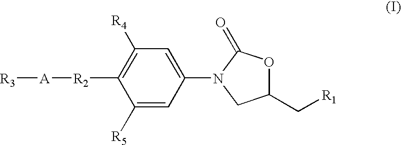

wherein:

R₁ is selected from hydroxy, amino, azido, C₁-C₈ alkyl, C₁-C₈ alkoxy, NHC(═Z)R, wherein Z is O or S, and R is hydrogen or optionally substituted alkyl, alkoxy, cycloalkyl or cycloalkoxy;

R₂ is a five-membered heterocyclic aromatic moiety containing one to three atoms selected from N, O and S;

A is —(CHR^{a})ₙ, wherein R^{a} represents hydrogen or hydroxyl, n represents 1-5;

R₃ is an optionally substituted five or six membered heteroaryl, having at least one nitrogen atom;

n represents 1-5;

R₄ and R₅ are independently selected from hydrogen or fluoro;

with the proviso that when R₁ is NHC(═O)CH₃, and R₂ is imidazole, R₃ is not a substituted triazole;

and including the stereoisomers, prodrugs, and pharmaceutically acceptable salts thereof.

One aspect (Aspect-I) of the present invention provides a compound of formula (I), wherein —R₃-A-R₂— represents:

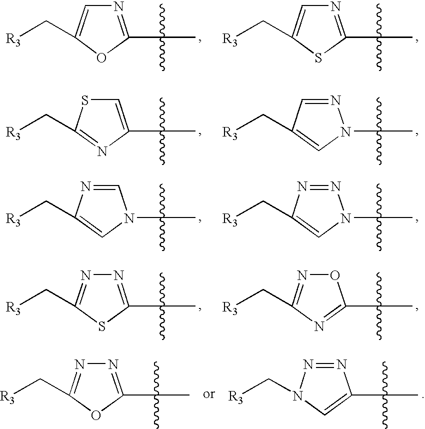

According to the above aspect, the present invention provides compound of formula (I) (Aspect-II), wherein R₃ represents optionally substituted heteroaryl rings selected from

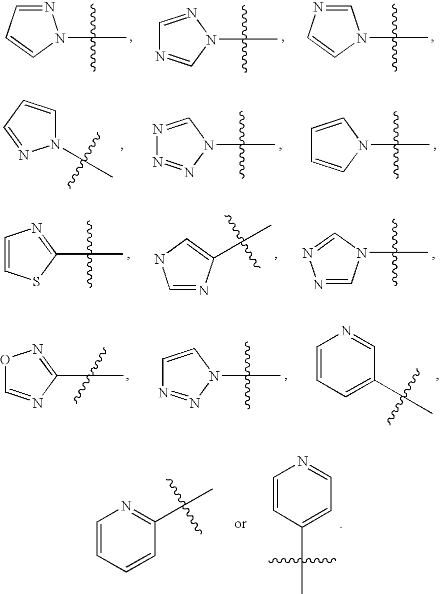

According to the above Aspect-I and Aspect-II, the present invention provides compounds of formula (I), wherein R₁ represents —NHC(═O)R, and wherein R represents optionally substituted alkyl or cycloalkyl.

According to the above Aspect-I and Aspect-II, the present invention provides compounds of formula (I), where R₁ represents —NHC(═O)R, wherein R represents optionally substituted alkoxy or cycloalkoxy.

According to the above Aspect-I and Aspect-II, the present invention provides compounds of formula (I), where R₁ represents —NHC(═S)R, wherein R represents optionally substituted alkyl or cycloalkyl.

According to the above Aspect-I and Aspect-II, the present invention provides compounds of formula (I), where R₁ represents —NHC(═S)R, wherein R represents optionally substituted alkoxy or cycloalkoxy.

Another aspect of the present invention provides a compound of formula (II),

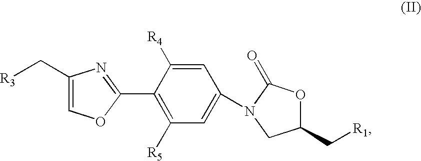

wherein R₁ and R₃ are as defined for formula (I).

Another aspect of the present invention provides a compound of formula (II), wherein R₃ represents optionally substituted:

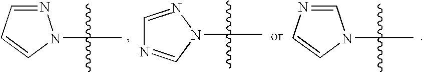

Another aspect of the present invention provides a compound of formula (III),

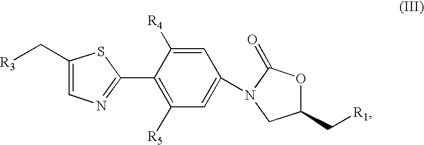

wherein R₁ and R₃ are as defined for formula (I).

Another aspect of the present invention provides a compound of formula (III), wherein R₃ represents optionally substituted:

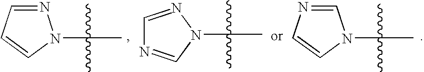

Another aspect of the present invention provides a compound of formula (IV),

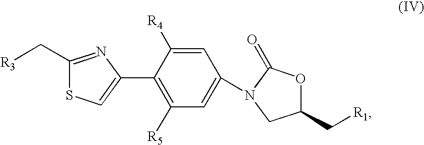

wherein R₁ and R₃ are as defined for formula (I).

Another aspect of the present invention provides a compound of formula (IV), wherein R₃ represents optionally substituted:

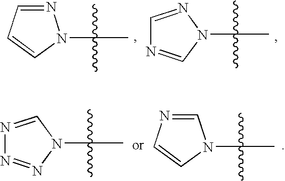

Another aspect of the present invention provides a compound of formula (V),

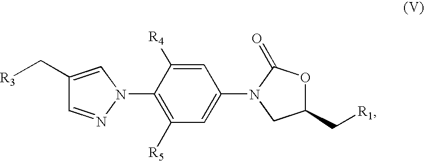

wherein R₁ and R₃ are as defined for formula (I).

Another aspect of the present invention provides a compound of formula (V), wherein R₃ represents optionally substituted:

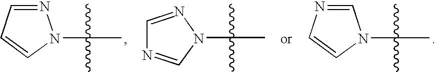

Another aspect of the present invention provides a compound of formula (VI),

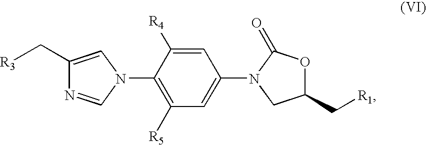

wherein R₁ and R₃ are as defined for formula (I).

Another aspect of the present invention provides a compound of formula (VI), wherein R₃ represents optionally substituted

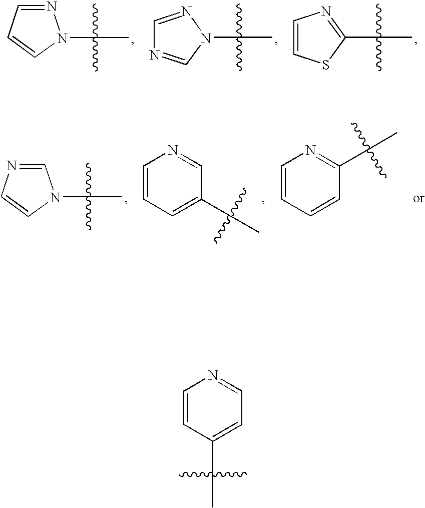

Another aspect of the present invention provides a compound of formula (VII),

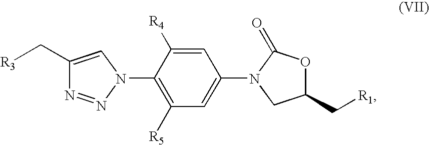

wherein R₁ and R₃ are as defined for formula (I).

Another aspect of the present invention provides a compound of formula (VII), wherein R₃ represents optionally substituted:

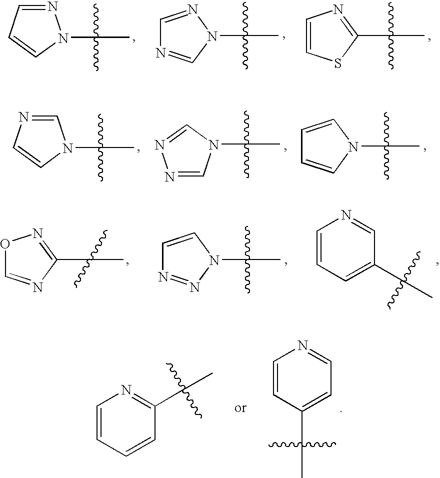

Another aspect of the present invention provides a compound of formula (VIII),

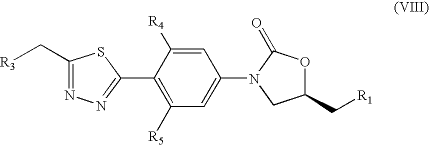

wherein R₁ and R₃ are as defined for formula (I).

Another aspect of the present invention provides a compound of formula (VIII), wherein R₃ represents optionally substituted:

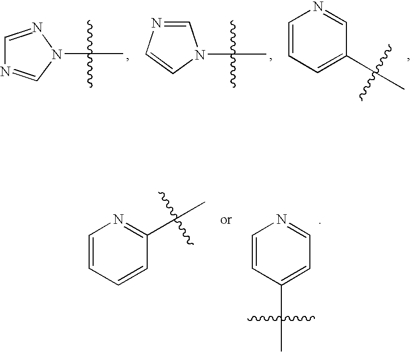

Another aspect of the present invention provides a compound of formula (IX),

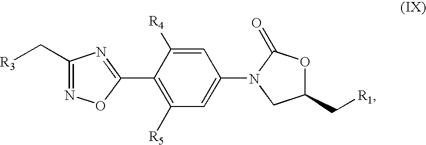

wherein R₁ and R₃ are as defined for formula (I).

Another aspect of the present invention provides a compound of formula (IX), wherein R₃ represents optionally substituted:

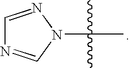

Another aspect of the present invention provides a compound of formula (X),

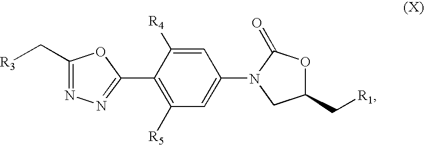

wherein R₁ and R₃ are as defined for formula (I).

Another aspect of the present invention provides a compound of formula (X), wherein R₃ represents optionally substituted

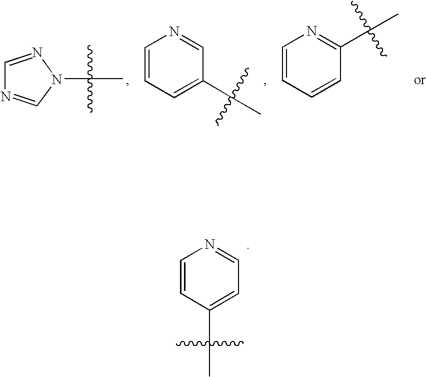

Another aspect of the present invention provides a compound of formula (XI),

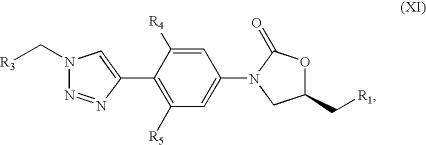

wherein R₁ and R₃ are as defined for formula (I).

Another aspect of the present invention provides a compound of formula (XI), wherein R₃ represents optionally substituted:

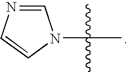

Another aspect of the present invention provides a compound of formula (I), having a basic salt chosen from the salts of Li, Na, K, Ca, Mg, Fe, Cu, Zn, Mn and Al; salts of organic bases; salts of natural amino acids; salts of guanidine; and salts of ammonium.

In another embodiment of the present invention, the compound of formula (I) can be provided along with a “pharmaceutically acceptable carrier” or “pharmaceutically acceptable excipient”, both of which are used interchangeably herein, to form a pharmaceutical composition.

Another aspect of the present invention provides for prodrugs of the compounds of formula (I), including, for example, the following:

STRUCTURES

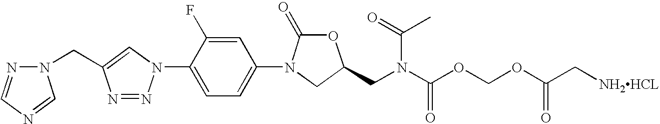

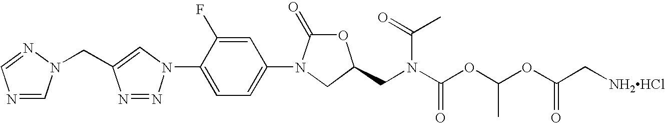

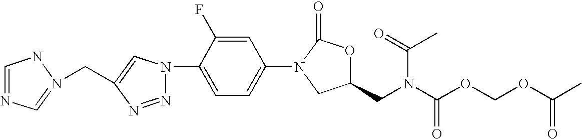

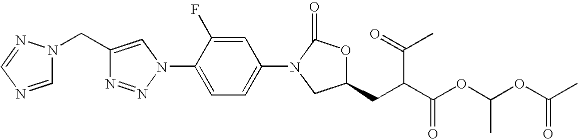

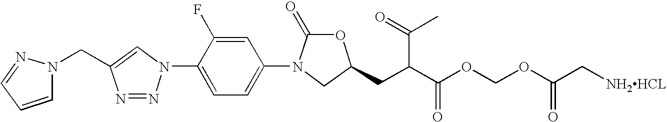

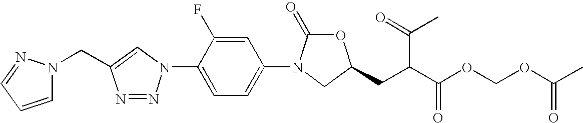

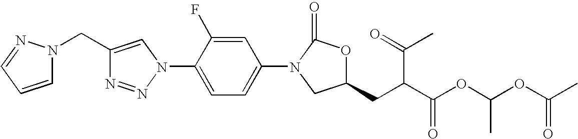

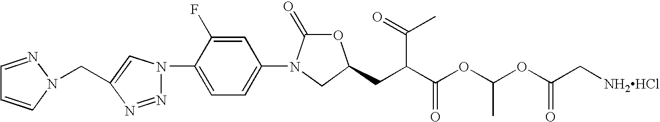

Another aspect of the present invention provides a method of producing antibacterial activity against pathogens in a subject, said method comprising administering to said subject an effective amount of an oxazolodinone derivative of formula (I). In one variant of this aspect, the pathogen is a Gram positive pathogen. In another variant of this aspect, the pathogen is a Gram negative pathogen. In another variant of this aspect, the pathogen is an antibiotic-resistant Gram positive pathogen. In another variant of this aspect, the pathogen is an antibiotic-resistant Gram negative pathogen.

Another aspect of the present invention provides a method of producing antibacterial activity against pathogens in a subject, said method comprising administering to said subject an effective amount of an oxazolidinone derivative of formula (II).

Another aspect of the present invention provides a method of producing antibacterial activity against pathogens in a subject, said method comprising administering to said subject an effective amount of an oxazolidinone derivative of formula (III).

Another aspect of the present invention provides a method of producing antibacterial activity against pathogens in a subject, said method comprising administering to said subject an effective amount of an oxazolidinone derivative of formula (IV).

Another aspect of the present invention provides a method of producing antibacterial activity against pathogens in a subject, said method comprising administering to said subject an effective amount of an oxazolidinone derivative of formula (V).

Another aspect of the present invention provides a method of producing antibacterial activity against pathogens in a subject, said method comprising administering to said subject an effective amount of an oxazolidinone derivative of formula (VI).

Another aspect of the present invention provides a method of producing antibacterial activity against pathogens in a subject, said method comprising administering to said subject an effective amount of an oxazolidinone derivative of formula (VII).

Another aspect of the present invention provides a method of producing antibacterial activity against pathogens in a subject, said method comprising administering to said subject an effective amount of an oxazolidinone derivative of formula (VIII).

Another aspect of the present invention provides a method of producing antibacterial activity against pathogens in a subject, said method comprising administering to said subject an effective amount of an oxazolidinone derivative of formula (IX).

Another aspect of the present invention provides a method of producing antibacterial activity against pathogens in a subject, said method comprising administering to said subject an effective amount of an oxazolidinone derivative of formula (X).

Another aspect of the present invention provides a method of producing antibacterial activity against pathogens in a subject, said method comprising administering to said subject an effective amount of an oxazolidinone derivative of formula (XI).

Another aspect of the present invention provides a pharmaceutical composition comprising an oxazolidinone derivative of formula (I) and one or more pharmaceutically-acceptable excipients.

Another aspect of the present invention provides a pharmaceutical composition comprising an oxazolidinone derivative of formula (II) and one or more pharmaceutically-acceptable excipients.

Another aspect of the present invention provides a pharmaceutical composition comprising an oxazolidinone derivative of formula (III) and one or more pharmaceutically-acceptable excipients.

Another aspect of the present invention provides a pharmaceutical composition comprising an oxazolidinone derivative of formula (IV) and one or more pharmaceutically-acceptable excipients.

Another aspect of the present invention provides a pharmaceutical composition comprising an oxazolidinone derivative of formula (V) and one or more pharmaceutically-acceptable excipients.

Another aspect of the present invention provides a pharmaceutical composition comprising an oxazolidinone derivative of formula (VI) and one or more pharmaceutically-acceptable excipients.

Another aspect of the present invention provides a pharmaceutical composition comprising an oxazolidinone derivative of formula (VII) and one or more pharmaceutically-acceptable excipients.

Another aspect of the present invention provides a pharmaceutical composition comprising an oxazolidinone derivative of formula (VIII) and one or more pharmaceutically-acceptable excipients.

Another aspect of the present invention provides a pharmaceutical composition comprising an oxazolidinone derivative of formula (IX) and one or more pharmaceutically-acceptable excipients.

Another aspect of the present invention provides a pharmaceutical composition comprising an oxazolidinone derivative of formula (X) and one or more pharmaceutically-acceptable excipients.

Another aspect of the present invention provides a pharmaceutical composition comprising an oxazolidinone derivative of formula (XI) and one or more pharmaceutically-acceptable excipients.

Examples of compounds of formula (I) which are described by the present invention, include, but are not limited to the following compounds:

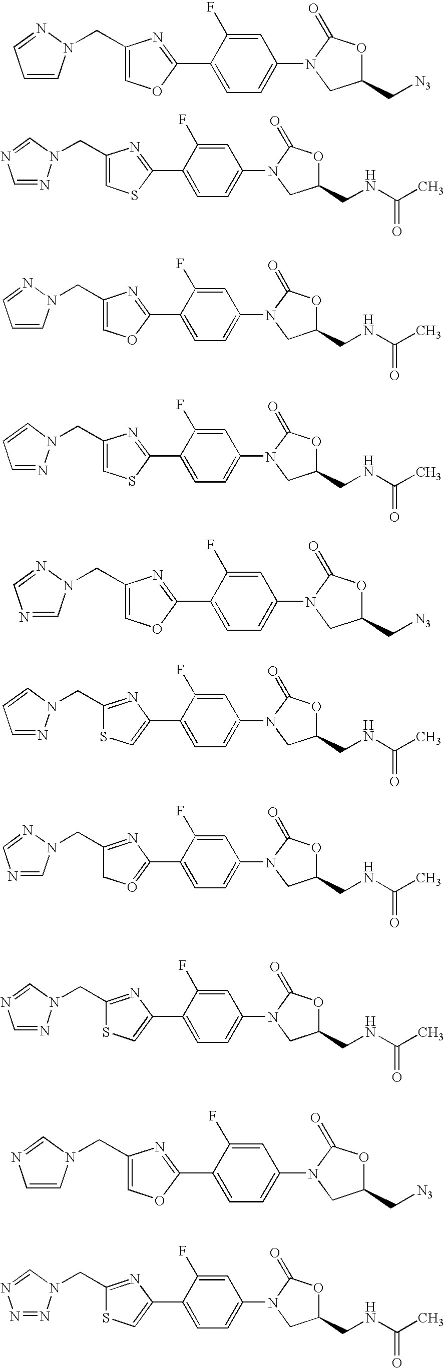

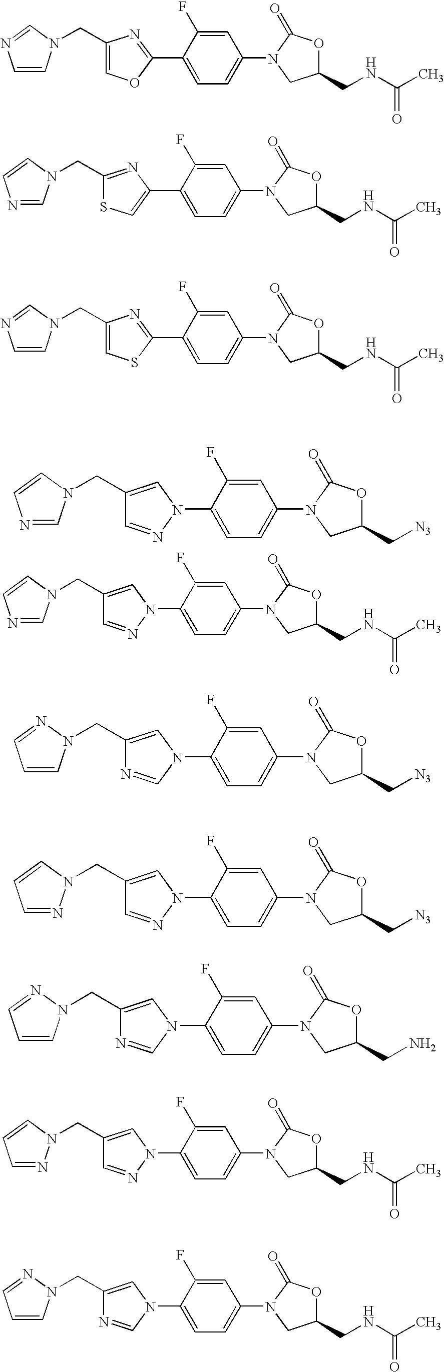

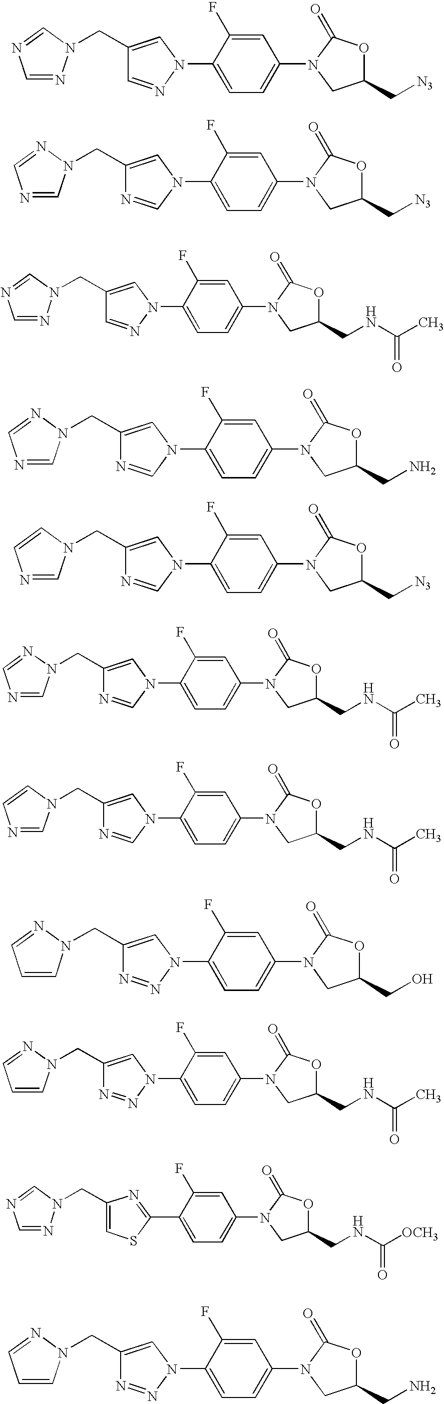

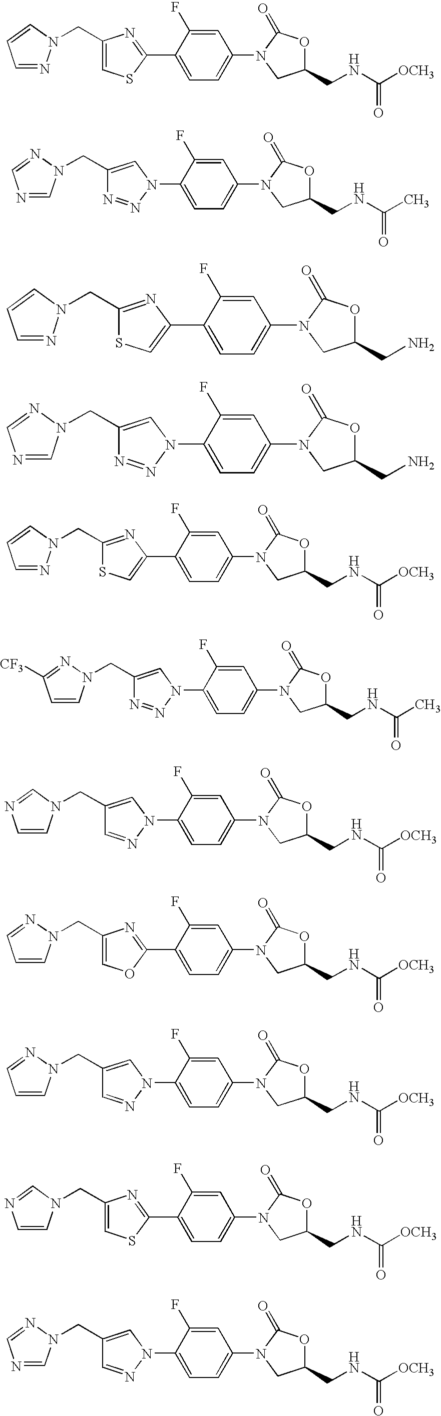

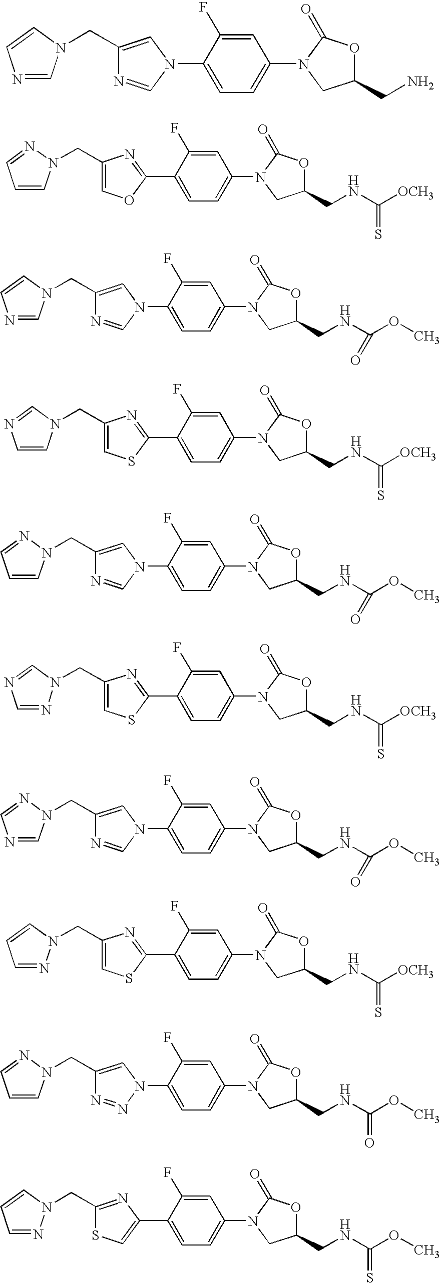

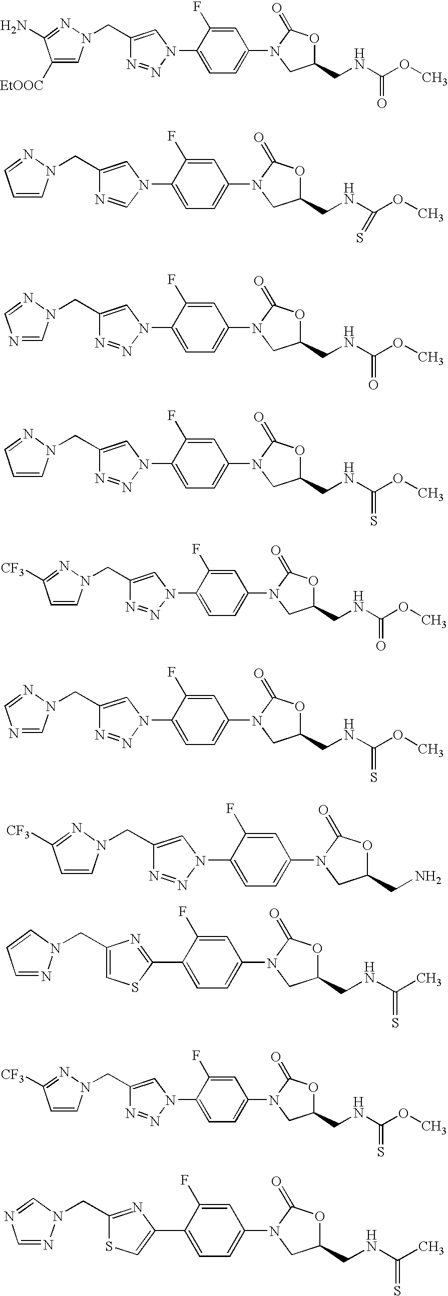

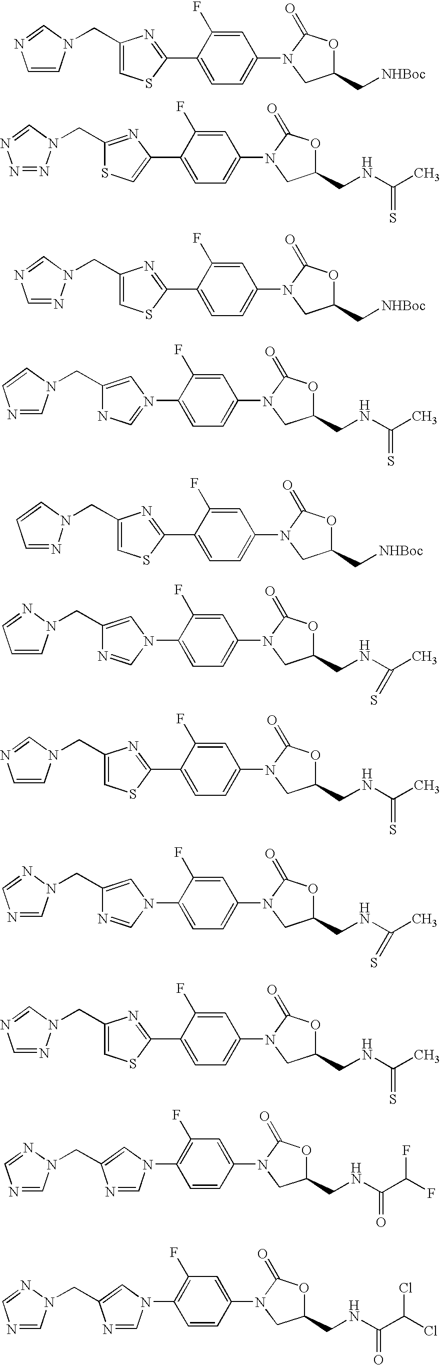

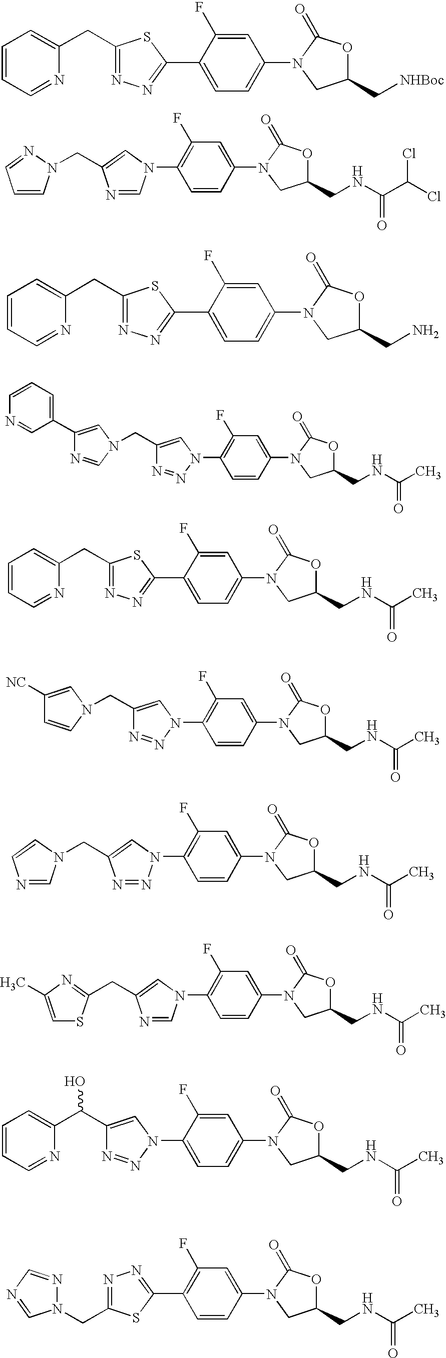

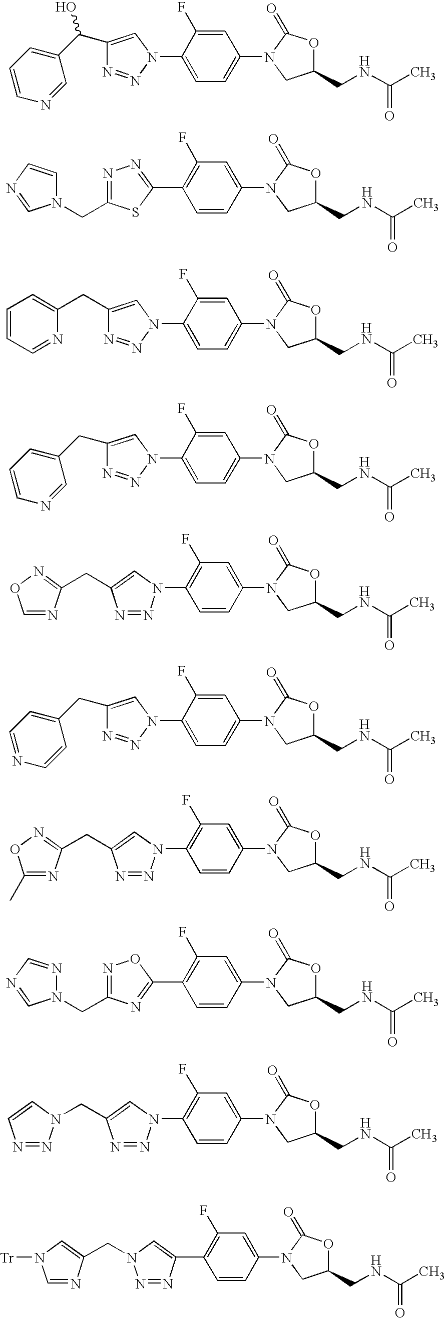

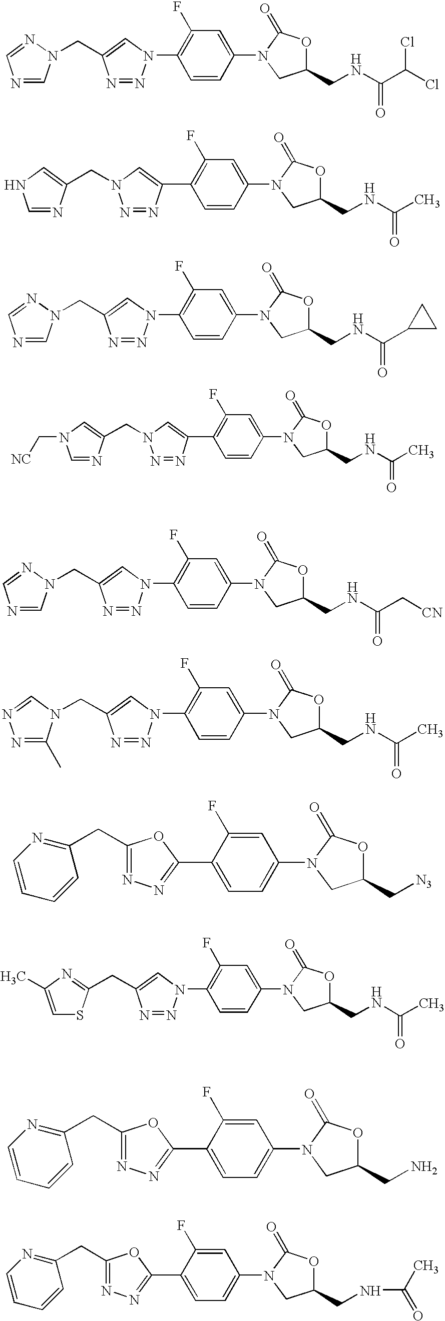

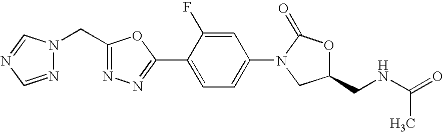

Another embodiment of the present invention provides preparation of the novel compounds of the present invention according to the procedure of the following schemes, using appropriate materials. Those skilled in the art will readily understand that known variations of the conditions and processes of the following preparative procedures can be used to prepare these compounds. All temperatures are in degrees Celsius unless otherwise noted.

In another aspect, the compounds of the present invention have a chiral centre at the C5-position, which have the formula (IA)

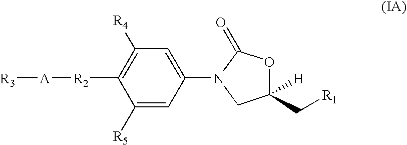

The present invention includes the pure enantiomer or diastereomer depicted above and mixtures of the 5(R) and 5(S) enantiomers or diastereomers, for example a racemic mixture or equal mixtures of diastereomers.

The individual optical isomers or required isomers may be obtained by using reagents in such a way to obtain single isomeric form in the process wherever applicable or by conducting the reaction in the presence of reagents or catalysts in their single enantiomeric form. Some of the preferred methods of resolution of racemic compounds include use of microbial resolution, resolving the diastereomeric salts formed with chiral acids such as mandelic acid, camphorsulfonic acid, tartaric acid, lactic acid, and the like wherever applicable or chiral bases such as brucine, cinchona alkaloids and their derivatives and the like. Commonly used methods are compiled by Jaques et al in “Enantiomers, Racemates and Resolution” (Wiley Interscience, 1981). Where appropriate the compounds of formula (I) may be resolved by treating with chiral amines, aminoacids, aminoalcohols derived from aminoacids; conventional reaction conditions may be employed to convert acid into an amide; the diastereomers may be separated either by fractional crystallization or chromatography and the stereoisomers of compound of formula (I) may be prepared by hydrolyzing the pure diastereomeric amide.

Pharmaceutically acceptable salts forming part of this invention include salts derived from inorganic bases such as Li, Na, K, Ca, Mg, Fe, Cu, Zn, Mn; salts of organic bases such as N,N′-diacetylethylenediamine, betaine, caffeine, 2-diethylaminoethanol, 2-dimethylaminoethanol, N-ethylmorpholine, N-ethylpiperidine, glucamine, glucosamine, hydrabamine, isopropylamine, methylglucamine, morpholine, piperazine, piperidine, procaine, purines, theobromine, triethylamine, trimethylamine, tripropylamine, tromethamine, diethanolamine, meglumine, ethylenediamine, N,N′-diphenylethylenediamine, N,N′-dibenzylethylenediamine, N-benzyl phenylethylamine, choline, choline hydroxide, dicyclohexylamine, metformin, benzylamine, phenylethylamine, dialkylamine, trialkylamine, thiamine, aminopyrimidine, aminopyridine, purine, spermidine, and the like; chiral bases like alkylphenylamine, glycinol, phenyl glycinol and the like, salts of natural amino acids such as glycine, alanine, valine, leucine, isoleucine, norleucine, tyrosine, cystine, cysteine, methionine, proline, hydroxy proline, histidine, ornithine, lysine, arginine, serine, threonine, phenylalanine; unnatural amino acids such as D-isomers or substituted amino acids; guanidine, substituted guanidine wherein the substituents are selected from nitro, amino, alkyl such as methyl, ethyl, propyl and the like; alkenyl such as ethenyl, propenyl, butenyl and the like; alkynyl such as ethynyl, propynyl and the like; ammonium or substituted ammonium salts and aluminum salts. Salts may include acid addition salts where appropriate which are, sulphates, nitrates, phosphates, perchlorates, borates, halides, acetates, tartrates, maleates, citrates, succinates, palmoates, methanesulphonates, benzoates, salicylates, hydroxynaphthoates, benzenesulfonates, ascorbates, glycerophosphates, ketoglutarates and the like. Pharmaceutically acceptable solvates may be hydrates or comprising other solvents of crystallization such as alcohols.

The compounds of the present invention may be formulated and administered in a prodrug form. In general, prodrugs comprise functional derivatives of the compounds of the formula (I) which are capable of being enzymatically activated or converted into the more active parent form. Thus, in the treatment methods of the present invention, the term “administering” encompasses the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in Design of Prodrugs (1985). See also, Wihnan, 14 Biochem. Soc. Trans. 375-82 (1986); Stella et al., Prodrugs: A Chemical Approach to Targeted Drug Delivery in Directed Drug Delivery 247-67 (1985).

The pharmaceutical composition may be in the forms normally employed, such as tablets, capsules, powders, syrups, solutions, suspensions and the like, may contain flavorants, sweeteners etc. in suitable solid or liquid carriers or diluents, or in suitable sterile media to form injectable solutions or suspensions. Such compositions typically contain from 0.1 to 50%, preferably 1 to 20% by weight of active compound, the remainder of the composition being pharmaceutically acceptable carriers, diluents or solvents.

Suitable pharmaceutically acceptable carriers include solid fillers or diluents and sterile aqueous or organic solutions. The active ingredient will be present in such pharmaceutical compositions in the amounts sufficient to provide the desired dosage in the range as described above. Thus, for oral administration, the active ingredient can be combined with a suitable solid or liquid carrier or diluent to form capsules, tablets, powders, syrups, solutions, suspensions and the like. The pharmaceutical compositions, may, if desired, contain additional components such as flavourants, sweeteners, excipients and the like. For parenteral administration, the active ingredient can be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable acid addition salts or salts with base of the compounds. Aqueous solutions with the active ingredient dissolved in polyhydroxylated castor oil may also be used for injectable solutions. The injectable solutions prepared in this manner can then be administered intravenously, intraperitoneally, subcutaneously, or intramuscularly, with intramuscular administration being preferred in humans.

For nasal administration, the preparation may contain the active ingredient of the present invention dissolved or suspended in a liquid carrier, in particular an aqueous carrier, for aerosol application. The carrier may contain additives such as solubilizing agents, such as propylene glycol, surfactants, absorption enhancers such as lecithin (phosphatidylcholine) or cyclodextrin or preservatives such as parabenes.

Tablets, dragees or capsules having talc and/or a carbohydrate carried binder or the like are particularly suitable for any oral application. Preferably, carriers for tablets, dragees or capsules include lactose, corn starch and/or potato starch. A syrup or elixir can be used in cases where a sweetened vehicle can be employed.

The dosage regimen utilizing the compounds of the present invention is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician, veterinarian or clinician can readily determine and prescribe the effective amount of the drug required to prevent, counter or arrest the progress of the condition.

Oral dosages of the present invention, when used for the indicated effects, will range between about 0.01 mg per kg of body weight per day (mg/kg/day) to about 500 mg/kg/day.

In the methods of the present invention, the compounds herein described in detail can form the active ingredient, and are typically administered in admixture with suitable pharmaceutical diluents, excipients or carriers (collectively referred to herein as ‘carrier’ materials) suitably selected with respect to the intended form of administration, that is, oral tablets, capsules, elixirs, syrups and the like, and consistent with conventional pharmaceutical practices.

The following schemes describe procedures for making representative compounds of the present invention. Moreover, by utilizing the procedures described in detail, one of ordinary skill in the art can readily prepare additional compounds of the present invention claimed herein.

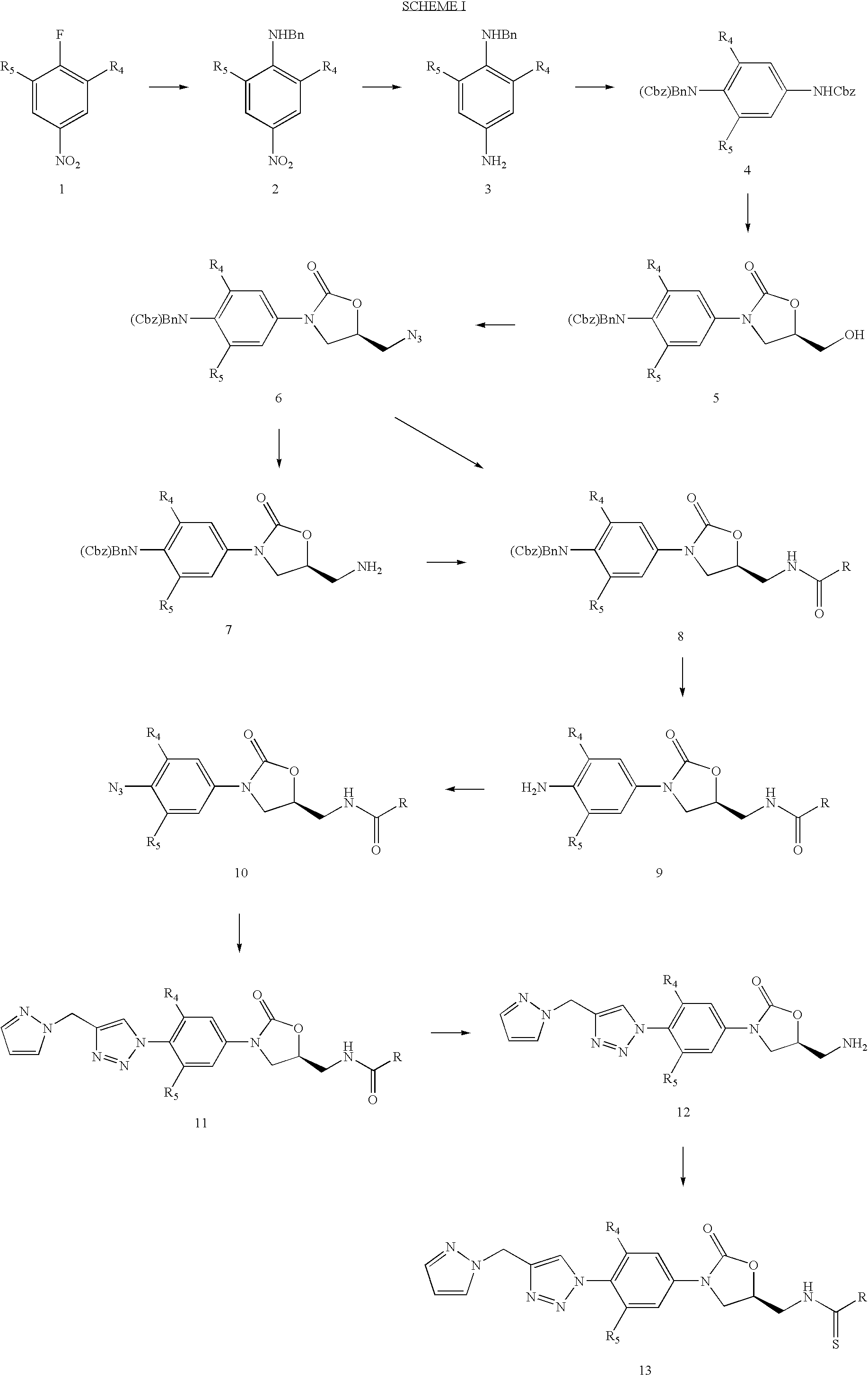

(i) reacting compounds of general formula 1, wherein R₄ and R₅ are same as explained in formula (I), with benzylamine in presence of reagent such as, but not limited to N-ethyl diisopropylamine, in presence of solvent such as, but not limited to dimethylformamide to obtain compounds of general formula 2.
(ii) reacting compounds of general formula 2 with reagent such as, but not limited to nickel chloride hexahydrate, sodium borohydride in presence of solvent such as alcohol (like methanol, ethanol, propanol and the like), to obtain compounds of general formula 3.
(iii) reacting compounds of general formula 3 with benzylchloroformate in presence of reagent such as, but not limited to sodium bicarbonate in presence of solvent such as, but not limited to tetrahydrofuran to get compounds of general formula 4.
(iv) reacting compounds of general formula 4 with R-(−)-glacidyl butrate in presence of reagent such as, but not limited to n-butyl lithium in presence of solvent such as, but not limited to tetrahydrofuran to obtain compounds of general formula 5.
(v) reacting compounds of general formula 5 with methanesulfonyl chloride, sodium azide in presence of reagent such as, but not limited to triethylamine, in presence of solvent such as, but not limited to dichloromethane, dimethylformamide to get compounds of general formula 6.
(vi) reacting compounds of general formula 6 with reagent such as, but not limited to triphenylphosphine, water in presence of solvent such as, but not limited to tetrahydrofuran to obtain compounds of general formula 7.
(vii) compounds of general formula 8 can be prepared by reacting compounds of general formula 7 in presence of reagent such as, but not limited to acetic anhydride, RCOCl in presence of solvent such as, but not limited to pyridine.
(viii) alternatively, reacting compounds of general formula 6 in presence of reagent such as, but not limited to triphenylphosphine and water or palladium/carbon in presence of hydrogen gas, acetic anhydride or RCOCl wherein R is alkyl in presence of solvent such as, but not limited to tetrahydrofuran, pyridine in cooling condition to obtain compounds of general formula 8.
(ix) treating compounds of general formula 8 with any form of palladium/carbon in presence of hydrogen gas and solvent such as, but not limited to methanol, dioxane to obtain compounds of general formula 9.
(x) reacting compounds of general formula 9 with sodium azide in presence of reagent such as, but not limited to hydrogen chloride, sodium nitrite, sodium acetate to get compounds of general formula 10.
(xi) reacting compounds of general formula 10 with 1-prop-2-yne-1H-pyrazol and N-ethyl diisopropylamine in presence of reagent such as, but not limited to cuprous iodide in presence of solvent such as, but not limited to dimethylformamide to obtain compounds of general formula 11.
(xii) reacting compounds of general formula 11 with any acid such as, but not limited to hydrochloric acid in presence of solvent such as alcohol (like methanol, ethanol, propanol and the like), to obtain compounds of general formula 12.
(xiii) reacting compounds of general formula 12 with reagent such as but not limited to thiophosgene, carbon disulfide (CS₂), ethyl chloro formate, RCOCl wherein R is optionally substituted alkoxy, triethylamine, lawessons reagents in presence or absence of solvent such as, but not limited to chloroform, methanol to get compounds of general formula 13 wherein R is optionally substituted alkoxy.

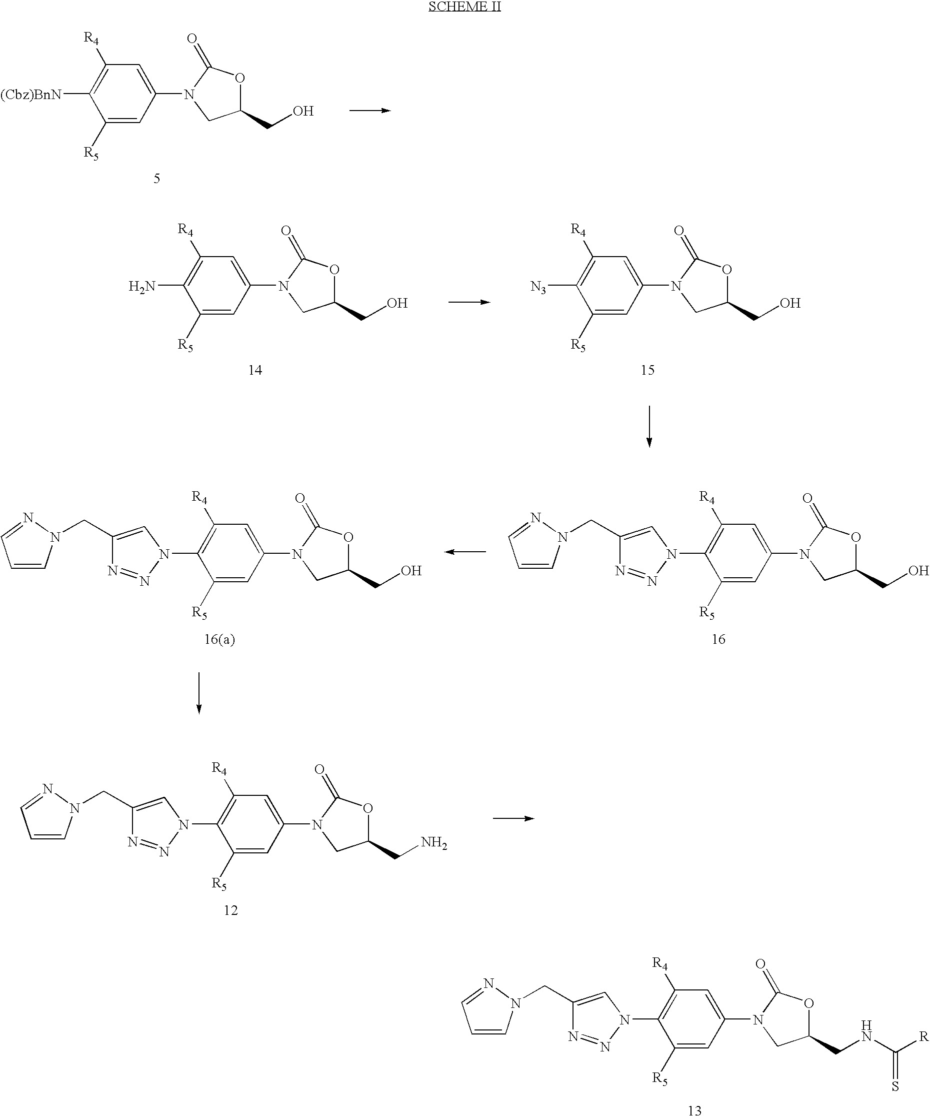

(i) Compound of general formula 5 can be obtained using the methods described in Scheme I.
(ii) treating compounds of general formula 5 with any form of palladium/carbon in presence of hydrogen gas in presence of solvent such as, but not limited to methanol to obtain compounds of general formula 14.
(iii) reacting compounds of general formula 14 with reagent such as but not limited to sodium azide, sodium nitrite, sodium acetate, hydrogen chloride to get compounds of general formula 15.
(iv) reacting compounds of general formula 15 with 1-prop-2-yne-1H-pyrazol and N-ethyl diisopropylamine in presence of reagent such as, but not limited to cuprous iodide in presence of solvent such as, but not limited to dimethylformamide to obtain compounds of general formula 16.
(v) reacting compounds of general formula 16 with methanesulfonyl chloride, sodium azide in presence of reagent such as, but not limited to triethylamine, in presence of solvent such as, but not limited to dichloromethane, dimethylformamide to get compounds of azide compound of general formula 16(a).
(vi) reacting compounds of general formula 16(a) with reagent such as, but not limited to triphenylphosphine, water in presence of solvent such as, but not limited to tetrahydrofuran or palladium/carbon in the presence of hydrogen gas, to obtain compounds of general formula 12.
(vii) reacting compounds of general formula 12 with reagent such as but not limited to thiophosgene, carbon disulfide (CS₂), ethylchloroformate, RCOCl wherein R is optionally substituted alkoxy, triethylamine, lawessons reagents in presence or absence of solvent such as, but not limited to chloroform, methanol to get compounds of general formula 13 wherein R is optionally substituted alkoxy.

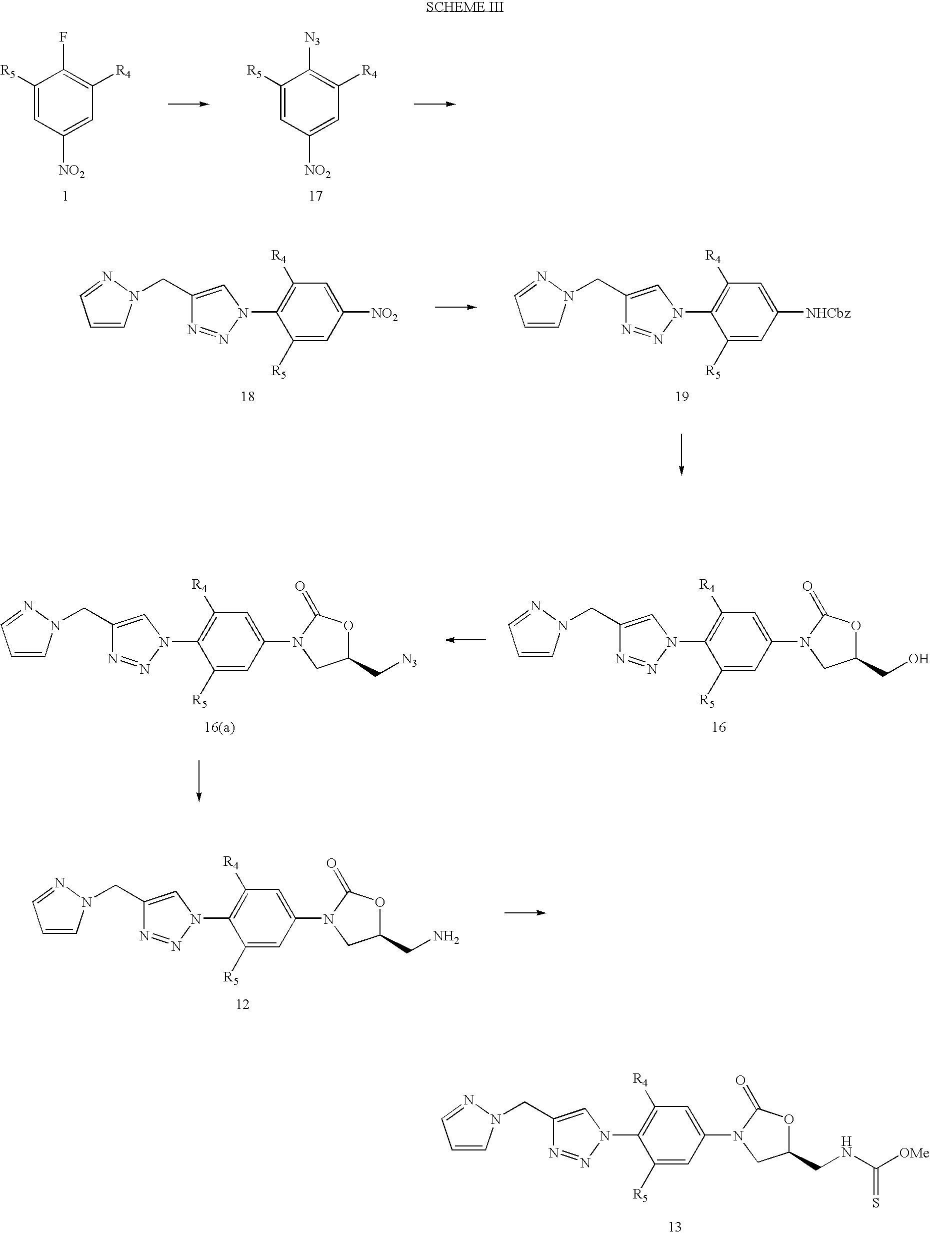

(i) reacting compounds of general formula 1, wherein R₄ and R₅ are same as explained in formula (I), with sodium azide in presence of reagent such as, but not limited to triethylamine, methanesulfonyl chloride in presence of solvent such as, but not limited to dichloromethane, acetonitrile, dimethyl formamide to get compounds of general formula 17.
(ii) reacting compounds of general formula 17 with 1-prop-2-yne-1H-pyrazol and N-ethyl diisopropylamine in presence of reagent such as, but not limited to cuprous iodide in presence of solvent such as, but not limited to dimethylformamide to obtain compounds of general formula 18.
(iii) reacting compounds of general formula 18 with benzylchloroformate in presence of reagent such as, but not limited to sodium bicarbonate in presence of solvent such as, but not limited to tetrahydrofuran to get compounds of general formula 19.
(iv) reacting compounds of general formula 19 with R-(−) glycidyl butyrate in presence of reagent such as, but not limited to n-butyl lithium to obtain compounds of general formula 16.
(v) reacting compounds of general formula 16 with methanesulfonyl chloride, sodium azide in presence of reagent such as, but not limited to triethylamine, in presence of solvent such as, but not limited to dichloromethane, dimethylformamide to get compounds of azide compound of general formula 16(a).
(vi) reacting compounds of general formula 16(a) with reagent such as, but not limited to triphenylphosphine, water in presence of solvent such as, but not limited to tetrahydrofuran to obtain compounds of general formula 12.
(vii) reacting compounds of general formula 12 with reagent such as but not limited to thiophosgene, carbon disulfide (CS₂), ethyl chloro formate, RCOCl wherein R is optionally substituted alkoxy, triethylamine, lawessons reagents in presence or absence of solvent such as, but not limited to chloroform, methanol to get compounds of general formula 13 wherein R is optionally substituted alkoxy.

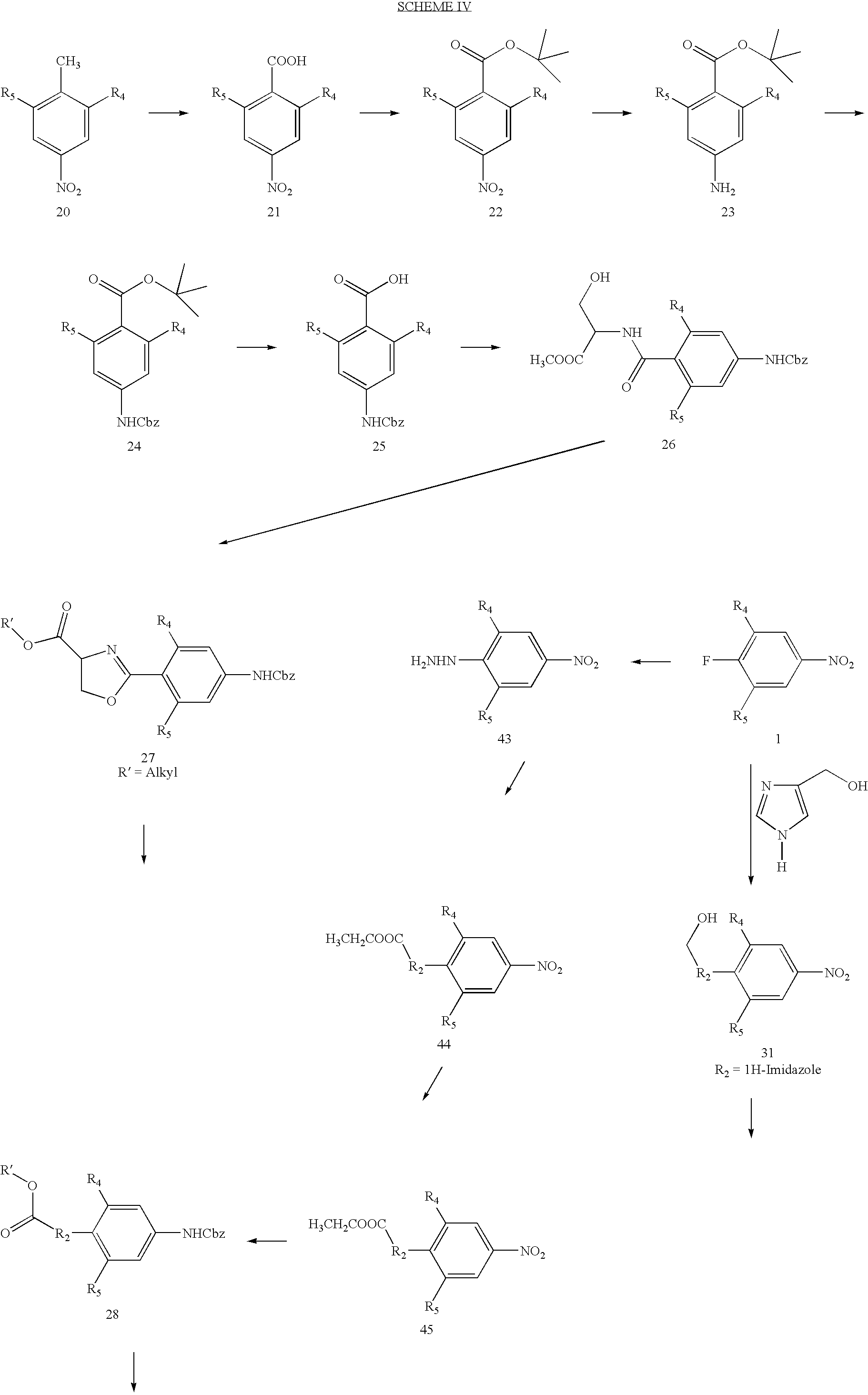

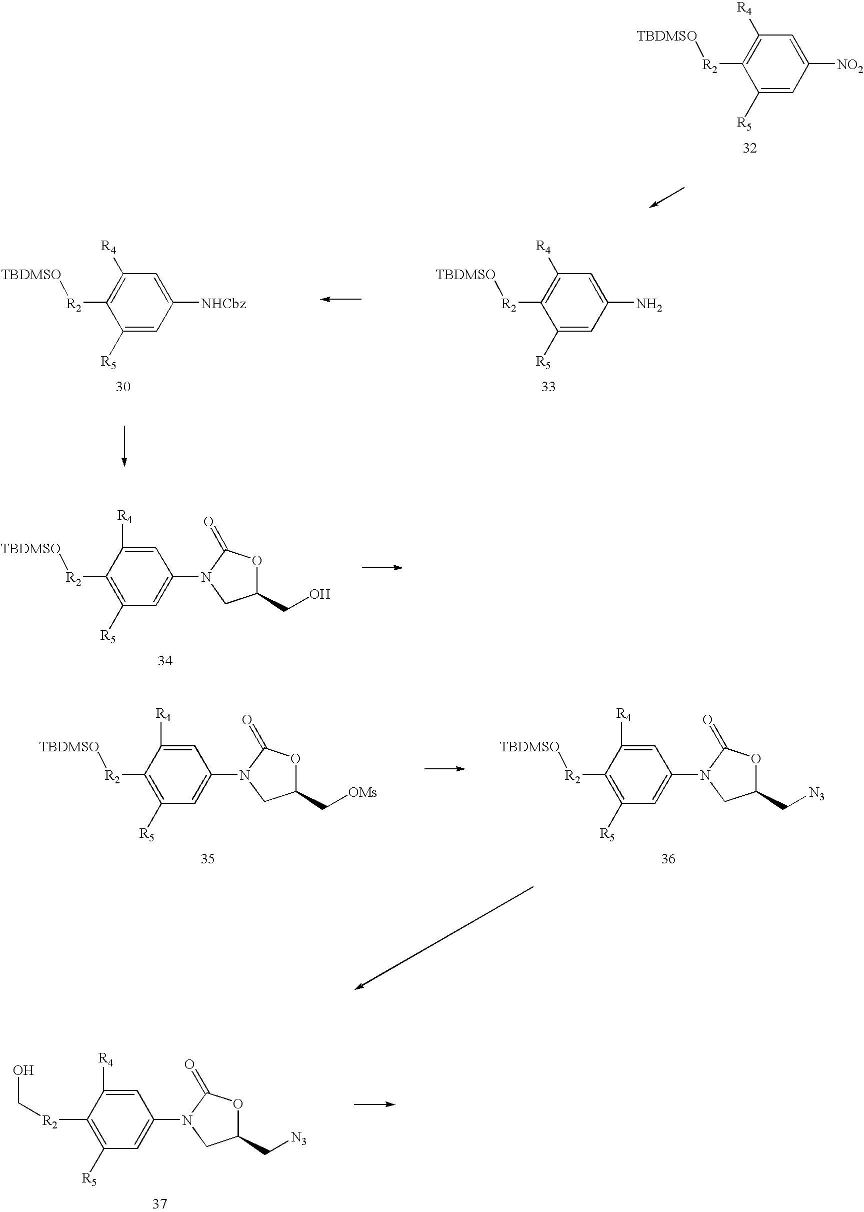

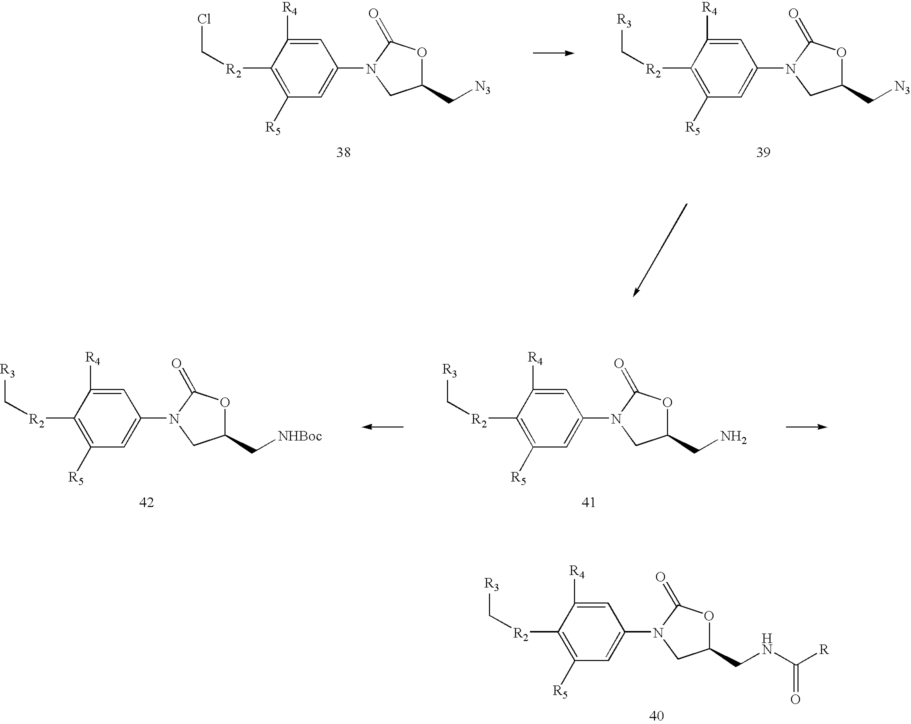

(i) oxidation of compounds of general formula 20 with chromium trioxide or potassium permanganate in presence of reagent such as, but not limited to sulfuric acid, acetic acid to get compounds of general formula 21.
(ii) reacting compounds of general formula 21 in presence of reagent such as, but not limited to tertiary butoxy carbony (BOC) anhydride, 4-(N,N-dimethylamino)pyridine (DMAP), triethyl amine in presence of solvent such as, but not limited to tetrahydrofuran to obtain compounds of general formula 22.
   alternatively compounds of general formula 21 in presence of reagent such as, but not limited to thionyl chloride, lithium tert-butoxide in presence of solvent such as, but not limited to tetrahydrofuran to obtain compounds of general formula 22.
(iii) reduction of compounds of general formula 22 in presence of reagent such as, but not limited to Fe-ammonium chloride in presence of solvent such as, but not limited to ethanol, water to obtain compounds of general formula 23. Alternatively other reducing agents are selected from palladium/carbon in presence of hydrogen gas, Raney nickel and H₂, Sn and HCl.
(iv) reacting compounds of general formula 23 with benzylchloroformate in presence of reagent such as, but not limited to sodium bicarbonate in presence of solvent such as, but not limited to tetrahydrofuran to get compounds of general formula 24.
(v) reacting compounds of general formula 24 with reagent such as, but not limited to trifluoroacetic acid (TFA), hydrochloric acid in presence of solvent such as, but not limited to dichloromethane to obtain compounds of general formula 25.
(vi) reacting compounds of general formula 25 with set of reagent such as, but not limited to N-ethyl-N′-(3-dimethylaminopropyl)carbodiimide (EDC), hydroxybenzotriazole (HOBT), N,N-diisopropylethylamine (DIPEA) or serine methyl ester hydrochloride salt, N,N-dicyclohexylcarbodiimide (DCC) to obtain compounds of general formula 26.
(vii) reacting compounds of general formula 26 with reagent such as, but not limited to burgess reagent in presence of solvent such as, but not limited to tetrahydrofuran in heating condition to obtain compounds of general formula 27 wherein R′ is alkyl.
(viii) (a) reacting compounds of general formula 27 with reagent such as, but not limited to 2,3-dichloro-5,6-dicyano-1,4-benzoquinone (DDQ) in presence of solvent such as, but not limited to 1,4-dioxane to obtain compounds of general formula 28 wherein R₂ is Oxazole.
   (b) alternatively compounds of general formula 28 can be prepared from compounds of general formula 1.
   (i) reacting compounds of general formula 1 with reagent such as, but not limited to hydrazine hydrate in presence of solvent such as, but not limited to ethanol in heating condition to obtain compounds of general formula 43.
   (ii) reacting compounds of general formula 43 with reagent such as, but not limited to 2-formyl-3-oxo-propionic acid ethyl ester, sodium acetate in presence of solvent such as, but not limited to ethanol in heating condition to obtain compounds of general formula 44 wherein R₂ is pyrazole.
   (iii) reacting compounds of general formula 44 with reagent such as, but not limited Fe, ammonium chloride in presence of solvent such as, but not limited to ethanol, water in heating condition to obtain compounds of general formula 45.
   (iv) reacting compounds of general formula 45 with reagent such as, but not limited to benzyl Chloroformate (Cbz-Cl), sodiumbicarbonate in presence of solvent such as, but not limited to tetrahydrofuran to obtain compounds of general formula 28 wherein R₂ is Pyrazole.
(ix) reacting compounds of general formula 28, wherein R₂ is Oxazole or Pyrazole, with reagent such as, but not limited to lithium aluminium hydride (LiAlH₄) in presence of solvent such as, but not limited to tetrahydrofuran, ether, dioxane in cooling condition to obtain compounds of general formula 29.
(x) (a) reacting compounds of general formula 29 with reagent such as, but not limited to tert-butyldimethylsilyl chloride (TBDMS-chloride), imidazole in presence of solvent such as, but not limited to dimethylformamide to obtain compounds of general formula 30.
   (b) alternatively compounds of general formula 30 can be prepared from compounds of general formula 1.
   (i) reacting compounds of general formula 1 with (1H-Imidazol-4-yl)-methanol in presence of reagent such as, but not limited to diisopropylethylamine to obtain compounds of general formula 31 wherein R₂ is imidazole.
   (ii) reacting compounds of general formula 31 with reagent such as, but not limited to TBDMS-chloride, imidazole in presence of solvent such as, but not limited to dimethylformamide to obtain compounds of general formula 32.
   (iii) reacting compounds of general formula 32 with reagent such as, but not limited Fe, ammonium chloride in presence of solvent such as, but not limited to ethanol, water in heating condition to obtain compounds of general formula 33.
   (iv) reacting compounds of general formula 33 with reagent such as, but not limited to benzyl chloroformate (Cbz-Cl), sodiumbicarbonate in presence of solvent such as, but not limited to tetrahydrofuran to obtain compounds of general formula 30 wherein R₂ is imidazole.
(xi) reacting compounds of general formula 30, wherein R₂ is oxazole, pyrazole, 1H-imidazole, with reagent such as, but not limited to n-butyl lithium, R-(−)glycidyl butyrate in presence of solvent such as, but not limited to tetrahydrofuran in cooling condition to obtain compounds of general formula 34.
(xii) reacting compounds of general formula 34 with reagent such as, but not limited to mesyl chloride, triethylamine, triflic anhydride (Tf₂O) in presence of solvent such as, but not limited to dichloromethane in cooling condition to obtain compounds of general formula 35.
(xiii) reacting compounds of general formula 35 with reagent such as, but not limited to sodium azide in presence of solvent such as, but not limited to dimethylformamide in heating condition to obtain compounds of general formula 36.
(xiv) reacting compounds of general formula 36 with reagent such as, but not limited to acetic acid or tetrabutylammonium fluoride (TBAF) in presence of solvent such as, but not limited to tetrahydrofuran, water to obtain compounds of general formula 37.
(xv) chlorination of compounds of general formula 37 with reagent such as, but not limited to thionyl chloride, in presence of solvent such as, but not limited to dichloromethane to obtain compounds of general formula 38.
(xvi) reacting compounds of general formula 38 with any heteroaryl compounds such as, but not limited to triazole, pyrazole, imidazole in presence of reagent such as, but not limited to potassium carbonate in presence of solvent such as, but not limited to acetonitrile in heating condition to obtain compounds of general formula 39 wherein R₃ is triazole, pyrazole, imidazole.
(xvii) (a) reacting compounds of general formula 39 in presence of reagent such as, but not limited to triphenylphosphine and water or palladium/carbon in presence oh hydrogen gas, acetic anhydride or RCOCl wherein R is alkyl in presence of solvent such as, but not limited to tetrahydrofuran, pyridine in cooling condition to obtain compounds of general formula 40.
   (b) alternatively compound of general formula 40 can be prepared by reacting compounds of general formula 41 in presence of reagent such as, but not limited to acetic anhydride, RCOCl in presence of solvent such as, but not limited to pyridine.
   (c) reacting compounds of general formula 39 with reagent such as, but not limited to triphenylphosphine, water in presence of solvent such as, but not limited to tetrahydrofuran to obtain compounds of general formula 41.
(xviii) reacting compounds of general formula 41 in presence of reagent such as, but not limited to di-tert-butyl dicarbonate to obtain compounds of general formula 42.

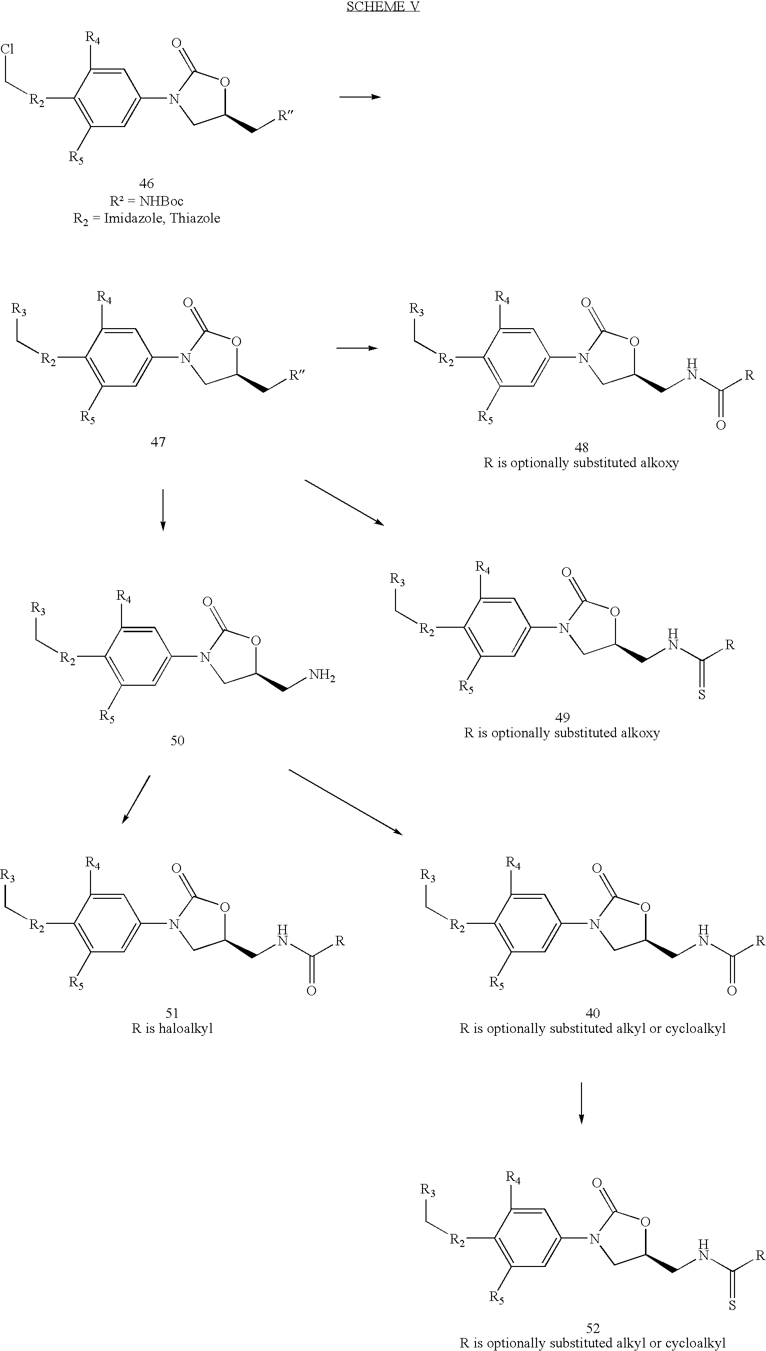

(i) reacting compounds of general formula 46, wherein R₄ and R₅ are same as explained in formula (I), R₂ is oxazole, pyrazole, H-imidazole, thiazole and R″ is —NHBoc, with any heteroaryl compounds such as, but not limited to triazole, pyrazole, imidazole in presence of reagent such as, but not limited to potassium carbonate in presence of solvent such as, but not limited to acetonitrile in heating condition to obtain compounds of general formula 47 wherein R₃ is triazole, pyrazole, imidazole.
(ii) reacting compounds of general formula 47 with reagent such as, but not limited to acid such as trifluoroacetic acid or hydrochloric acid, RCOCl wherein R is optionally substituted alkoxy, in presence of solvent such as, but not limited to dichloromethane to obtain compounds of general formula 48 wherein R is optionally substituted alkoxy.
(iii) reacting compounds of general formula 47 with reagent such as, but not limited to acid such as thiophosgene, trifluoroacetic acid or hydrochloric acid, CS₂, ethyl chloro formate, in presence of solvent such as, but not limited to methanol to obtain compounds of general formula 49, wherein R is optionally substituted alkoxy.
(iv) reacting compounds of general formula 47 with reagent such as, but not limited to acid such as trifluoroacetic acid or hydrochloric acid to obtain compounds of general formula 50.
(v) reacting compounds of general formula 50 with X₂CHCOOH wherein X is halogen in presence of reagent such as, but not limited to N-ethyl-N′-(3-dimethylaminopropyl)carbodiimide (EDCI) to obtain compounds of general formula 51, wherein R is haloalkyl.
(vi) reacting compounds of general formula 50 in presence of reagent such as, but not limited to acetic anhydride, triethyl amine, RCOCl wherein R is optionally substituted alkyl or cycloalkyl, in presence of solvent such as, but not limited to pyridine to obtain compounds of general formula 40.
(vii) reacting compounds of general formula 40 with reagent such as but not limited to Lawessons reagents in presence or absence of solvent such as, but not limited to chloroform, methanol to get compounds of general formula 52 wherein R is optionally substituted alkyl or cycloalkyl.

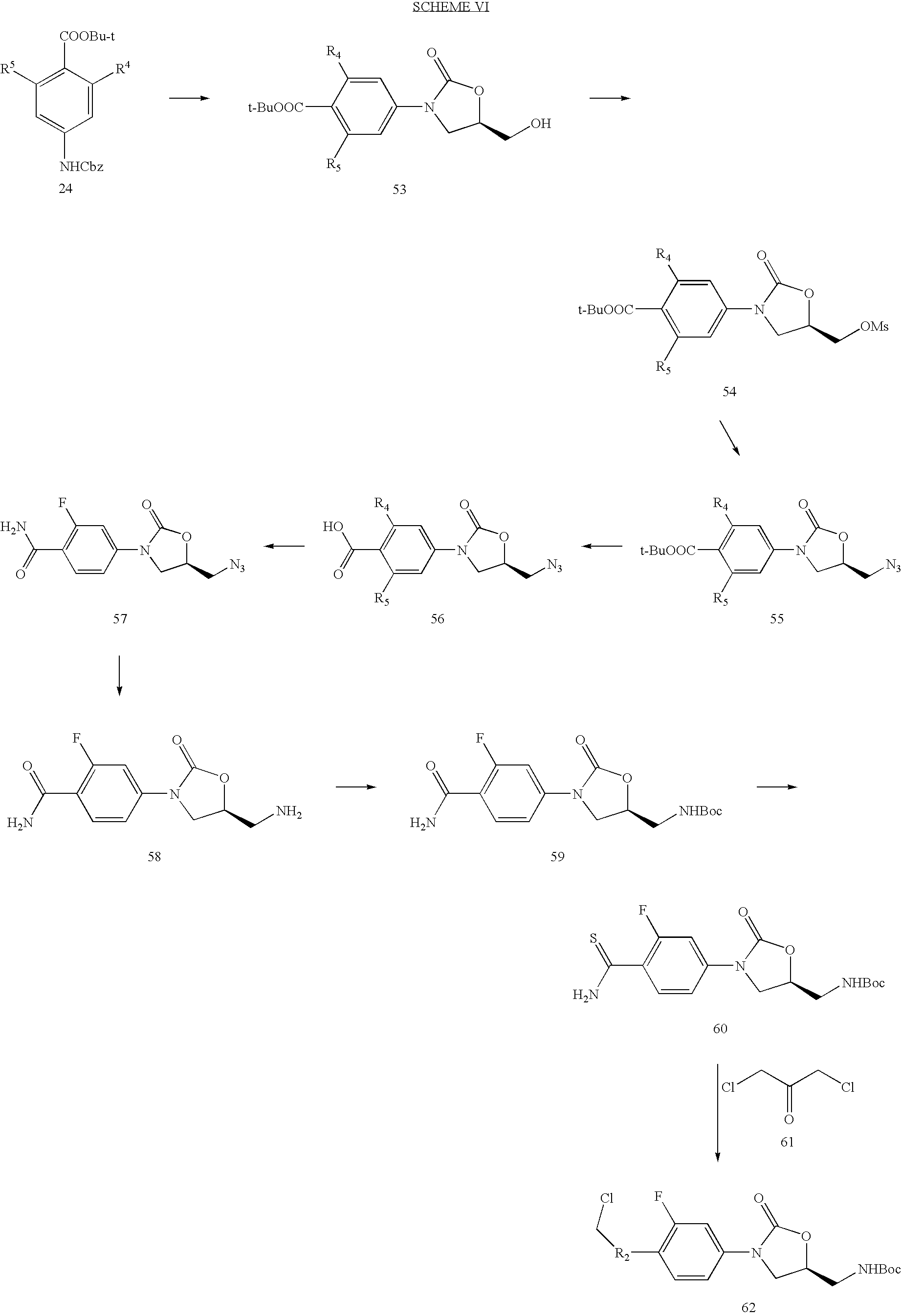

Compound of formula 24 can be obtained by following Scheme IV.
(i) reacting compounds of general formula 24, wherein R₄ and R₅ are same as explained in formula (I), with reagent such as, but not limited to butyl lithium, R-(−)glycidyl butyrate in presence of solvent such as, but not limited to tetrahydrofuran in cooling condition to obtain compounds of general formula 53, wherein R₄ and R₅ are same as explained in formula (I).
(ii) reacting compounds of general formula 53 with reagent such as, but not limited to mesyl chloride, triethylamine, triflic anhydride (Tf₂O) in presence of solvent such as, but not limited to dichloromethane in cooling condition to obtain compounds of general formula 54.
(iii) reacting compounds of general formula 54 with reagent such as, but not limited to sodium azide in presence of solvent such as, but not limited to dimethylformamide in heating condition to obtain compounds of general formula 55.
(iv) reacting compounds of general formula 55 with reagent such as, but not limited to trifluoroacetic acid to obtain compounds of general formula 56.
(v) reacting compounds of general formula 56 with reagent such as, but not limited to thionyl chloride, ammonium hydroxide to obtain compounds of general formula 57.
(vi) reacting compounds of general formula 57 in presence of reagent such as, but not limited to triphenylphosphane, water or palladium/carbon in presence of hydrogen gas to obtain compounds of general formula 58.
(vii) reacting compounds of general formula 58 in presence of reagent such as, but not limited to di-tert-butyl dicarbonate, triethyl amine, potassium hydroxide to obtain compounds of general formula 59.
(viii) reacting compounds of general formula 59 in presence of reagent such as, but not limited to lawesson's reagent to obtain compounds of general formula 60.
(ix) reacting compounds of general formula 60 in presence of reagent such as, but not limited to 1,3-dichloro-propan-2-one to obtain compounds of general formula 62 wherein R₂ is thiazole.

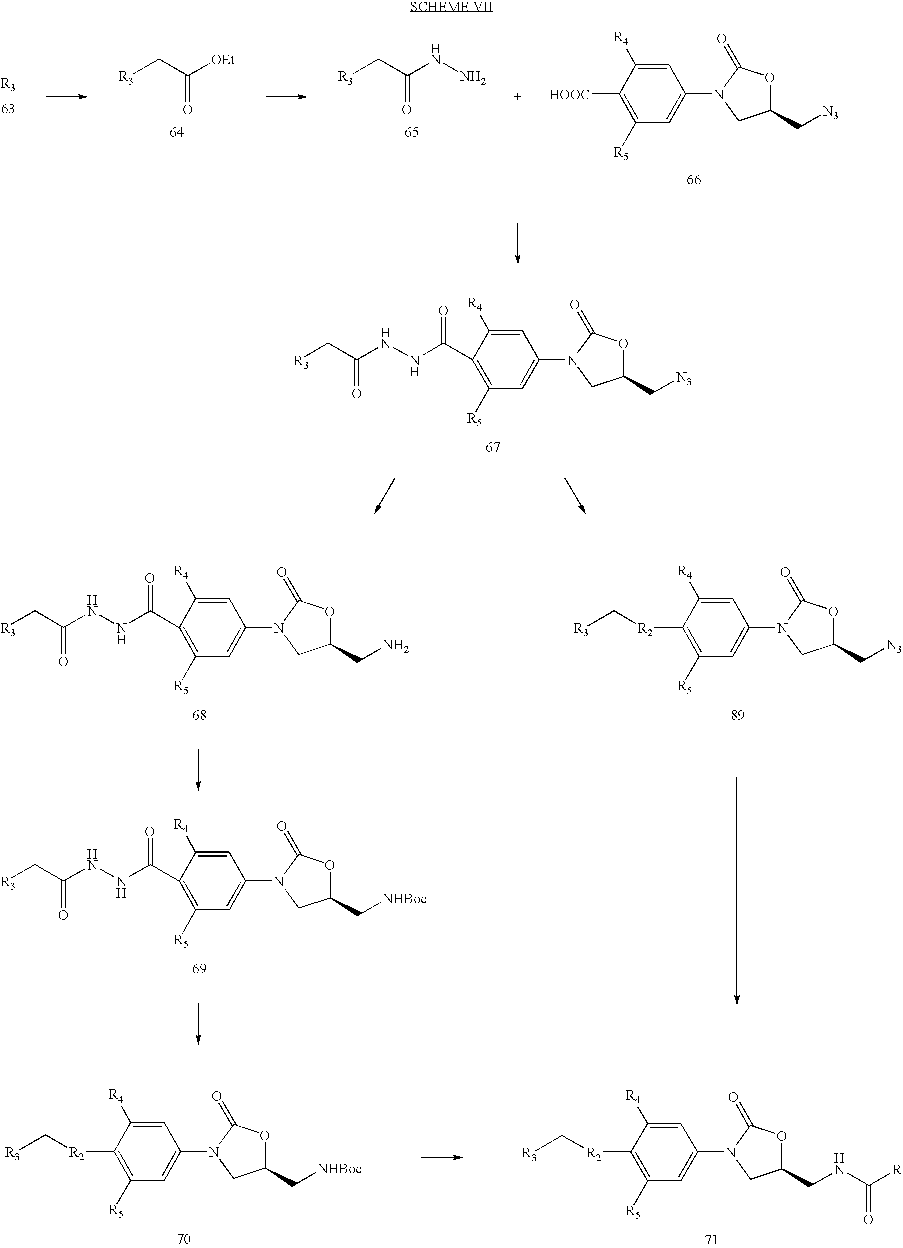

(i) reacting compounds of general formula 63, wherein R₃ is selected from heteroaryl such as triazole or pyridine, with reagent such as, but not limited to potassium carbonate, bromoethylacetate to obtain compounds of general formula 64.
(ii) reacting compounds of general formula 64 with reagent such as, but not limited to hydrazine hydrate to obtain compounds of general formula 65.
(iii) reacting compounds of general formula 65 with compounds of general formula 66, wherein R₄ and R₅ are same as explained in formula (I), in presence of reagent such as, but not limited to thionyl chloride, oxalykyl chloride, N,N′-diisopropylethylamine to obtain compounds of general formula 67.
   alternatively compounds of general formula 67 can be obtained by reacting compound of general formula 65 with compounds of general formula 66, wherein R₄ and R₅ are same as explained in formula (I), in presence of reagent such as, but not limited to N-ethyl-N¢-(3-dimethylaminopropyl)carbodiimide (EDCI), 2-(1H-7-azabenzotriazol-1-yl)-1,1,3,3-tetramethyl uronium hexafluorophosphate (HATU), N,N′-dicyclohexylcarbodiimide (DCC).
(iv) reacting compounds of general formula 67 with reagent such as, but not limited to palladium/carbon in presence of hydrogen gas to obtain compounds of general formula 68.
(v) reacting compounds of general formula 68 in presence of reagent such as, but not limited to di-tert-butyl dicarbonate, triethylamine, potassium hydroxide to obtain compounds of general formula 69.
(vi) reacting compounds of general formula 69 in presence of reagent such as, but not limited to lawesson's reagent to obtain compounds of general formula 70, wherein R₂ is thiadiazole.
(vii) reacting compounds of general formula 70 in presence of reagent such as, but not limited to trifluoroacetic acid, acetic anhydride or R—COCl to obtain compounds of general formula 71, wherein R is optionally substituted alkyl or cycloalkyl.
(viii) Alternatively compound of 71 can be prepared by reacting compound of general formula 64, wherein R₂ is oxadiazole, with reagent such as, but not limited to phosphorus oxychlorid, 4-(N,N-dimethylamino)pyridine in presence of solvent, but not limited to acetonitrile to obtain compounds of general formula 89, wherein R₂ is oxadiazole, which further react with reagent such as palladium/carbon in presence of hydrogen gas and hydrogen or acetic anhydride and triethylamine in presence of methanol or dichloromethane as solvent.

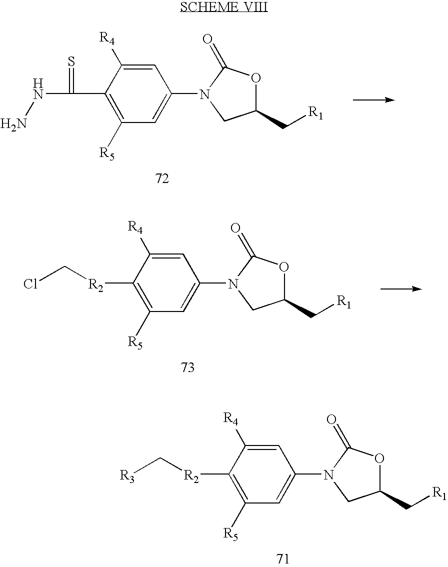

(i) reacting compounds of general formula 72, wherein R₁, R₄ and R₅ are same as explained in formula (I), with chloro acetyl chloride in presence of solvent such as, but not limited to tetrahydrofuran to obtain compounds of general formula 73, wherein R₂ is thiadiazole.
(ii) reacting compounds of general formula 73 with any heteroaryl compounds such as, but not limited to triazole, pyrazole, imidazole in presence of reagent such as, but not limited to potassium carbonate in presence of solvent such as, but not limited to dimethylformamide in heating condition to obtain compounds of general formula 71 wherein R₃ is triazole, pyrazole, imidazole.

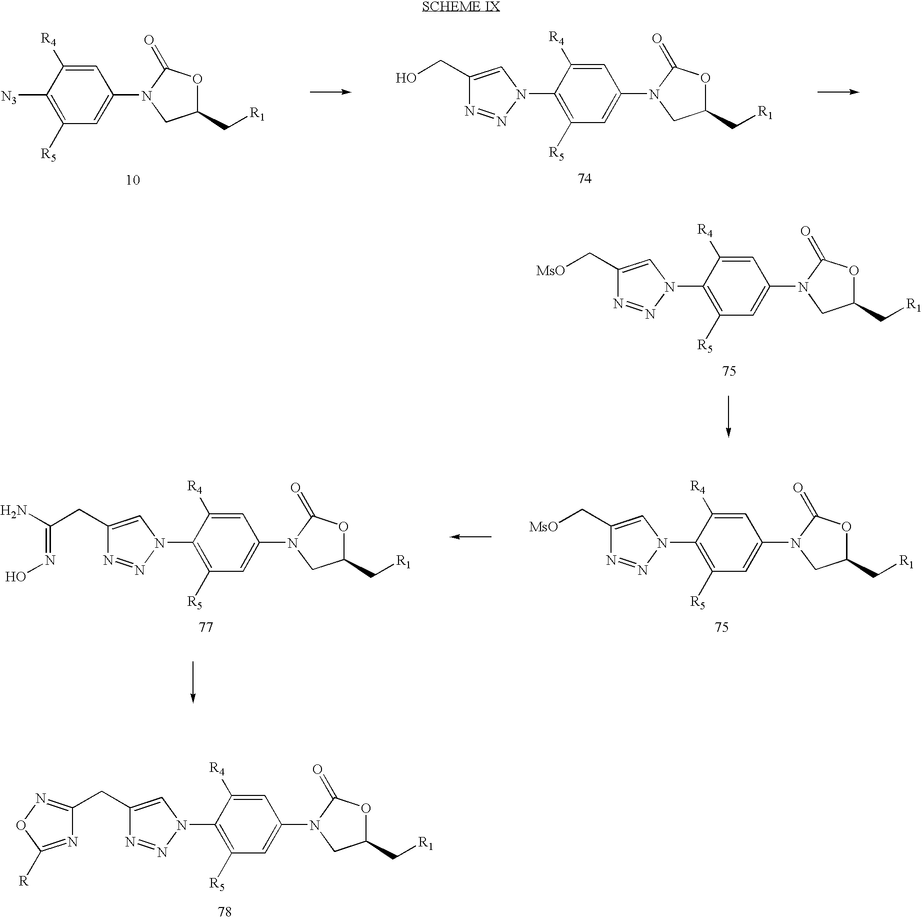

(i) reacting compounds of general formula 10, wherein R₁, R₄ and R₅ are same as explained in formula (I), in presence of reagent such as, but not limited with prop-2-yn-1-ol, cuprous iodide, N,N-diisopropylethylamine in presence of solvent such as, but not limited to dimethylformamide to obtain compounds of general formula 74.
(ii) reacting compounds of general formula 74 with reagent such as, but not limited to mesyl chloride, triethylamine to obtain compounds of general formula 75.
(iii) reacting compounds of general formula 75 with reagent such as, but not limited to potassium cyanide or sodium cyanide to obtain compounds of general formula 76.
(iv) reacting compounds of general formula 76 with reagent such as, but not limited to hydroxylamine to obtain compounds of general formula 77.
(v) reacting compounds of general formula 77 with reagent such as, but not limited to triethyl orthoformate, acetic anhydride to obtain compounds of general formula 78.

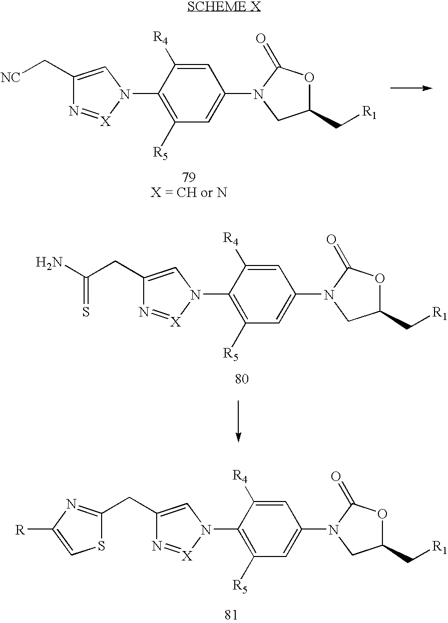

(i) reacting compounds of general formula 79, wherein X is CH or N and R₁, R₄ and R₅ are as defined in formula (I), with reagent such as, but not limited to hydrogen sulfide in presence of solvent, but not limited to dioxane, dimethylformamide to obtain compounds of general formula 80.
(ii) reacting compounds of general formula 80 with reagent such as, but not limited to RCOCH₂Cl to obtain compounds of general formula 81 wherein R is optionally substituted alkyl.

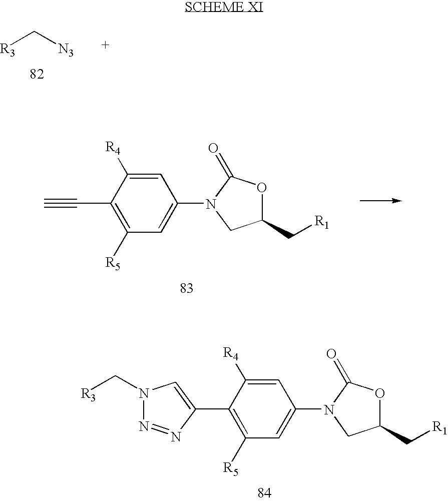

(i) reacting compounds of general formula 82, wherein R₃ is hetero aryl compounds, with general compounds of formula 83, wherein R₁, R₄ and R₅ are as defined in formula (I), in presence of reagent such as, but not limited with cuprous iodide, N,N-diisopropylethylamine in presence of solvent such as, but not limited to acetonitrile to obtain compounds of general formula 84.

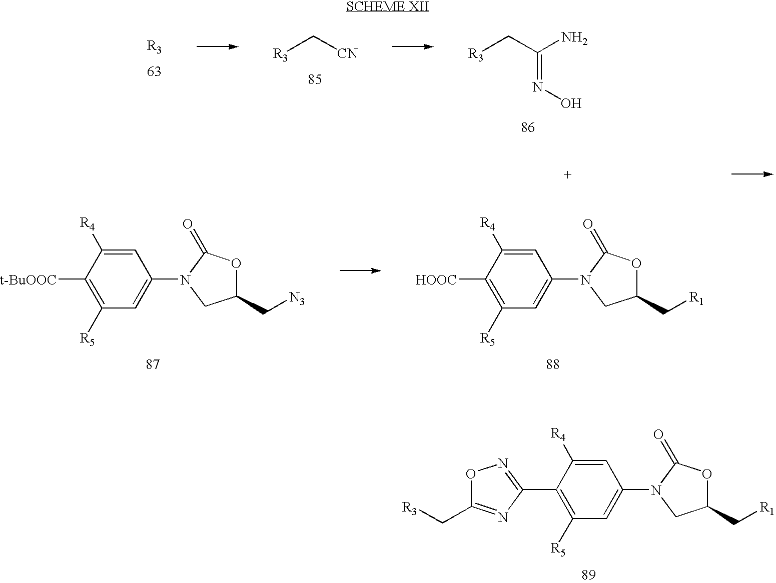

(i) reacting compounds of general formula 63, wherein R₃ is selected from heteroaryl such as triazole or pyridine, with reagent such as, but not limited to potassium carbonate, bromoacetonitrile to obtain compounds of general formula 85.
(ii) reacting compounds of general formula 85 with reagent such as, but not limited to hydroxylamine to obtain compounds of general formula 86.
(iii) reacting compounds of general formula 87, wherein R₄ and R₅ are as defined in formula (I), with reagent such as, but not limited to triphenylphosphane, water, tetrahydrofuran and acetic anhydride, pyridine to obtain compounds of general formula 88, wherein R₁ is as defined in formula (I).
(iv) reacting compounds of general formula 88 with compounds of general formula 86, with reagent such as, but not limited to N-ethyl-N′-(3-dimethylaminopropyl)carbodiimide (EDCI), hydroxybenzotriazole (HOBT), N,N- or dicyclohexylcarbodiimide (DCC), 2-(1H-7-azabenzotriazol-1-yl)-1,1,3,3-tetramethyl uronium hexafluorophosphate (HATU) in presence of solvent such as, but not limited to dimethylformamide to obtain compounds of general formula 89, wherein R₃ is selected from heteroaryl such as triazole or pyridine.

Any of the compounds of the following general formulae (I-i), (I-ii), (I-iii), (I-iv) & (I-v), wherein R₂, R₃, R₄, R₅ & A are as defined in the general formula (I) can be prepared by following any one or more of the similar methodologies described in the above schemes I-XII.

### Conversion-I:

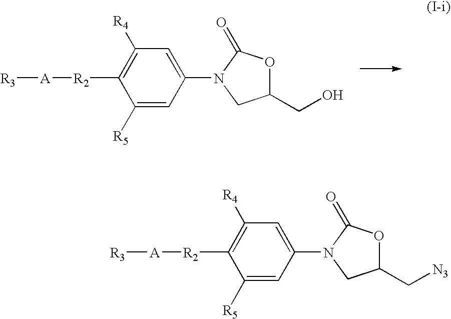

### Conversion-II:

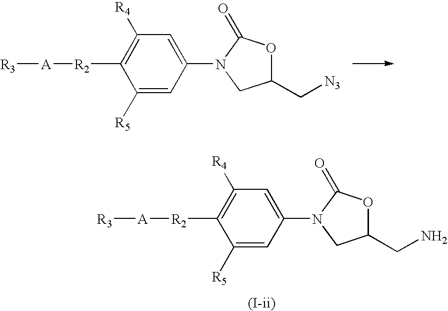

### Conversion-III:

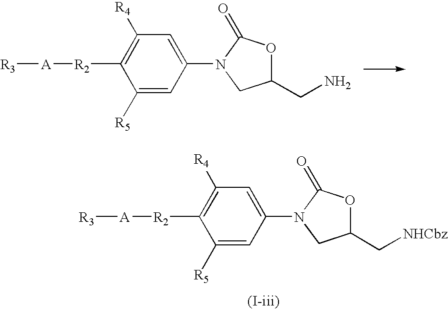

### Conversion-IV:

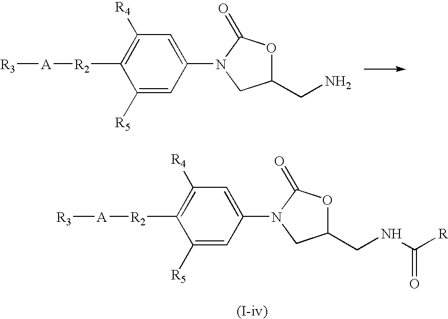

### Conversion-V:

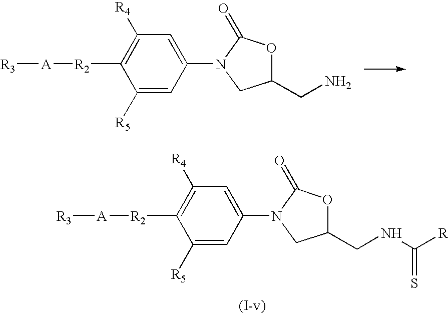

The novel compounds of the present invention were prepared according to the procedure of the following schemes and examples, using appropriate materials and are further exemplified by the following specific examples. The most preferred compounds of the invention are any or all of those specifically set forth in these examples. These compounds are not, however, to be construed as forming the only genus that is considered as the invention, and any combination of the compounds or their moieties may itself form a genus. The following examples further illustrate details for the preparation of the compounds of the present invention. Those skilled in the art will readily understand that known variations of the conditions and processes of the following preparative procedures can be used to prepare these compounds. All temperatures are degrees Celsius unless otherwise noted.

### Preparation 1

### 2-Fluoro-4-nitro-benzoic acid

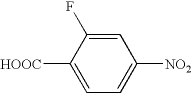

A solution of 2-fluoro-4-nitro toluene (50 grams, 322.0 mmol) dissolved in a mixture of acetic acid (625 mL) and concentrated sulfuric acid (157 mL), was treated with an aqueous solution of chromium trioxide (116 grams, 1160.0 mmol, in 100 mL water at 100° C.) for 3 hours. The reaction mixture was poured into ice-cold water (2 Litres) and extracted with diethyl ether (2×1 Litres). Evaporation of the volatiles left a residue, which was dissolved in 10% aqueous potassiumcarbonate solution (1 Litre) and extracted with diethyl ether (300 mL). The aqueous layer was acidified with diluted hydrogen chloride and the solid obtained was filtered and dried.

Yield: 66%,

MS (m/z): 186 (M⁺+1),

¹H NMR (300 MHz, CDCl₃): δ 8.24 (dd, J=1.6 & 7.0 Hz, 1H), 8.14-8.04 (m, 2H).

### Preparation 2

### Tert-butyl 2-fluoro-4-nitro-benzoate

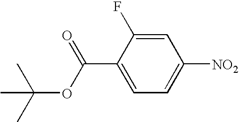

To a solution of 2-fluoro-4-nitrobenzoic acid (12.0 grams, 64.9 mmol) in dichloromethane, triethylamine (26 mL, 194.6 mmol) and 4-(dimethylamino) pyridine (2.37 grams, 19.5 mmol) were added followed by the addition of di-tert-butyl dicarbonate (22.5 mL, 97.0 mmol) at 10° C. The resulting mixture was stirred at room temperature for 2 hours. Solvent was evaporated and the residue obtained was dissolved in ethyl acetate (300 mL), washed with water (2×150 mL), 5% citric acid solution (2×150 mL) and brine solution (150 mL). Finally the organic layer was dried over anhydrous sodium sulfate and volatiles were evaporated.

Yield: 98%,

¹H NMR (300 MHz, CDCl₃): δ 8.05-8.03 (m, 2H), 8.01-7.96 (m, 1H), 1.63 (s, 9H).

### Preparation 3

### 2-Fluoro-4-nitro-phenyl-hydrazine

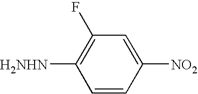

To a solution of 1,2-difluoro-4-nitro-benzene (25 grams, 157.2 mmol) in ethanol was added hydrazine hydrate (15.72 grams, 314.5 mmol) drop wise at 80° C. It was stirred for 2 hours at the same temperature. Ethanol was removed in rotavapor and the solid obtained was filtered and triturated in diethyl ether. The free flowing solid obtained after decanting the supernatant liquid was dried under high vacuum to obtain 26.7 grams of title compound.

Yield: 99.2%,

MS (m/z): 172 (M⁺+1),

¹H NMR (300 MHz, CDCl₃): δ 8.05 (dd, J=2.5 & 9.1 Hz, 1H), 7.89 (dd, J=2.5 & 11.8 Hz, 1H), 7.24 (t, J=8.7 Hz, 1H), 6.01 (bs, 1H), 3.75 (s, 2H).

### Preparation 4

### Ethyl 1-(2-fluoro-4-nitro-phenyl)-1H-pyrazole-4-carboxylate

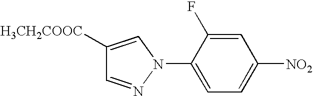

To a solution of 2-fluoro-4-nitro-phenyl)-hydrazine (17.0 grams, 99.4 mmol) in ethanol (350 mL) were added ethyl 2-formyl-3-oxo-propionate¹ (18.6 grams, 129.2 mmol) and sodium acetate (8.2 grams, 99.4 mmol) and stirred at room temperature for half an hour and then at 80° C. for 3 hours. Ethanol was removed in rotavapor and the residue obtained was dissolved in ethyl acetate. Ethyl acetate portion was washed successively with water (100 mL×2), saturated sodium bicarbonate solution (50 mL) and brine (100 mL). Organic layer was then dried over anhydrous sodium sulfate and concentrated to obtain a brown solid. Triturated with diethyl ether and filtered to get 13.6 grams of free flowing solid.

Yield: 49%,

MS (m/z): 280 (M⁺+1),

¹H NMR (300 MHz, CDCl₃): δ 8.64 (d, J=2.5 Hz, 1H), 8.18-8.30 (m, 4H), 4.36 (q, J=7.1 Hz, 2H), 1.39 (t, J=7.1 Hz, 3H).

### Preparation 5

### [1-(2-Fluoro-4-nitro-phenyl)-1H-imidazol-4-yl]methanol

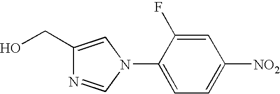

A mixture of 1,2-difluoronitrobenzene (22 grams, 138.4 mmol), diisopropylethylamine (150 mL) and 4-hydroxymethyl imidazole (18.62 grams, 138.4 mmol) was heated to 100° C. for 3 hours. The bottom layer was separated from the two layers formed and water was added to obtain yellow solid. It was filtered, dried and washed with 10% acetone in hexane.

Yield: 91%,

¹H NMR (CDCl₃, 300 MHz): δ 8.15-8.25 (m, 2H), 7.94 (m, 1H), 7.61 (m, 1H), 7.32 (s 1H), 4.72 (s, 2H).

### Preparation 6

### 4-(Tert-butyl-dimethyl-silanyloxymethyl)-1-(2-fluoro-4-nitro-phenyl)-1H-imidazole

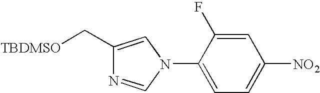

Tert-Butyldimethylsilyl chloride (4.76 grams, 31 mmol) was added to a mixture of [1-(2-fluoro-4-nitro-phenyl)-1H-imidazol-4-yl]-methanol (5 grams, 20.6 mmol) and imidazole (2.85 grams, 41.2 mmol) in dimethylformamide (50 mL) at 10-15° C. in portions. The resulting mixture was stirred at room temperature for 16 hours. Dimethylformamide was distilled under vacuum and to the residue obtained was added water. The solid obtained was filtered and dried.

Yield: 61%,

¹H NMR (CDCl₃, 300 MHz): δ 8.16-8.18 (m, 2H), 7.89-7.90 (m, 1H), 7.51-7.62 (m, 1H), 7.26 (s, 1H), 4.77 (s, 2H), 0.94 (s, 9H), 0.11 (s, 6H).

### Preparation 7

### Benzyl-(4-nitro-2-fluoro-phenyl)-amine

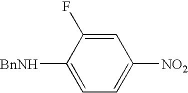

To a suspension of 3,4-difluoronitrobenzene (10.0 grams, 63.0 mmol) in dimethylformamide (100 mL) was added N-ethyl diisopropylamine (12.2 grams, 94.5 mmol) drop wise at room temperature followed by the addition of benzylamine (8.1 grams, 76.6 mmol). Reaction mixture was stirred overnight at room temperature and then poured into cold water with continuous stirring. Fine solids obtained were filtered & dried.

Yield: 70%,

IR (KBr, cm⁻¹): 3354, 1612, 1549, 1493, 1288, 1185, 1068, 876, 810, 732, 542,

MS (m/z): 247 (M⁺), 246, 91,

¹H NMR (CDCl₃, 200 MHz): δ 7.98-7.86 (m, 2H), 7.42-7.25 (m, 5H), 6.67-6.58 (m, 1H) 5.08 (bs, 1H), 4.50-4.46 (m, 2H).

### Preparation 8

### N-Hydroxy-2-[1,2,4]triazol-1-yl-acetamidine

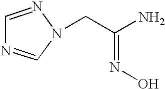

To a solution of [1,2,4] Triazol-1-yl-acetonitrile (1.5 grams, 13.9 mmol) in ethanol were added sodium carbonate (4.4 grams, 41.7 mmol) and hydroxylamine hydrochloride (3.86 grams, 55.5 mmol). The mixture was heated to 80° C. for overnight. The reaction mixture was concentrated and extracted with ethyl acetate and purified by column to get the desired product (1.9 grams).

Yield: 97%

¹H NMR (200 MHz, CDCl₃+DMSO): δ 9.34 (bs, 1H), 8.51 (s, 1H), 7.95 (s, 1H), 5.58 (bs, 2H), 4.74 (s, 2H).

ES-MS (m/z): 142 (M⁺+1)

### Preparation 9

### 2-(Pyridin-2-yl)acetohydrazide

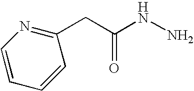

To a solution of Ethyl-2-(pyridine-2-yl)acetate (2.3 grams, 11.5 mmol) in ethanol hydrazine hydrate (2 mL) was added and the reaction mixture was refluxed for 3 hours. The solvent was co-evaporated with toluene to obtain the product as white solid 1.2 grams.

Yield: 71%

### Preparation 10

### Tert-butyl 4-amino-2-fluoro-benzoate

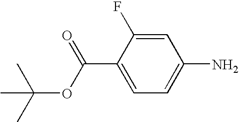

Tert-Butyl-2-Fluoro-4-nitro-benzoate (15 grams, 62.2 mmol) was added to a solution of ammonium chloride (34 grams, 622.0 mmol) in a mixture of water (45 mL) and ethanol (90 mL) at 95° C., followed by the addition of iron powder (10.5 grams, 187 mmol) in three lots. Stirring was continued at that temperature for 1 hour. The solids were filtered off in hot condition and the filtrate was evaporated. The residue obtained was dissolved in water (100 mL) and extracted with diethyl ether (2×150 mL). The organic layer was dried over sodium sulfate and the volatiles were evaporated.

Yield: 98%,

MS (m/z): 210 (M⁺−1),

¹H NMR (300 MHz, CDCl₃): δ 7.70 (t, J=8.4 Hz, 1H), 6.45 (d, J=8.5 Hz, 1H), 6.37 (dd, J=1.6 & 11.0 Hz, 1H), 1.58 (s, 9H).

### Preparation 11

### Benzyl-[5-aminomethyl-2-oxo-oxazolidin-3-yl)-2-fluoro-phenyl]-carbamate

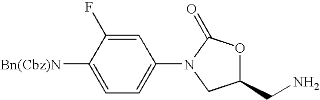

To a solution of benzyl-(4-[5-azidomethyl-2-oxo-oxazolidin-3-yl)-2-fluoro-phenyl]-carbamate (6 grams, 12.6 mmol) in tetrahydrofuran (50 mL) was added triphenylphosphine in small batches (3.64 grams, 14 mmol) and stirred for 4 hours. The reaction mixture was then heated to 40-50° C. for 16 hours after the addition of water (1 mL). Solvent was evaporated and the residue obtained was purified by column chromatography on silica gel using methanol and chloroform (1:9) as eluent to obtain the title compound.

Yield: 70%,

IR (KBr, cm⁻¹): 3334, 2926, 2606, 2497, 1751, 1655, 1524, 1475, 1441, 1226, 1081, 1037, 850, 753, 591,

MS (m/z): 450 (M⁺+1), 386, 376, 279,

¹H NMR (300 MHz, CDCl₃): δ 7.52-6.58 (m, 14H), 5.15 (s, 2H), 4.80 (s, 2H), 4.78-4.58 (m, 1H), 3.93 (t, J=8.7 Hz, 1H), 3.81 (t, J=7.8 Hz, 1H), 3.16-2.90 (m, 2H),

### Preparation 12

### Pyrazol-1-yl-acetonitrile

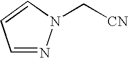

To a solution of pyrazole (10 grams, 14.7 mmol) in dry dimethylformamide (150 mL) was added anhydrous potassium carbonate and stirred at room temperature for 10 minutes. Bromo acetonitrile (40 mL, 58.8 mmol) was then added and stirred at ice temperature. The reaction mixture was then allowed to stir at room temperature overnight. Potassium carbonate was filtered off and the filtrate concentrated at reduced pressure. The resulting mass was then diluted with ethyl acetate (500 mL) and the ethyl acetate layer was washed with brine solution and dried over sodium sulphate. Volatiles were evaporated and the residue obtained was purified by column chromatography over silica gel using ethyl acetate and petroleum ether (1:4) as eluent. Title compound was obtained as light yellow liquid.

Yield: 69%,

IR (neat, cm⁻¹): 3250, 3126, 2988, 1663, 1518, 1395, 1288 and 757,

MS (m/z): 107 (M⁺+1),

¹H NMR (200 MHz, CDCl₃): δ 7.61-7.60 (m, 1H), 7.57-7.55 (m, 1H), 5.11 (s, 2H).

### Preparation 13

### N-[3-(4-Acetyl-3-fluoro-phenyl)-2-oxo-oxazolidin-5-ylmethyl]-acetamide

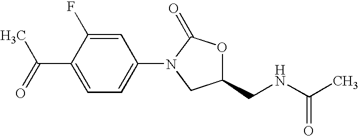

The compound has been synthesized according to the reported procedure (J. Med. Chem. 1992, 35, 1156-1165)

### Preparation 14

### N-{3-[4-(2-Bromo-acetyl)-3-fluoro-phenyl]-2-oxo-oxazolidin-5-ylmethyl}-acetamide

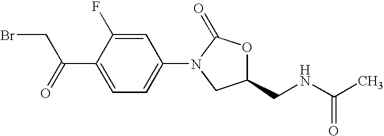

To a solution of N-[3-(4-acetyl-3-fluoro-phenyl)-2-oxo-oxazolidin-5-ylmethyl]-acetamide (450 μL, 1.53 mmol) in methanol (5 mL) was added bromine (0.12 mL, 2.29 mmol) at 0° C. and stirred for 5 hours. The temperature was slowly increased from 0° C. to room temperature. Water (1 mL) was added and the reaction mixture was allowed to stir at room temperature for additional 1 hour. Evaporation of methanol gave white color solid.

Yield: 55%,

IR (KBr, cm⁻¹): 3282, 2919, 1733, 1626, 1414, 1197, 1054, 862 and 752,

MS (m/z): 375 (M⁺+2), 295,

¹H NMR (200 MHz, CDCl₃): δ 7.96 (t, J=8.4 Hz, 1H), 7.60 (dd, J=2.0 & 11.7 Hz, 1H), 7.27-7.21 (m, 1H), 6.17 (bs, 1H), 4.89-4.77 (m, 1H), 4.48 (d, J=2.4 Hz, 2H), 4.13-4.04 (m, 1H), 3.87-3.79 (m, 1H), 3.71-3.65 (m, 2H), 2.02 (s, 3H).

### Preparation 15

### Benzyl {4-[5(S)-(acetylamino methyl)-2-oxo-oxazolidin-3-yl]-2-fluoro-phenyl}-benzyl-carbamate

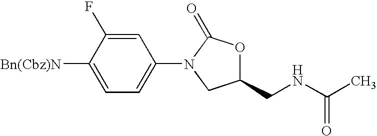

Benzyl-[4-(5-azidomethyl-2-oxo-oxazolidin-3-yl)-2-fluoro-phenyl]-carbamate (650 mg, 13.7 mmol) was treated with thiolacetic acid (1.5 mL) at room temperature for 24 hours. Excess solvent was evaporated and the residue obtained was purified by column chromatography on silica gel using methanol and chloroform (1:9) as eluent to obtain the title compound as white solid.

Yield: 67%.

Alternatively, a solution of benzyl-[5-aminomethyl-2-oxo-oxazolidin-3-yl)-2-fluoro-phenyl]-carbamate (3 grams, 6.68 mmol) and pyridine (1.1 mL, 13.36 mmol) in chloroform (75 mL) was treated with acetic anhydride (0.96 mL, 10.02 mmol) at 0° C. and stirred at room temperature for half an hour. The reaction mixture was extracted with chloroform (3×100 mL) after the addition of water (200 mL). The combined organic layer was washed with brine, dried over sodium sulfate and concentrated. The residue obtained was purified by column chromatography on silica gel using methanol and chloroform (1:9) as eluent to obtain the title compound as white solid.

Yields: 52%,

IR (KBr, cm⁻¹): 3323, 1755, 1707, 1520, 1403, 1218, 1125,

MS (m/z): 492 (M⁺+1), 448, 428, 391, 384, 351, 314,

¹H NMR (CDCl₃, 200 MHz): δ 7.50-7.01 (m, 13H), 6.20 (bs, 1H), 5.15 (s, 2H), 4.80-4.70 (m, 3H), 3.75-3.57 (m, 3H), 1.99 (s, 3H).

### Preparation 16

### N-[3-(4-Amino-3-fluoro-phenyl)-2-oxo-oxazolidin-5(S)-yl-methyl]-acetamide

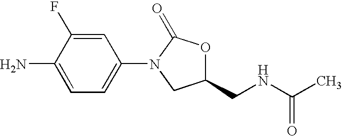

A solution of benzyl {4-[5-(acetylamino methyl)-2-oxo-oxazolidin-3-yl]-2-fluoro-phenyl}-benzyl-carbamate (200 mg, 0.41 mmol) in methanol (25 mL) was treated with 10% palladium on charcoal (84 mg, 0.80 mmol) under hydrogen atmosphere for 16 hours. The catalyst was filtered over a pad of celite and the filtrate was concentrated. The residue obtained was purified by column chromatography on silica gel using methanol and chloroform (1:9) as eluent to obtain the title compound as white solid.

Yield: 61%,

IR (KBr, cm⁻¹): 3436, 3356, 2926, 2855, 1733, 1664, 1522, 1420, 1228,

MS (m/z): 268 (M⁺+1),

¹H NMR (DMSO-d₆, 400 MHz): δ 8.20 (t, J=5.6 Hz, 1H), 7.31 (dd, J=3.5 & 10.0 Hz, 1H), 6.95 (dd, J=2.4 & 8.8 Hz, 1H), 6.76 (dd, J=8.8 & 9.9 Hz, 1H), 5.0 (s, 2H), 4.70-4.62 (m, 1H), 4.01 (t, J=8.8 Hz, 1H), 3.64 (dd, J=6.4 & 9.1 Hz, 1H), 3.38 (t, J=8.6 Hz, 2H), 1.86 (s, 3H).

### Preparation 17

### N-[3-(4-Azido-3-fluoro-phenyl)-2-oxo-oxazolidin-5(S)-yl-methyl]-acetamide

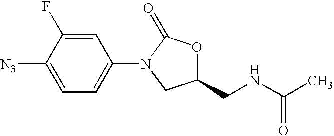

To a solution of N-[3-(4-amino-3-fluoro-phenyl)-2-oxo-oxazolidin-5-yl-methyl]-acetamide (200 mg, 0.75 mmol) in 50% aqueous hydrogen chloride (20 mL) was added sodium nitrite (104 mg, 1.5 mmol) in small batches at 5-10° C. An aqueous solution of sodium azide (98 mg, 1.5 mmol) and sodium acetate (1.28 grams, 15 mmol) was added after 0.5 hours and stirring was continued for 15 minutes. Reaction mixture was then diluted with water & extracted with ethyl acetate (2×50 mL). Ethyl acetate layer was washed with brine and dried over sodium sulfate and concentrated. The residue obtained was purified by column chromatography on silica gel using methanol and chloroform (1:9) as eluent to obtain the title compound as light brown solid.

Yield: 59%,

IR (KBr, cm⁻¹): 3266, 3075, 2925, 2130, 1745, 1653, 1516, 1415, 1308,

MS (m/z): 294 (M⁺+1), 281, 268, 253,

¹H NMR (DMSO-d₆, 400 MHz): δ 8.21 (t, J=5.6 Hz, 1H); 7.58 (dd, J=2.4 & 12.3 Hz, 1H); 7.36-7.16 (m, 2H); 4.76-4.70 (m, 1H); 4.11 (t, J=8.8 Hz, 1H); 3.73 (dd, J=6.4 & 9.1 Hz, 1H); 3.41 (t, J=5.6 Hz, 2H); 1.83 (s, 3H).

### Preparation 18

### 2-Pyrazol-1-yl-thioacetamide

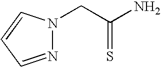

To a solution of pyrazol-1-yl-acetonitrile (10 grams, 93.5 mmol) in ethanol was added triethyl amine (19 mL, 140 mmol). Hydrogen sulphide (H₂S) gas was then passed through reaction mixture for 5 hours at room temperature. The organic solvent was evaporated and the residue was purified by column chromatography over silica gel using ethyl acetate and petroleum ether (1:3) as eluent. Title compound was obtained as light yellow solid.

Yield: 62%,

IR (KBr, cm⁻¹): 3301, 3141, 2965, 1732, 1636, 973 and 740,

MS (m/z): 142 (M⁺+1),

¹H NMR (200 MHz, CDCl₃): δ 7.97 (bs, 2H), 7.64 (d, J=2.0 Hz, 1H), 7.50 (d, J=2.0 Hz, 1H), 6.37 (t, J=2.0 Hz, 1H), 5.21 (s, 2H).

### Preparation 19

### Ethyl 1-(4-amino-2-fluoro-phenyl)-1H-pyrazole-4-carboxylate

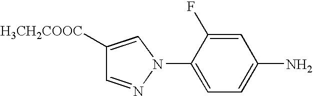

To a solution of ethyl 1-(2-fluoro-4-nitro-phenyl)-1H-pyrazole-4-carboxylate (35.2 grams, 126.2 mmol) and ammonium chloride (67.5 grams, 1261.6 mmol) in a mixture of ethanol and water (2:1, 400 mL) was added iron powder (21.19 grams, 378.5 mmol) in small batches. It was stirred for 1.5 hours at the same temperature. Reaction mixture was then filtered in hot condition and solid was washed with ethyl acetate. Filtrate was concentrated and the residue obtained was dissolved in diethyl ether (500 mL). The ether layer was washed with brine solution (200 mL) and concentrated to obtain 31.4 grams of light yellow solid.

Yield: 99%,

MS (m/z): 250 (M⁺+1),

¹H NMR (300 MHz, CDCl₃): δ 8.27 (d, J=2.3 Hz, 1H), 8.07 (s, 1H); 7.51 (t, J=8.7 Hz, 1H), 6.48-6.53 (m, 2H), 4.32 (q, J=7.1 Hz, 2H), 3.94 (s, 2H), 1.36 (t, J=7.1 Hz, 3H).

### Preparation 20

### 4-[4-(Tert-butyl-dimethyl-silanyloxymethyl)-imidazol-1-yl]-3-fluoro-phenylamine

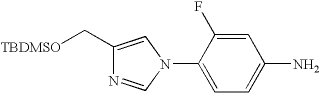

Ammonium chloride (53 grams, 53.5 mmol) and 4-(tert-butyl-dimethyl-silanyloxymethyl)-1-(2-fluoro-4-nitro-phenyl)-1H-imidazole (35 grams, 97 mmol) were added to a mixture of ethanol and water (2:1, 315 mL) and heated to 60° C. for 5 minutes. Iron powder (16.62 grams, 291 mmol) was added in portions and after the completion of addition the reaction mixture was heated to 95° C. for 5 hours. Inorganic material was filtered over a pad of celite and the filtrate was concentrated. The residue obtained was extracted with diethyl ether, which upon concentration afforded the product.

Yield: 97.2%,

¹H NMR (CDCl₃, 300 MHz): δ 7.59-7.60 (m, 1H), 7.03-7.13 (m, 2H), 6.45-6.53 (m, 2H), 4.77 (s, 2H), 4.00 (s, 2H), 0.95 (s, 9H), 0.11 (s, 6H).

### Preparation 21

### N′1′-Benzyl-2-fluoro-benzene-1,4-diamine

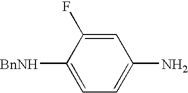

To a solution of benzyl-(4-nitro-2-fluoro-phenyl)-amine (5.0 grams, 20.3 mmol) in methanol (70 mL) was added nickel chloride hexahydrate (9.5 grams, 42.86 mmol) and cooled to 0° C. Sodium borohydride (2.35 grams, 61 mmol) was added to the reaction mixture in small batches and stirred at 5-10° C. for 1 hour. The reaction mixture was extracted with ethyl acetate (2×250 mL) after the addition of water. Ethyl acetate layer was washed with brine, dried over sodium sulfate and concentrated. Purification of the residue by column chromatography over silica gel using methanol:chloroform (1:9) gave the title compound as a brown liquid.

Yield: 68%,

IR (Neat, cm⁻¹): 3361, 3030, 1522, 1219, 956, 802, 732, 668, 597,

MS (m/z): 217 (M⁺+1), 216,

¹H NMR (CDCl₃, 200 MHz): δ 7.34-7.24 (m, 5H), 6.57-6.31 (m, 3H), 4.27 (s, 2H), 3.63-3.50 (m, 2H).

### Preparation 22

### Tert-butyl 4-benzyloxycarbonylamino-2-fluoro-benzoate

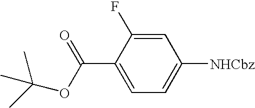

To a stirred solution of tert-butyl 4-amino-2-fluoro-benzoate (13 grams, 61.6 mmol) and sodium bicarbonate (21 grams, 246 mmol) in tetrahydrofuran (200 mL), benzyloxy carbonyl chloride (CBz-Cl) (43 mL) was added at 0° C. The resulting mixture was stirred at room temperature for 16 hours and later it was heated to 50° C. for 3 hours. Solids were filtered off and filtrate was evaporated. The residue obtained was washed with hexane (100 mL) and dried.

Yield: 98%,

¹H NMR (300 MHz, CDCl₃): δ 7.81 (t, J=8.4 Hz, 1H), 7.43-7.35 (m, 5H), 7.03 (dd, J=2.0 & 8.6 Hz, 1H), 6.93 (bs, 1H), 5.21 (s, 2H), 1.60 (s, 9H).

### Preparation 23

### 4-Benzyloxycarbonylamino-2-fluoro-benzoic acid

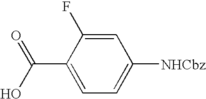

To a solution of tert-butyl 4-benzyloxycarbonylamino-2-fluoro-benzoate (21.0 grams, 60.9 mmol) in dichloromethane (100 mL), trifluoroaceticacid (100 mL) was added drop wise at 0° C. and stirred at room temperature for 1 hour. Solvent was evaporated and the residue obtained was washed with diethyl ether (100 mL) to obtain pale yellow solid.

Yield: 92%,

MS (m/z): 288 (M⁺−1),

¹H NMR (400 MHz, CDCl₃+DMSO-d₆): δ 12.20 (hump, —OH), 7.83 (t, J=8.6 Hz, 1H), 7.22-7.58 (m, 7H), 5.20 (s, 2H).

### Preparation 24

### Methyl 2-(4-benzyloxycarbonylamino-2-fluoro-benzoylamino)-3-hydroxy-propionate

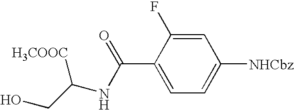

To a solution of 4-benzyloxycarbonylamino-2-fluoro-benzoic acid (15.7 grams, 54.3 mmol) in dry tetrahydrofuran (400 mL), 1-(3-Dimethylaminopropyl)-3-ethylcarbodiimide hydrochloride (12.5 mL, 70.6 mmol), hydroxybenzotriazole (8.8 grams, 65.2 mmol), L-Serine methyl ester hydrochloride (12.6 grams, 81.5 mmol) and diisopropylethylamine (28 mL, 217.0 mmol) were added at room temperature and stirred for 1 hour. Solvent was evaporated and the residue obtained was dissolved in ice-cold water. Aqueous layer was extracted with ethyl acetate (250 mL×2) and the total organic layer was washed with 5% potassium carbonate solution followed by brine solution. Finally it was dried over sodium sulphate and volatiles were evaporated to obtain pale yellow solid.

Yield: 85%,

MS (m/z): 389 (M⁺−1),

¹H NMR (300 MHz, CDCl₃): δ 7.99 (t, J=8.6 Hz, 1H), 7.52-7.68 (m, 2H), 7.40 (bs, 5H), 6.97-7.08 (m, 2H), 4.85 (bs, 1H), 4.05 (s, 2H), 3.58 (s, 3H), 2.58 (bs, 1H).

### Preparation 25

### Methyl 2-(4-benzyloxycarbonylamino-2-fluoro-phenyl)-4,5-oxazole-4-carboxylate

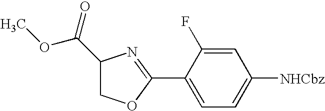

A solution of methyl 2-(4-benzyloxycarbonylamino-2-fluoro-benzoylamino)-3-hydroxy-propionate (6.0 grams, 15.4 mmol) in dry tetrahydrofuran (125 mL) was treated with Burgess reagent (4.0 grams, 16.9 mmol) at 75° C. for 3 hours. Solvent was evaporated and organic compound was extracted with ethyl acetate. It was washed with water, 5% potassium carbonate solution and saturated brine solution successively. Finally it was dried over anhydrous sodium sulphate and volatiles were evaporated. The residue obtained was purified by column chromatography (60-120 mesh silica gel) by using 25% ethyl acetate:hexane solvent system.

Yield: 72%,

MS (m/z): 373 (M⁺+1),

¹H NMR (300 MHz, CDCl₃): δ 7.85 (t, J=8.2 Hz, 1H), 7.46 (d, J=13.3 Hz, 1H), 7.40 (s, 5H), 7.08 (d, J=6.1 Hz, 2H), 5.20 (s, 2H), 4.95 (t, J=9.2 Hz, 1H), 4.68-4.52 (m, 2H).

### Preparation 26

### Benzyl [3-fluoro-4-(4-hydroxymethyl-oxazol-2-yl)-phenyl]-carbamate

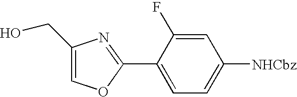

Methyl-2-(4-benzyloxycarbonylamino-2-fluoro-phenyl)-oxazole-4-carboxylate (4.5 grams, 12.2 mmol) was added to a suspension of lithium aluminium hydride (925 mg, 24.3 mmol) in tetrahydrofuran (110 mL) at 0° C. and stirred for 10 minutes. The reaction was quenched by the addition of water at cold condition and stirred for 30 minutes. Solids were filtered off and the filtrate was concentrated to obtain a pale yellow solid.

Yield: 72%,

MS (m/z): 341 (M⁺−1),

¹H NMR (300 MHz, DMSO-d₆): δ 8.03 (s, 1H), 8.92 (t, J=8.6 Hz, 1H), 7.55 (d, J=13.6 Hz, 1H), 7.43-7.32 (m, 6H), 5.27 (t, J=5.6 Hz, 1H), 5.20 (s, 2H), 4.43 (d, J=5.5 Hz, 2H).

### Preparation 27

### Benzyl {4-[4-(tert-butyl-dimethyl-silanyloxymethyl)-oxazol-2-yl]-3-fluoro-phenyl}-carbamate

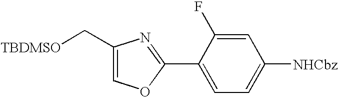

To a solution of benzyl [3-fluoro-4-(4-hydroxymethyl-oxazol-2-yl)-phenyl]-carbamate (4.5 grams, 13.2 mmol) and imidazole (1.8 grams, 26.3 mmol) in dimethylformamide (45 mL), tert-Butyldimethylsilyl chloride (3.0 grams, 19.7 mmol) was added at 0° C. and stirred at room temperature for 30 minutes. Solvent was evaporated and the residue obtained was dissolved in water. It was extracted with ethyl acetate and dried over anhydrous sodium sulfate. The volatiles were evaporated to obtain a pale yellow solid.

Yield: 84%,

¹H NMR (300 MHz, CDCl₃): δ 7.93 (t, J=8.4 Hz, 1H), 7.61 (s, 1H), 7.51 (dd, J=1.8 & 11.1 Hz, 1H), 7.43-7.34 (m, 5H), 7.09 (dd, J=2.0 & 6.6 Hz, 1H), 6.82 (s, 1H), 5.21 (s, 2H), 4.65 (s, 2H), 0.93 (s, 9H), 0.06 (s, 6H).

### Preparation 28

### Ethyl [1-(4-benzyloxycarbonylamino-2-fluoro-phenyl)-1H-pyrazole-4-carboxylate

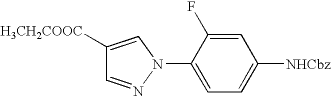

To a solution of ethyl 1-(4-amino-2-fluoro-phenyl)-1H-pyrazole-4-carboxylate (32.65 grams, 131.14 mmol) in tetrahydrofuran (800 mL) was added sodium bicarbonate (44.06 grams, 524.56 mmol) followed by the addition of benzylchloroformate (56.13 mL, 393.42 mmol) drop wise at ice temperature. Stirring was continued overnight at room temperature. Reaction mixture was then filtered and the solid obtained was washed with tetrahydrofuran. The filtrate was concentrated to obtain a semisolid, which was stirred in hexane for 15 minutes to obtain 42.0 grams of light yellow solid.

Yield: 83%,

MS (m/z): 384 (M⁺+1),

¹H NMR (300 MHz, CDCl₃): δ 8.41 (d, J=2.4 Hz, 1H), 8.10 (s, 1H), 7.77 (t, J=8.7, 1H) 7.64 (d, J=11.3 Hz, 1H), 7.35-7.42 (m, 4H), 7.08 (dt, J=1.1 & 8.8 Hz, 1H), 6.90 (s, 1H), 5.22 (s, 2H), 4.34 (q, J=7.1 Hz, 2H), 1.37 (t, J=7.1 Hz, 3H).

### Preparation 29

### Benzyl [3-fluoro-4-(4-hydroxymethyl-pyrazol-1-yl)-phenyl]-carbamate

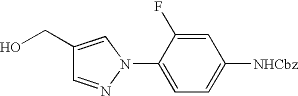

To a suspension of lithium aluminum hydride (1.9 grams, 52.22 mmol) in dry tetrahydrofuran (250 mL) was added ethyl 1-(4-benzyloxycarbonylamino-2-fluoro-phenyl)-1H-pyrazole-4-carboxylate (10.0 grams, 26.11 mmol) in small batches at 0° C. and stirring was continued for additional one hour at the same temperature. Reaction mixture was quenched by adding 10% potassium carbonate solution (20 mL). The filtrate obtained after filtration was concentrated. The residue thus obtained was dissolved in ethyl acetate, washed with water (150 mL×2) and brine solution (100 mL×1) successively. Organic layer was then dried over anhydrous sodium sulfate and concentrated to obtain 7.76 grams of yellow color product.

Yield: 87.7%,

MS (m/z): 342 (M⁺+1),

¹H NMR (300 MHz, CDCl₃): δ 7.94 (d, J=2.4 Hz, 1H), 7.72-7.78 (m, 2H), 7.61 (d, J=11.0 Hz, 1H), 7.37-7.40 (m, 4H), 7.05 (d, J=8.4 Hz, 1H), 6.84 (s, 1H), 5.21 (s, 2H), 4.67 (bs, 2H).

### Preparation 30

### Benzyl {4-[4-(tert-butyl-dimethyl-silanoyloxymethyl)-pyrazol-1-yl]-3-fluoro-phenyl}-carbamate

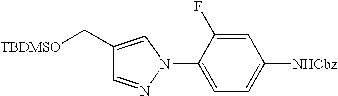

The title compound is prepared by following the procedure as described in preparation 27, by taking appropriate starting materials.

Yield: 83.17%,

MS (m/z): 456 (M⁺+1),

¹H NMR (300 MHz, CDCl₃): δ 7.86 (d, J=2.7 Hz, 1H), 7.75 (t, J=8.7 Hz, 1H), 7.65 (s, 1H), 7.60 (d, J=13.5 Hz, 1H), 7.35-7.41 (m, 4H), 7.05 (d, J=8.8 Hz, 1H), 6.78 (s, 1H), 5.21 (s, 2H), 4.72 (s, 2H), 0.95 (s, 9H), 0.18 (s, 6H).

### Preparation 31

### Benzyl {4-[4-(tert-butyl-dimethyl-silanyloxy)-imidazol-1-yl]-3-fluoro-phenyl}carbamate

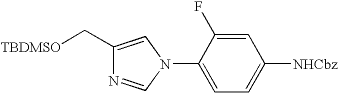

The title compound is prepared by following the procedure as described in preparation 28, by taking appropriate starting materials.

Yield: 75.5%,

¹H NMR (300 MHz, CDCl₃): δ 7.10-7.82 (m, 10H), 5.21 (s, 2H), 4.75 (s, 2H), 0.95 (s, 9H), 0.15 (s, 6H).

### Preparation 32

### Benzyl 4-(benzyl-benzyloxycarbonyl-amino)-3-fluoro-phenyl)-carbamate

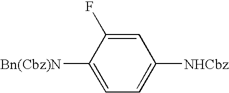

The title compound is prepared by following the procedure as described in preparation 28, by taking appropriate starting materials.

Yield: 45%.

MS (m/z): 485 (M⁺+1), 441, 377, 351,

IR (KBr, cm⁻¹): 3315, 3033, 2930, 1693, 1536,

¹H NMR (CDCl₃, 200 MHz): δ 7.36-7.22 (m, 17H); 6.05 (bs, 2H); 5.17 (s, 4H), 4.77 (s, 2H).

### Preparation 33

### Prep 3-{4-[4-(Tert-butyl-dimethyl-silanyloxymethyl)-oxazol-2-yl]-3-fluoro-phenyl}-5-(R)-hydroxymethyl-oxazolidin-2-one

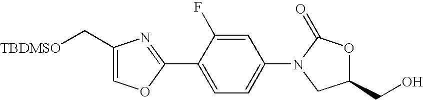

To a solution of benzyl {4-[4-(tert-butyl-dimethyl-silanyloxymethyl)-oxazol-2-yl]-3-fluoro-phenyl}-carbamate (5.0 grams, 10.9 mmol) in dry tetrahydrofuran (125 mL) n-butyl lithium (8.5 mL, 13.2 mmol) was added slowly at −78° C. and stirred for 1 hour. R-(−)-Glycidyl butyrate (1.7 mL, 12.1 mmol) was added at the same temperature and stirred over night at room temperature. Reaction mixture was quenched by the addition of saturated ammonium chloride solution. The organic layer was separated and dried over anhydrous sodium sulfate and volatiles were evaporated. The residue obtained was stirred in diethyl ether and the solid obtained was filtered to obtain a white solid.

Yield: 55%,

MS (m/z): 421 (M⁺−1),

¹H NMR (300 MHz, CDCl₃): δ 8.02 (t, J=8.5 Hz, 1H), 7.65-7.56 (m, 2H), 7.35 (dd, J=6.6 & 2.2 Hz, 1H), 4.86-4.74 (m, 3H), 4.13-3.98 (m, 3H), 3.84-3.74 (m, 1H), 2.11 (t, J=6.1 Hz, 1H), 0.95 (s, 9H), 0.15 (s, 6H).

### Preparation 34

### Tert-butyl 2-fluoro-4-(5-hydroxymethyl-2-oxo-oxazolidin-3-yl)-benzoate

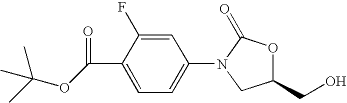

The title compound is prepared by following the procedure as described in preparation 33, by taking appropriate starting materials.

Yield: 71.3%,

¹H NMR (300 MHz, CDCl₃): δ 7.88 (t, J=8.7 Hz, 1H), 7.45 (dd, J=2.1 & 12.9 Hz, 1H), 7.28 (d, J=8.7 Hz, 1H), 4.83-4.72 (m, 1H), 4.92-3.96 (m, 4H), 3.84-3.72 (m, 2H), 2.28 (bs, 1H), 1.59 (s, 9H).

### Preparation 35

### 3-{4-[4-(Tert-butyl-dimethyl-silanyloxymethyl)-pyrazol-1-yl]-3-fluoro-phenyl}-5(R)-hydroxymethyl-oxazolidin-2-one

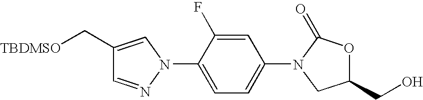

The title compound is prepared by following the procedure as described in preparation 33, by taking appropriate starting materials.

Yield: 69.2%,

MS (m/z): 422 (M⁺+1),

¹H NMR (300 MHz, CDCl₃): δ 7.90 (s, 1H), 7.86 (t, J=8.8 Hz, 1H), 7.75 (dd, J=2.5 & 13.9 Hz, 1H), 7.66 (s, 1H), 7.24 (dd, J=1.42 & 7.5 Hz, 1H), 4.75-4.85 (m, 1H), 4.70 (s, 2H), 3.99-4.10 (m, 3H), 3.72-3.82 (m, 1H), 2.23 (t, J=6.5 Hz, 1H), 0.93 (s, 9H), 0.18 (s, 6H).

### Preparation 36

### 3-{4-[4-(Tert-butyl-dimethyl-silanyloxymethyl)-imidazol-1-yl]-3-fluoro-phenyl}-5(R)-hydroxymethyl-oxazolidin-2-one

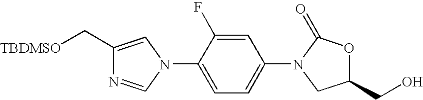

The title compound is prepared by following the procedure as described in preparation 33, by taking appropriate starting materials.

Yield: 58%,

¹H NMR (300 MHz, CDCl₃): δ 7.65-7.70 (m, 2H), 7.26-7.38 (m, 2H), 7.12 (s, 1H), 4.72-4.81 (m, 3H), 4.00-4.06 (m, 3H), 3.75-3.81 (m, 1H), 0.95 (s, 9H), 0.15 (s, 6H).

### Preparation 37

### Benzyl-(2-fluoro-4-(5-hydroxymethyl-2-oxo-oxazolidinyl-phenyl)-carbamate

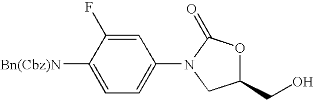

The title compound is prepared by following the procedure as described in preparation 33, by taking appropriate starting materials.

Yield: 50%,

IR (KBr, cm⁻¹): 3433, 3031, 1751, 1707, 1625, 1521, 1404, 1136, 1037, 753, 699,

MS (m/z): 451 (M⁺+1), 317,

¹H NMR (CDCl₃, 200 MHz): δ 7.51-6.95 (m, 13H), 5.15 (s, 2H), 4.80 (s, 2H), 4.73-4.64 (m, 1H), 4.17-3.72 (m, 5H).

### Preparation 38

### 3-(4-Amino-3-fluoro-phenyl)-5-hydroxymethyl-oxazolidin-2-one

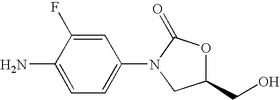

To a solution of benzyl-(2-fluoro-4-(5-hydroxymethyl-2-oxo-oxazolidinyl-phenyl)-carbamate (1.0 gram, 2.2 mmol) in methanol (15 mL) was added 10% palladium on charcoal (117.7 mg, 1.1 mmol). The reaction mixture was stirred under hydrogen atmosphere for 6-8 hours at room temperature. Palladium on charcoal was filtered off over a pad of celite and concentrated. The residue obtained was purified by column chromatography on silica gel using methanol and chloroform (1:19) as eluent to obtain the title compound.

Yield: 39%,

IR (KBr, cm⁻¹): 3414, 2926, 1729, 1729, 1524, 1425, 1232, 1184, 1038, 750,

MS (m/z): 226 (M⁺+1), 163, 152, 140,

¹H NMR (DMSO-d₆, 200 MHz): δ 7.37 (dd, J=2.4 & 13.7 Hz, 1H), 7.00-6.96 (m, 1H), 6.79-6.71 (m, 1H), 5.18 (t, J=5.4 Hz, 1H), 4.90 (bs, 2H), 4.75-4.62 (m, 1H), 3.98 (t, J=8.9 Hz, 1H), 3.76-3.47 (m, 2H).

### Preparation 39

### 3-(4-Azido-3-fluoro-phenyl)-5-hydroxymethyl-oxazolidin-2-one

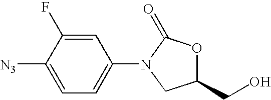

To a solution of 3-(4-amino-3-fluoro-phenyl)-5-hydroxymethyl-oxazolidin-2-one (200 mg, 0.89 mmol) in 50% aqueous hydrogen chloride (20 mL) was added sodium nitrite (122 mg, 1.77 mmol) in small batches at 5-10° C. Stirring was continued at same temperature for half an hour and then an aqueous solution of sodium azide (115.08 mg, 1.77 mmol) and sodium acetate (1.505 grams, 17.76 mmol) was added. Reaction mixture was diluted with water after 15 minutes and extracted with ethyl acetate (2×25 mL). Combined organic layer was washed with brine, dried over sodium sulfate and concentrated. The residue obtained was purified by column chromatography on silica gel using methanol and chloroform (1:19) as eluent to obtain the title compound.

Yield: 65%,

IR (KBr, cm⁻¹): 3457, 2933, 2852, 2126, 1724, 1511, 1428, 1319,

MS (m/z): 224 (M⁺-N₂), 149, 131,

¹H NMR (DMSO-d₆+CDCl₃, 400 MHz): δ 7.63 (dd, J=2.4 & 13.4 Hz, 1H); 7.23-7.10 (m, 1H); 7.20 (t, J=9.0 Hz, 1H); 5.03 (bs, 1H); 4.74-4.69 (m, 1H); 4.01-3.96 (m, 2H); 3.87 (dd, J=3.7 & 12.5 Hz, 1H); 3.70 (dd, J=3.7 & 12.5 Hz, 1H).

### Preparation 40

### 3-{4-[4-(Tert-butyl-dimethyl-silanyloxymethyl)-oxazol-2-yl]-3-fluoro-phenyl}-2-oxo-oxazolidin-5-ylmethyl methanesulfonate

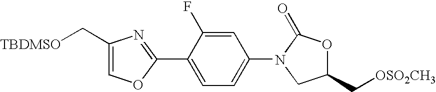

A solution of 3-{4-[4-(tert-butyl-dimethyl-silanyloxymethyl)-oxazol-2-yl]-3-fluoro-phenyl}-5-(R)-hydroxymethyl-oxazolidin-2-one (1.2 grams, 2.9 mmol) and diisopropylethylamine (2.25 mL, 14.3 mmol) in tetrahydrofuran (40 ml) was treated with methane sulfonyl chloride (0.7 mL, 8.1 mmol) at 0° C. for 10 minutes. Solvent was evaporated and the residue obtained was dissolved in ethyl acetate (50 mL). It was washed with water (2×25 mL), brine solution (25 mL) and dried over anhydrous sodium sulphate. The volatiles were evaporated to obtain the product as a pale yellow solid.

Yield: 98%,

¹H NMR (300 MHz, CDCl₃): δ 8.05 (t, J=8.4 Hz, 1H), 7.65 (s, 1H), 7.60 (dd, J=1.9 & 11.1 Hz, 1H), 7.34 (dd, J=2.1 & 6.7 Hz, 1H), 4.93-5.02 (m, 1H), 4.76 (s, 2H), 4.48 (dq, J=3.7 & 11.7 Hz, 2H), 4.20 (t, J=9.1 Hz, 1H), 4.00 (dd, J=2.9 & 6.2 Hz, 1H), 3.10 (s, 3H), 0.97 (s, 9H), 0.71 (s, 6H).

### Preparation 41

### 5-(R)-azidomethyl-3-{4-[4-(tert-butyl-dimethyl-silanyloxymethyl)-oxazol-2-yl]-3-fluoro-phenyl}-oxazolidin-2-one

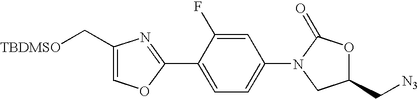

A solution of 3-{4-[4-(tert-butyl-dimethyl-silanyloxymethyl)-oxazol-2-yl]-3-fluoro-phenyl}-2-oxo-oxazolidin-5-ylmethyl methanesulfonate (3.0 grams, 6.0 mmol) in dimethylformamide (40 mL) was treated with sodium azide (1.17 grams, 18.0 mmol) at 75° C. for 2 hours. Solvent was evaporated and the residue formed was dissolved in water (20 mL). It was extracted with ethyl acetate (2×50 mL) and the organic layer was washed with brine solution. Finally it was dried over anhydrous sodium sulfate and the volatiles were evaporated to obtain a pale yellow solid.

Yield: 85%,

¹H NMR (300 MHz, CDCl₃): δ 8.03 (t, J=8.4 Hz, 1H), 7.63 (s, 1H), 7.58 (dd, J=2.1 & 11.0 Hz, 1H), 7.35 (dd, J=2.1 & 6.7 Hz, 1H), 4.87-4.37 (m, 1H), 4.75 (s, 2H), 4.12 (t, J=9.0 Hz, 1H), 3.90 (dd, J=2.6 & 6.3 Hz, 1H), 3.68 (dq, J=4.2 & 13.2 Hz, 2H), 0.93 (s, 9H), 0.15 (s, 6H).

### Preparation 42

### 5-(R)-azidomethyl-3-[3-fluoro-4-(4-hydroxymethyl-oxazol-2-yl)-phenyl]-oxazolidin-2-one

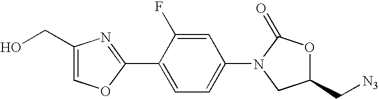

A solution of 5-(R)-azidomethyl-3-{4-[4-(tert-butyl-dimethyl-silanyloxymethyl)-oxazol-2-yl]-3-fluoro-phenyl}-oxazolidin-2-one (2.2 grams, 9.92 mmol) in tetrahydrofuran (4.4 mL) and water (4.4 mL) was treated with acetic acid (13.2 mL) at room temperature for 16 hours. The reaction mixture was neutralized by the addition of saturated sodium bicarbonate solution and extracted with ethyl acetate (2×50 mL). The organic layer was washed with brine solution and it was dried over anhydrous sodium sulfate. The volatiles were evaporated to obtain product as pale yellow solid.

Yield: 92%,

MS (m/z): 332 (M⁺−1),

### Preparation 43

### 5-(R)-azidomethyl-3-[4-(4-chloromethyl-oxazol-2-yl)-3-fluoro-phenyl]-oxazolidin-2-one

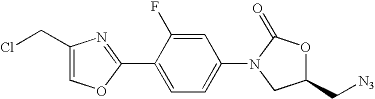

To a solution of 5-(R)-azidomethyl-3-[3-fluoro-4-(4-hydroxymethyl-oxazol-2-yl)-phenyl]-oxazolidin-2-one (1.5 grams, 4.50 mmol) in dichloromethane (7.5 mL), thionyl Chloride (7.5 mL) was added at 0° C. and stirred at room temperature for 30 minutes. Solvent was evaporated and the residue was dissolved in ethyl acetate (100 mL). Ethyl acetate solution was washed with water (2×50 mL), 10% sodium bicarbonate solution (50 mL) and brine solution (50 mL) successively. Finally it was dried over anhydrous sodium sulphate and volatiles were evaporated to obtain the product as a pale yellow solid.

Yield: 99%,

MS (m/z): 352 (M⁺+1),

¹H NMR: (400 MHz, CDCl₃): δ 8.05 (t, J=8.3 Hz, 1H), 7.75 (s, 1H), 7.60 (dt, J=2.1 & 11.0 Hz, 1H), 7.35 (dd, J=2.1 & 8.3 Hz, 1H), 4.88-4.79 (m, 1H), 4.60 (s, 2H), 4.18-4.08 (m, 1H), 3.93-3.86 (m, 1H), 3.68 (dq, J=4.2 & 13.2 Hz, 2H).

### Preparation 44

### Tert-butyl 4-(5-azidomethyl-2-oxo-oxazolidin-3-yl)-2-fluoro-benzoate

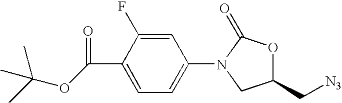

To a solution of tert-butyl 2-fluoro-4-(5-hydroxymethyl-2-oxo-oxazolidin-3-yl)-benzoate (30 grams, 96.37 mmol) and triethylamine (76.9 mL, 578.2 mmol) in dichloromethane was added methanesulphonyl chloride (28.2 mL, 289 mmol) dropwise at 0-5° C. The reaction mixture was allowed to come to room temperature and stirred for 3 hours. Dichloromethane was evaporated and the residue dissolved in ethyl acetate. Washing of the ethyl acetate portion with water and removal of ethyl acetate in a rotavapor left a mass (35 grams, 93%). To this mass in dimethylformamide (105 mL) was added sodium azide (17.5 grams, 169.6 mmol) and heated to 60° C. for 4 hours. Dimethylformamide was removed under reduced pressure and the resulting residue dissolved in ethyl acetate. Ethyl acetate portion was washed with water and brine successively and then concentrated to obtain the title compound.

Yield: 94%,

¹H NMR (300 MHz, CDCl₃): δ 7.89 (t, J=8.6 Hz, 1H), 7.47 (dd, J=2.4 & 12.9 Hz, 1H), 7.27 (dd, J=2.4 & 9.3 Hz, 1H), 4.83-4.80 (m, 1H), 4.10 (t, J=9.0 Hz, 1H), 3.87 (dd, J=6.3 & 9.0 Hz, 1H), 3.57-3.78 (m, 2H), 1.59 (s, 9H).

### Preparation 45

### 4-(5-Azidomethyl-2-oxo-oxazolidin-3-yl)-2-fluoro-benzoic acid

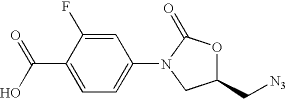

Tert-Butyl 4-(5-azidomethyl-2-oxo-oxazolidin-3-yl)-2-fluoro-benzoate (28 grams, 83.33 mmol) was dissolved in dichloromethane (110 mL). Trifluoroacetic acid (110 mL) was added to it drop wise at 0-5° C. and stirred at room temperature for 2 hours. Trifluoroacetic acid and dichloromethane were completely evaporated. Hexane was added to the residue obtained and stirred for a while. The solid obtained was filtered and dried.

Yield: 90.5%,

¹H NMR (300 MHz, CD₃OD): δ 8.01 (t, J=8.6 Hz, 1H), 7.66 (dd, J=2.1 & 13.5 Hz, 1H), 4.95 (m, 1H), 4.23 (t, J=9.2 Hz, 1H), 3.94 (dd, J=6.2 & 9.3 Hz, 1H), 3.82-3.64 (m, 2H).

### Preparation 46

### 4-(5-Aminomethyl-2-oxo-oxazolidin-3-yl)-2-fluoro-benzamide

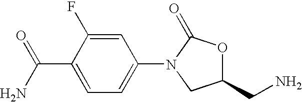

To a tetrahydrofuran solution of 4-(5-azidomethyl-2-oxo-oxazolidin-3-yl)-2-fluoro-benzamide (17 grams, 60.9 mmol) was added triphenyl phosphene (20.8 grams, 79.1 mmol) at room temperature and stirred for 2 hours. Water (12 mL) was added to the reaction mixture and refluxed at 90° C. for 5 hours. The reaction mixture was then concentrated and the residue triturated with benzene. Benzene was decanted and the trituration process was repeated for 4-5 times. Solid obtained by this way was dried and used for the next step.

Yield: 73%.

¹H NMR (300 MHz, CDCl₃): δ 7.75 (t, J=8.63 Hz, 1H), 7.61-7.53 (m, 3H), 7.41 (dd, J=2.1 & 8.7 Hz, 1H), 4.72-4.62 (m, 1H), 4.10 (t, J=9.0 Hz, 1H), 3.89 (dd, J=6.4 & 9.0 Hz, 1H), 2.94-2.78 (m, 2H).

### Preparation 47

### 4-(5-Azidomethyl-2-oxo-oxazolidin-3-yl)-2-fluoro-benzamide

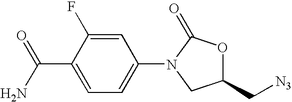

To a solution of 4-(5-azidomethyl-2-oxo-oxazolidin-3-yl)-2-fluoro-benzoic acid (12.5 grams, 44.6 mmol) in dichloromethane was added thionyl chloride (62.5 mL) and heated to 60° C. for 3 hours. Thionyl chloride was removed completely and ammonia solution (100 mL) was added to the resulting residue at 0° C. Temperature was allowed to come to room temperature and stirred for 2 hours. Chloroform was added and washed with brine solution. Chloroform layer was dried over sodium sulfate and concentrated to obtain the title compound.

Yield: 96%,

¹H NMR (300 MHz, DMSO-d₆): δ 7.75 (t, J=8.4 Hz, 1H), 7.62-7.52 (m, 3H), 7.40 (dd, J=2.1 & 8.7 Hz, 1H), 4.98-4.88 (m, 1H), 4.17 (t, J=9.3 Hz, 1H), 3.86-3.66 (m, 3H).

### Preparation 48

### (R)-4-(5-(Azidomethyl)-2-oxooxazolidin-3-yl)-2-fluoro-M-(2-(pyridin-2-yl)acetyl)benzohydrazide

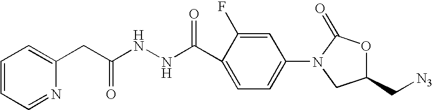

The title compound is prepared by following the procedure as described in preparation 47, by taking appropriate starting materials.

Yield: 59%

¹HNMR (400 MHz, DMSO): δ 10.20 (bs, 2H), 8.50-8.47 (m, 1H), 7.80-7.65 (m, 2H), 7.55 (dd, J=1.9 Hz, 7.8 Hz, 1H), 7.46-7.38 (m, 2H), 7.28-7.24 (m, 1H), 4.95-4.85 (m, 1H), 4.18 (t, J=9.4 Hz, 1H), 3.85-3.78 (m, 1H), 3.72 (s, 2H), 3.35-3.25 (m, 2H).

ES-MS (m/z): 412.5 (M⁺)

### Preparation 49

### 5-(R)-azidomethyl-3-{4-[4-(tert-butyl-dimethyl-silanyloxymethyl)-pyrazol-1-yl]-3-fluoro-phenyl}-oxazolidin-2-one

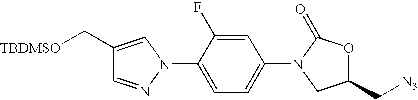

To a solution of 3-{4-[4-(tert-butyl-dimethyl-silanyloxymethyl)-pyrazol-1-yl]-3-fluoro-phenyl}-2-oxo-oxazolidin-5-(R)-ylmethyl methanesulfonate (7.46 grams, 14.95 mmol) in dimethylformamide (100 mL) was added sodium azide (4.86 grams, 74.73 mmol) and stirred at 75° C. for 2.5 hours. Solvent was removed by distillation under reduced pressure. Water (100 mL) was added to the residue and extracted with ethyl acetate (150 mL×2). Combined ethyl acetate layer was washed with brine (100 mL×1) and concentrated to obtain 4.83 grams of title compound as solid.

Yield: 72.55%,

MS (m/z): 447 (M⁺+1),

¹H NMR (300 MHz, CDCl₃): δ 7.85-7.92 (m, 2H), 7.67-7.80 (m, 2H), 7.22 (dd, J=1.6 & 8.9 Hz, 1H), 4.80-4.87 (m, 1H), 4.71 (s, 2H), 4.12 (t, J=9.0 Hz, 1H), 3.90 (dd, J=2.6 & 6.3 Hz, 1H), 3.68 (dq, J=4.4 & 13.3 Hz, 2H), 0.93 (s, 9H), 0.20 (s, 6H).

### Preparation 50

### 5-(R)-azidomethyl-3-[3-fluoro-4-(4-hydroxymethyl-pyrazol-1-yl)-phenyl]-oxazolidin-2-one

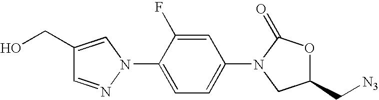

To a mixture of tetrahydrofuran (5 mL), water (5 mL) and acetic acid (15 mL) was added 5-(R)-azidomethyl-3-{-4-[4-(tert-butyl-dimethyl-silanyloxymethyl)-pyrazol-1-yl]-3-fluoro-phenyl}-oxazolidin-2-one (4.83 grams, 14.5 mmol) and stirred overnight at room temperature 10% sodium bicarbonate solution was added to the reaction mixture and diluted with ethyl acetate. Organic layer was separated and washed with 5% ammonia solution and brine successively. Finally it was dried over anhydrous sodium sulfate and concentrated to obtain 3.59 grams of title compound as solid.

Yield: 100%,

MS (m/z): 333 (M⁺+1),

¹H NMR (300 MHz, CDCl₃): δ 8.00 (d, J=2.7 Hz, 1H), 7.89 (t, J=8.9 Hz, 1H), 7.72-7.79 (m, 2H), 7.22 (dd, J=1.5 & 7.5 Hz, 1H), 4.81-4.88 (m, 1H), 4.71 (s, 2H), 4.18 (t, J=8.9 Hz, 1H), 3.91 (dd, J=2.6 & 6.2 Hz, 1H), 3.69 (dq, J=4.3 & 13.3 Hz, 2H), 1.71 (bs, 1H).

### Preparation 51

### 5-Azidomethyl-3-[3-chloromethyl-imidazol-1-yl)-3-fluoro-phenyl]-oxazolidin-2-one

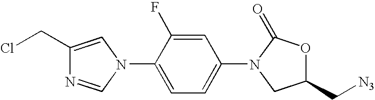

A mixture of 5-azidomethyl-3-[3-fluoro-4-(hydroxymethyl-imidazol-1-yl)-phenyl]-oxazolidin-2-one (7 grams, 21 mmol) and thionyl chloride (175 mL) was stirred at room temperature for 16 hours. Thionyl chloride was evaporated and the residue was treated with aqueous sodiumbicarbonate solution at 5° C. to neutral pH. It was extracted with ethyl acetate (100 mL×2) and the combined organic layer was washed with brine (100 mL). Finally it was dried over anhydrous sodium sulfate and volatiles were evaporated to obtain the title compound.

Yield: 96%,

¹H NMR (300 MHz, CDCl₃): δ 8.0 (s, 1H), 7.69-7.80 (m, 2H), 7.32-7.44 (m, 2H), 4.82-4.95 (m, 1H), 4.61 (s, 2H), 4.12 (t, J=8.9 Hz, 1H), 3.88-3.94 (m, 1H), 3.72-3.82 (m, 1H), 3.65 (dd, J=5.1 & 13.5 Hz, 1H).

### Preparation 52

### 5-(R)-azidomethyl-3-[4-(4-chloromethyl-pyrazol-1-yl)-3-fluoro-phenyl]-oxazolidin-2-one

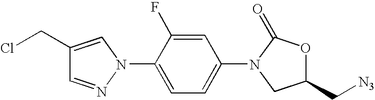

The title compound is prepared by following the procedure as described in preparation 51, by taking appropriate starting materials.

Yield: 99%,

MS (m/z): 352 (M⁺+1),

¹H NMR (300 MHz, CDCl₃) δ 8.03 (d, J=2.4 Hz, 1H), 7.90 (t, J=8.9 Hz, 1H), 7.73-7.82 (m, 2H), 7.24 (dd, J=1.4 & 10.9 Hz, 1H), 4.79-4.89 (m, 1H), 4.64 (s, 2H), 4.12 (t, J=8.9 Hz, 1H), 3.91 (dd, J=2.6 & 6.2 Hz, 1H), 3.69 (dq, J=4.5 & 13.3 Hz, 2H).

### Preparation 53

### 5-Azidomethyl-3-{4-[4-(tert-butyl-dimethyl-silanyloxymethyl)-imidazol-1-yl]-3-fluoro-phenyl}-oxazolidin-2-one

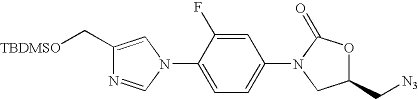

The title compound is prepared by following the procedure as described in preparation 49, by taking appropriate starting materials.

Yield: 98%,

¹H NMR (300 MHz, CDCl₃): δ 7.68-7.75 (m, 2H), 7.29-7.44 (m, 2H), 7.16 (s, 1H), 4.79-4.88 (m, 1H), 4.80 (s, 2H), 4.10-4.18 (m, 2H), 3.65 (dd, J=4.2 & 13.0 Hz, 1H), 0.97 (s, 9H), 0.12 (s, 6H).

### Preparation 54

### 5-Azidomethyl-3-[3-fluoro-4-(hydroxymethyl-imidazol-1-yl)-phenyl]-oxazolidin-2-one

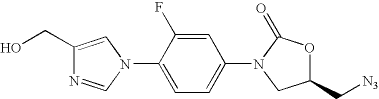

A solution of 5-azidomethyl-3-{4-[4-(tert-butyl-dimethyl-silanyloxymethyl)-imidazol-1-yl]-3-fluoro-phenyl}-oxazolidin-2-one (35 grams, 447 mmol) in a mixture of acetic acid:trifluoroacetic acid:water (3:1:1, 875 mL) was stirred at room temperature for 16 hours. Solvents were evaporated under rotary evaporation and the residue obtained was scratched in ether to obtain white solid. It was filtered and dried under vacuum.

Yield: 91%,

¹H NMR (300 MHz, CD₃OD): δ 7.96 (t, J=1.2 Hz, 1H), 7.84 (dd, J=2.4 & 13.5 Hz, 1H), 7.61 (m, 1H), 7.48-7.52 (m, 1H), 4.80-4.90 (m, 1H), 7.41 (s, 1H), 4.60 (s, 2H), 4.24 (t, J=9.0 Hz, 1H), 3.92-3.97 (m, 1H), 3.78-3.84 (m, 1H), 3.65 (dd, J=5.1 & 13.5 Hz, 1H).

### Preparation 55

### Benzyl-(4-[5-azidomethyl-2-oxo-oxazolidin-3-yl)-2-fluoro-phenyl]-carbamate

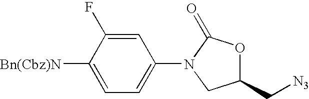

The title compound is prepared by following the procedure as described in preparation 44, by taking appropriate starting materials.

Yield: 65%,

IR (KBr, cm⁻¹): 3386, 3032, 2928, 2108, 1707, 1674, 1521, 1403, 1297, 1218, 1133, 1036, 752, 699,

MS (m/z): 476 (M⁺+1), 420, 419, 384,

¹H NMR (CDCl₃, 200 MHz): δ 7.51-6.97 (m, 13H), 5.16 (s, 2H), 4.81 (s, 2H), 4.75-4.60 (m, 1H), 4.01-3.15 (m, 4H).

### Preparation 56

### Tert-butyl [3-(4-carbamoyl-3-fluoro-phenyl)-2-oxo-oxazolidin-5(S)-ylmethyl]-carbamate

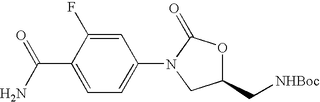

To a solution of 4-(5(S)-aminomethyl-2-oxo-oxazolidin-3-yl)-2-fluoro-benzamide (8 grams, 31.6 mmol) and triethylamine (13.2 mL, 94.8 mmol) in tetrahydrofuran/methanol (1:1) mixture was added Di-tert-Butyl dicarbonate (10.8 mL, 47.4 mmol) drop wise at 0-5° C. Reaction mixture was then stirred at room temperature overnight. Solvent was evaporated and the residue dissolved in ethyl acetate. Ethyl acetate layer was washed with water 3-4 times, dried over sodium sulfate and concentrated to obtain the title compound.

Yield: 90%,

¹H NMR (300 MHz, CDCl₃): δ 8.01 (t, J=8.8 Hz, 1H), 7.63 (dd, J=2.0 & 14.8 Hz, 1H), 7.1 (d, J=8.4 Hz, 1H), 5.52 (bs, 2H), 5.01 (bs, 1H), 4.65-4.75 (m, 1H), 4.01 (t, J=8.9 Hz, 1H), 3.82 (dd, J=6.5 & 8.9 Hz, 1H), 3.40-3.32 (m, 2H), 1.32 (s, 9H).

### Preparation 57

### Preparation of tert-butyl [3-(3-fluoro-4-thiocarbamoyl-phenyl)-2-oxo-oxazolidin-5(S)-ylmethyl]-carbamate

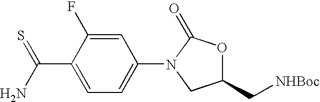

To a solution of tert-butyl [3-(4-carbamoyl-3-fluoro-phenyl)-2-oxo-oxazolidin-5(S)-ylmethyl]-carbamate (11 grams, 31.1 mmol) in dry dioxane was added Lawesson's reagent (7.56 grams, 18.7 mmol) and heated to 60° C. for 1 hour. Dioxane was evaporated completely and the residue dissolved in ethyl acetate. The ethyl acetate layer was washed with water 8-10 times, dried over sodium sulfate and concentrated. Pure product (8.2 grams) was obtained by column chromatographic purification on basic alumina using 1% methanol in chloroform as eluent.

Yield: 71.3%,

¹H NMR (300 MHz, CDCl₃): δ 8.49 (t, J=9.3 Hz, 1H), 7.86 (bs, 2H), 7.72 (dd, J=2.2 & 15.5 Hz, 1H), 7.12 (dd, J=1.8 & 8.8 Hz, 1H), 4.97 (bs, 1H), 4.83-4.78 (m, 1H), 4.08 (t, J=9.1 Hz, 1H), 3.90 (dd, J=6.7 & 8.9 Hz, 1H), 3.56 (dd, J=4.2 & 6.4 Hz, 2H), 1.42 (s, 9H).

### Preparation 58

### Tert-butyl {3-[4-(4-chloromethyl-thiazol-2-yl)-3-fluoro-phenyl]-2-oxo-oxazolidin-5(S)-ylmethyl}-carbamate

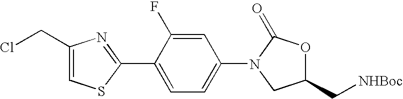

A mixture of tert-butyl [3-(3-fluoro-4-thiocarbamoyl-phenyl)-2-oxo-oxazolidin-5(S)-ylmethyl]-carbamate (5 grams, 13.5 mmol) and dichloroacetone (1.7 grams, 13.5 mmol) in toluene was refluxed at 110° C. for 2 hours. Toluene was evaporated and the residue purified by column chromatography over basic alumina using 1% methanol in chloroform to obtain pure product.

Yield: 47%,

¹H NMR (300 MHz, CDCl₃): δ 7.90 (t, J=8.7 Hz, 1H), 7.41 (dd, J=2.2 & 13.5, 1H), 7.21 (bs, 1H), 5.01 (bs, 1H), 4.79 (bs, 1H), 4.71 (s, 2H), 4.10 (bs, 1H), 3.90 (bs, 1H), 3.50 (bs, 2H), 1.41 (s, 9H).

### Preparation 59

### 3-{4-[4-(Tert-butyl-dimethyl-silanyloxymethyl)-pyrazol-1-yl]-3-fluoro-phenyl}-2-oxo-oxazolidin-5-(R)-ylmethyl methanesulfonate

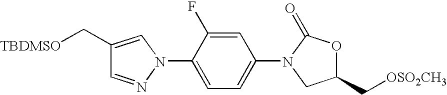

To a solution of 3-{4-[4-(tert-butyl-dimethyl-silanyloxymethyl)-pyrazol-1-yl]-3,5-difluoro-phenyl}-5-(R)-hydroxymethyl-oxazolidin-2-one (5.5 grams, 13.06 mmol) in dichloromethane (50 mL) was added triethylamine (7.27 mL, 52.26 mmol) and cooled to 0° C. Methane sulfonylchloride (2.02 mL, 26.13 mmol) was added to it and stirred for 40 minutes at room temperature. Reaction mixture was then diluted with dichloromethane and washed with water (200 mL×2) and brine solution (200 mL×1) successively. Organic layer was dried over anhydrous sodium sulfate and concentrated to obtain 6.5 grams of white solid.

Yield: 99.8%,

MS (m/z): 500 (M⁺+1),

¹H NMR (300 MHz, CDCl₃): δ 7.85-8.00 (m, 2H), 7.67-7.74 (m, 2H), 7.21-7.26 (m, 1H), 4.91-5.02 (m, 1H), 4.70 (d, J=7.3 Hz, 2H), 4.49 (dq, J=3.5 & 11.7 Hz, 2H), 4.19 (t, J=9.1 Hz, 1H), 3.99 (t, J=6.4 Hz, 1H), 3.12 (s, 3H), 0.95 (s, 9H), 0.20 (s, 6H).

### Preparation 60

### 3-{4-[4-(Tert-butyl-dimethyl-silanyloxymethyl)-imidazol-1-yl]-3-fluoro-phenyl}-2-oxo-oxazolidin-5-ylmethyl methanesulfonate

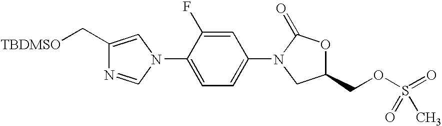

The title compound is prepared by following the procedure as described in preparation 59, by taking appropriate starting materials.

Yield: 95.2%,

¹H NMR (300 MHz, CDCl₃): δ 7.65-7.72 (m, 2H), 7.25-7.50 (m, 2H), 7.10 (s, 1H), 4.96-4.97 (m, 1H), 4.75 (d, J=0.9 Hz, 2H), 4.42-4.51 (m, 2H), 4.07-4.21 (m, 2H), 3.10 (s, 3H), 0.93 (s, 9H), 0.12 (s, 6H).

### Preparation 61

### (S)-4-(5-(aminomethyl)-2-oxooxazolidin-3-yl)-2-fluoro-N′-(2-(pyridin-2-yl)acetyl)benzohydrazide

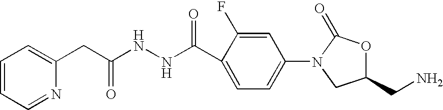

To a solution of (R)-4-(5-(azidomethyl)-2-oxooxazolidin-3-yl)-2-fluoro-N′-(2-(pyridin-2-yl)acetyl)benzohydrazide (2 grams) in methanol-dioxane mixture, 20% Pd—C was added and the reaction mixture was hydrogenated (50 Psi) for 6 hours. The solution was finally filtered over celite bed; the filterate was concentrated under reduced pressure. The crude residue (1.4 grams) was taken for the next step.

### Preparation 62

### (S)-tert-butyl (3-(3-fluoro-4-(2-(2-(pyridin-2-yl)acetyl)hydrazinecarbonyl)phenyl)-2-oxooxazolidin-5-yl)methylcarbamate

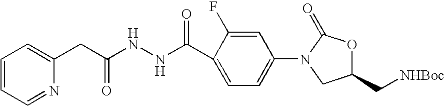

The crude (S)-4-(5-(aminomethyl)-2-oxooxazolidin-3-yl)-2-fluoro-N′-(2-(pyridin-2-yl)acetyl)benzohydrazide (1.4 grams, 3.62 mmol) was dissolved in THF-H₂O (2:1) mixture and sodium bicarbonate (900 mg, 10.71 mmol) was added to it. Then, (Boc)₂O (1.18 grams, 5.41 mmol) was added dropwise to it at 0° C. and the reaction mixture was stirred at room temperature for overnight. Finally it was diluted with water and extracted with ethyl acetate. The solvent was removed under reduced pressure and the residue was purified by column chromatography 1 gram.

Yield: 57%.

IR (KBr, cm⁻¹): 3298, 2976, 1749, 1678, 1624, 1516, 1410, 1167, 1001, 918, 872, 752

¹HNMR (400 MHz, DMSO): δ 10.27 (s, 1H), 10.13 (s, 1H), 8.49-8.48 (m, 1H), 7.77 (dd, J=1.6 Hz, 7.5 Hz, 1H)), 7.68 (t, J=8.3 Hz, 1H), 7.57 (dd, J=1.9 Hz, 13.2 Hz, 1H), 7.46-7.38 (m, 2H), 7.34-7.26 (m, 1H), 7.22-7.18 (m, 1H), 4.80-4.70 (m, 1H), 4.14 (t, J=9.1 Hz, 1H), 3.86-3.80 (m, 1H), 3.73 (s, 2H), 3.32-3.24 (m, 2H), 1.35 (s, 9H).

ES-MS (m/z): 488 (M⁺+1)

### Preparation 63

### 1-Prop-2-yne-1H-pyrazol

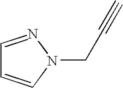

A solution of pyrazole (2.0 grams, 29.4 mmol) and dry potassium carbonate (6.1 grams, 44.1 mmol) in dimethylformamide (20 mL) was treated with propargyl bromide (5.75 grams, 44.1 mmol) for 16 hours at room temperature. Inorganic material was filtered off and filtrate was diluted with water (100 mL). It was then extracted with ethyl acetate (2×100 mL) and the organic layer was washed with brine. Finally it was dried over sodium sulfate and volatiles were evaporated. The residue obtained was purified by column chromatography on silica gel using ethyl acetate and petroleum ether (1:4) to obtain the title compound as brown liquid.

Yield: 55%,

IR (Neat, cm⁻¹): 3290, 3060, 2926, 2126, 1599, 1396, 1089, 1050,

MS (m/z): 107 (M⁺+1), 99,

¹H NMR (CDCl₃, 200 MHz): δ 7.59-7.30 (m, 2H), 6.23 (s, 1H), 4.93 (s, 2H), 2.50 (s, 1H).

### Preparation 64

### Ethyl 1-prop-2-yne 3-amino-1H-pyrazol-4-caraboxylate

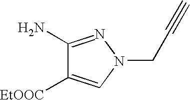

The title compound is prepared by following the procedure as described in preparation 63, by taking appropriate starting materials.

Yield: 12%,

IR (Neat, cm⁻¹): 3442, 3294, 2981, 1682, 1552, 1384, 1231, 1100,

MS (m/z): 194 (M⁺+1), 99,

¹H NMR (CDCl₃, 200 MHz): δ 7.61 (s, 1H), 5.23 (bs, 2H), 4.77 (d, J=2.5 Hz, 2H), 4.30 (q, J=7.1 Hz, 2H), 2.50 (t, J=2.5 Hz, 1H), 1.34 (t, J=7.1 Hz, 3H).

### Preparation 65

### 1-Prop-2-ynyl-1H-[1,2,4]triazole

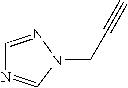

The title compound is prepared by following the procedure as described in preparation 63, by taking appropriate starting materials.

Yield: 89%,

IR (Neat, cm⁻¹): 3124, 2970, 2129, 1505, 1427, 1275, 1138, 1018,

MS (m/z): 105 (M⁺−1), 97, 91,

¹H NMR (CDCl₃, 200 MHz): δ 8.31 (s, 1H), 7.97 (s, 1H), 5.00 (d, J=2.7 Hz, 2H), 2.63 (t, J=2.5 Hz, 1H).

### Preparation 66

### 3-(Trifluoromethyl)-1-prop-2-ynyl-1H-pyrazole

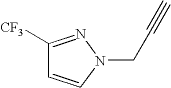

The title compound is prepared by following the procedure as described in preparation 63, by taking appropriate starting materials.

Yield: 40%,

IR (Neat, cm⁻¹): 3309, 2927, 1742, 1384, 1242, 1135, 968, 771, 675,

MS (m/z): 175 (M⁺+1), 149,

¹H NMR (CDCl₃, 400 MHz): δ 7.70 (d, J=1.3 Hz, 1H), 6.57 (d, J=2.4 Hz, 1H), 4.99 (d, J=2.4 Hz, 2H), 2.57 (t, J=2.6 Hz, 1H).

### Preparation 67

### 3-(1H-Imidazol-4-yl)-pyridine

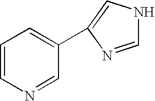

This compound was synthesized as per the procedure mentioned in J. Med. Chem., 1998, 41, 2404. The crude compound (7 g) obtained was directly taken for further step.

### Preparation 68

### 3-(1-Prop-2-ynyl-1H-imidazol-4-yl)-pyridine

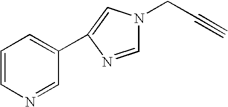

To a solution of 3-(1H-Imidazol-4-yl)-pyridine (750 mg, 5.17 mmol) in THF was added NaH (310 mg, 7.76 mmol) followed by the addition of propargyl bromide (1.35 mL, 15.51 mmol) at 0° C. and the resulting mixture was stirred at room temperature for 3 hours. The reaction mixture was first quenched with water and then extracted with ethyl acetate, washed with water and brine. The combined organic layers were dried over Na₂SO₄ and the concentrated under vacuum yield crude product (450 mg) that was directly taken for further step.

Yield: 47%

ES-MS (m/z): 184 (M⁺+1)

### Preparation 69

### 3-Methyl-4-prop-2-ynyl-4H-[1,2,4]triazole

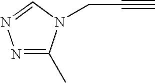

The above compound was synthesized in 11% yield following the procedure mentioned in Org. Lett., 2004, 6, 17, 2969

### Preparation 70

### 1-(Pyridin-2-yl)prop-2-yn-1-ol

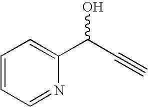

To a solution of pyridin-2-carboxaldehyde (2 grams, 18.69 mmol) in THF, ethynylmagnesium bromide (45 mL, 0.5M in THF, 22.43 mmol) was added at 0° C., and the reaction mixture was stirred at room temperature for 3 hours. The reaction was quenched with saturated aqueous ammonium chloride and extracted with ethyl acetate. The organic layer was concentrated under reduced pressure and the crude residue was used for the next step. Following the similar procedure 1-(pyridin-3-yl)prop-2-yn-1-ol and 1-(pyridin-4-yl)prop-2-yn-1-ol were synthesized.

### Preparation 71

### Benzyl-{2-fluoro-4-[5-(methoxycarbonylamino-methyl)-2-oxo-oxazolidin-3-yl]-phenyl}-carbamate

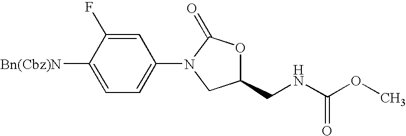

To a solution of benzyl-[5-aminomethyl-2-oxo-oxazolidin-3-yl)-2-fluoro-phenyl]-carbamate (1 gram, 2.22 mmol) and triethylamine (337 mg, 3.33 mmol) in chloroform (25 mL) was added chloromethylformate (252 mg, 2.66 mmol) at 0° C. The reaction mixture was allowed to come to room temperature and stirred for additional half an hour. Water (200 mL) was added to the reaction mixture and extracted with chloroform (2×100 mL). Organic layer was separated and washed with brine. Finally it was dried over sodium sulfate and volatiles were evaporated. The residue obtained was purified by column chromatography on silica gel using methanol and chloroform (1:9) as eluent to obtain title compound as white solid.

Yield: 70%,

IR (KBr, cm⁻¹): 3356, 2926, 1756, 1709, 1520, 1043, 1218,

MS (m/z): 548, 508 (M⁺+1), 476, 444, 412, 374, 310, 279,

¹H NMR (CDCl₃, 200 MHz): δ 7.50-7.04 (m, 13H), 5.16-5.0 (m, 2H), 4.98-4.60 (m, 3H), 4.97 (t, J=8.4 Hz, 1H), 3.98-3.40 (m, 7H).

### Preparation 72

### Methyl [3-(4-Amino-3-fluoro-phenyl)-2-oxo-oxazolidin-5-yl-methyl]-carbamate

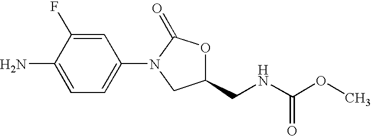

A solution of benzyl-{2-fluoro-4-[5-(methoxycarbonylamino-methyl)-2-oxo-oxazolidin-3-yl]-phenyl}-carbamate (2 grams, 4.0 mmol) in methanol (25 mL) was stirred under hydrogen atmosphere for 8 hours in the presence of 10% palladium on charcoal (637 mg, 0.6 mmol). Catalyst was filtered over a pad of celite and the filtrate was concentrated. The residue obtained was purified by column chromatography on silica gel using methanol and chloroform (1:19) as eluent to get title compound as white solid.

Yield: 60%,

IR (KBr, cm⁻¹): 3398, 2926, 1722, 1632, 1522, 1428, 1365,

MS (m/z): 284 (M⁺), 266, 252,

¹H NMR (DMSO-d₆, 400 MHz): δ 7.64-7.48 (m, 2H), 7.38 (dd, J=2.4 & 13.4 Hz, 1H), 6.96 (dd, J=2.2 & 8.6 Hz, 1H), 6.76 (dd, J=8.6 & 9.9 Hz, 1H), 5.0 (s, 2H), 4.70-4.62 (m, 1H), 4.01 (t, J=9.1 Hz, 1H), 3.64 (dd, J=6.1 & 9.1 Hz, 1H), 3.54 (s, 3H), 3.33 (t, J=6.1 Hz, 2H).

### Preparation 73

### Methyl [3-(4-azido-3-fluoro-phenyl)-2-oxo-oxazolidin-5-yl-methyl]-carbamate

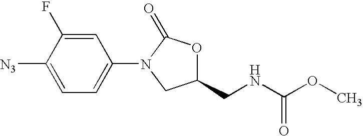

To a solution of methyl [3-(4-amino-3-fluoro-phenyl)-2-oxo-oxazolidin-5-yl-methyl]-carbamate (225 mg, 0.795 mmol) in 50% aqueous hydrochloric acid (25 mL) was added sodium nitirite (109 mg, 0.16 mmol) in small batches at 5-10° C. and stirred for half an hour. A solution of sodium azide (104 mg, 0.16 mmol) and sodium acetate (1.37 grams, 16 mmol) was added to the reaction mixture over 15 minutes. The reaction mixture was then diluted with water and extracted with ethyl acetate (2×50 mL). The organic layer was washed with brine and dried over sodium sulfate. The volatiles were evaporated and the residue obtained was purified by column chromatography on silica gel using methanol and chloroform (1:9) to get the title compound as brown solid.

Yield: 57%,

IR (KBr, cm⁻¹): 3356, 2922, 2852, 2136, 2103, 1730, 1600, 1426, 1324,

MS (m/z): 310 (M⁺), 284, 269, 251,

¹H NMR (DMSO-d₆, 400 MHz): δ 7.64-7.50 (m, 2H), 7.36-7.30 (m, 2H), 4.75-4.70 (m, 1H), 4.11 (t, J=8.8 Hz, 1H), 3.76 (dd, J=6.4 & 9.1 Hz, 1H), 3.54 (s, 3H), 3.35 (t, J=5.9 Hz, 2H).

### Preparation 74

### 3-[3-Fluoro-4-(4-pyrazol-1-ylmethyl-imidazol-1-yl)-phenyl]-5-isothiocyanatomethyl-oxazolidin-2-one

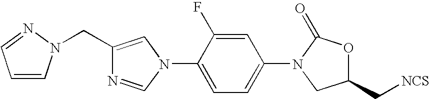

To a solution of 5-aminomethyl-3-[3-fluoro-4-(4-pyrazol-1-ylmethyl-imidazol-1-yl)-phenyl]-oxazolidin-2-one (0.6 grams, 1.7 mmol) in dichloromethane (15 mL) was added triethyl amine (0.7 mL, 4.95 mmol) followed by the drop wise addition of thiophosgene (0.15 mL, 1.98 mmol) at 0-5° C. The resulting mixture was stirred at the same temperature for 30 minutes. Solvent was evaporated and the residue obtained was purified by column chromatography on silica gel (100-200 mesh) using a mixture of methanol and chloroform (1:19) to obtain the title compound.

Yield: 27%,

### Preparation 75

### 5-Aminomethyl-3-{4-[4-(tert-butyl-dimethyl-silanyloxymethyl)-imidazol-1-yl]-3-fluoro-phenyl}-oxazolidin-2-one

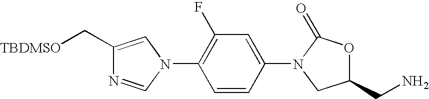

To a solution of 5-Azidomethyl-3-{4-[4-(tert-butyl-dimethyl-silanyloxymethyl)-imidazol-1-yl]-3-fluoro-phenyl}-oxazolidin-2-one (13.0 grams, 29.15 mmol) in THF was added triphenyl phosphine (8.6 grams, 38.05 mmol) followed by the addition of 1 mL of water after 1 hours. The reaction mixture was heated at 50° C. for overnight. The solvent was evaporated under reduced pressure and the residue was purified by column chromatography (7.20 grams)

Yield: 59%

¹HNMR (400 MHz, DMSO): δ 7.92 (t, J=1.3 Hz, 1H), 7.74 (dd, J=2.4, 13.7 Hz, 1H), 7.65 (t, J=8.9 Hz, 1H), 7.47 (dd, J=1.1, 8.9 Hz, 1H), 7.36 (s, 1H), 4.67-4.65 (m, 1H), 4.61 (s, 2H), 4.12 (t, J=9.1 Hz, 1H), 3.91 (dd, J=6.4, 9.1 Hz, 1H), 3.25 (bs, 2H), 2.85 (dd, J=4.8, 12.3 Hz, 2H), 0.89 (s, 9H), 0.08 (s, 6H)

ES-MS (m/z): 421 (M⁺+1)

### Preparation 76

### (S)-N-(3-{4-[4-(tert-Butyl-dimethyl-silanyloxymethyl)-imidazol-1-yl]-3-fluoro-phenyl}-oxazolidin-5-ylmethyl)-acetamide

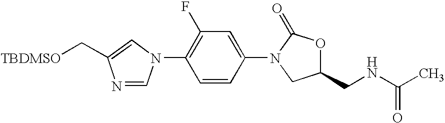

To a solution of 5-Aminomethyl-3-{4-[4-(tert-butyl-dimethyl-silanyloxymethyl)-imidazol-1-yl]-3-fluoro-phenyl}-oxazolidin-2-one (7.0 grams, 16.6 mmol) in DCM was added triethylamine (6.95 mL, 50.0 mmol) followed by the addition of acetic anhydride (4.75 mL, 49.9 mmol) at 0° C. and the resulting mixture was stirred room temperature for 12 hours. The reaction mixture was extracted with DCM, washed with water and brine. Organic layer was dried over Na₂SO₄ and concentrated under vacuum. The crude product was purified by column chromatography to obtain the desired product as a white solid (6.20 grams)

Yield: 80%

IR (Neat, cm⁻¹): 3275, 1755, 1662, 1529, 1416, 1223, 839, 752

¹H NMR: (400 MHz, DMSO): δ 8.24 (t, J=5.9 Hz, 1H), 7.94 (s, 1H), 7.72 (dd, J=2.3, 13.4 Hz, 1H), 7.66 (t, J=8.9 Hz, 1H), 7.44 (dd, J=1.9, 8.9 Hz, 1H), 7.37 (s, 1H), 4.81-4.74 (m, 1H), 4.61 (s, 2H), 4.17 (t, J=8.9 Hz, 1H), 3.78 (dd, J=6.4, 9.1 Hz, 1H), 3.44 (t, J=5.5 Hz, 2H), 1.84 (s, 3H), 0.89 (s, 9H), 0.08 (s, 6H).

ES-MS (m/z): 463 (M⁺+1)

### Preparation 77

### (S)-N-{3-[3-Fluoro-4-(4-hydroxymethyl-imidazol-1-yl)-phenyl]-2-oxo-oxazolidin-5-ylmethyl}-acetamide

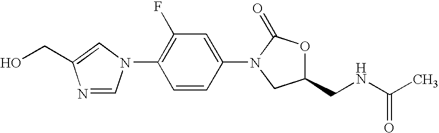

To a solution of 2-oxo-N-(3-{4-[4-(tert-Butyl-dimethyl-silanyloxymethyl)-imidazol-1-yl]-3-fluoro-phenyl}-oxazolidin-5-ylmethyl)-acetamide (6.20 grams, 13.4 mmol) in THF was added 15 ml of TBAF (1M solution in THF) at 0° C. The resulting mixture was stirred at room temperature for 2 hours. After the completion of the reaction, THF was removed partially and the residue was extracted with ethyl acetate, washed with water and brine. The organic layers were dried over Na₂SO₄ and the volatiles were removed under reduced pressure. The crude product was purified by column chromatography to obtain the desired product (5.0 grams).

Yield: 96%

IR (KBr, cm⁻¹): 3242, 2962, 1749, 1666, 1529, 1415, 1213, 748

¹H NMR (400 MHz, DMSO): δ 8.26 (t, J=5.8 Hz, 1H), 7.92 (s, 1H), 7.72 (dd, J=2.4, 13.7 Hz, 1H), 7.65 (t, J=8.9 Hz, 1H), 7.45 (dd, J=1.9, 8.9 Hz, 1H), 7.34 (s, 1H), 4.97 (t, J=5.6 Hz, 1H), 4.79-4.75 (m, 1H), 4.41 (d, J=5.4 Hz, 2H), 4.17 (t, J=8.9 Hz, 1H), 3.79 (dd, J=6.4, 9.4 Hz, 1H), 3.43 (t, J=5.4 Hz, 2H), 1.84 (s, 3H)

ES-MS (m/z): 349 (M⁺+1)

### Preparation 78

### (S)-N-{3-[4-(4-Cyanomethyl-imidazol-1-yl)-3-fluoro-phenyl]-2-oxo-oxazolidin-5-ylmethyl}-acetamide

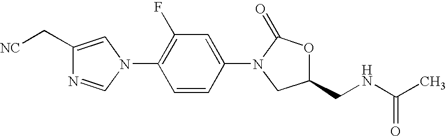

To a solution of N-{3-[3-Fluoro-4-(4-hydroxymethyl-imidazol-1-yl)-phenyl]-2-oxo-oxazolidin-5-ylmethyl}-acetamide (5 grams, 14.4 mmol) in dry DCM was added triethyl amine (8.7 grams, 86.1 mmol) and methanesulphonyl chloride (3.0 grams, 26.0 mmol) at 0° C. and stirred at room temperature for 2 hours. Reaction mixture was extracted with DCM and concentrated. The crude compound was dissolved in dry DMF and potassium cyanide (1.87 grams, 29.0 mmol) was added and heated to 70° C. for 6 hours. The reaction mixture was diluted with ethyl acetate and washed with water and brine. The organic layers were dried over Na₂SO₄ and evaporated under vacuum. The crude compound was purified by column chromatography to afford the desired compound as a yellow solid (1 gram).

Yield: 29%

IR (Neat, cm⁻¹): 2924, 2853, 1753, 1664, 1528, 1412, 1211

1H NMR (400 MHz, DMSO): 8.22 (t, J=5.6 Hz, 1H), 8.01 (s, 1H), 7.73 (dd, J=2.4, 13.7 Hz, 1H), 7.67 (t, J=8.9 Hz, 1H), 7.48 (s, 1H), 7.46 (dd, J=1.6, 8.9 Hz, 1H), 4.77-4.75 (m, 1H), 4.17 (t, J=9.1 Hz, 1H), 3.93 (s, 2H), 3.78 (dd, J=6.4, 9.1 Hz, 1H), 3.43 (t, J=5.6 Hz, 2H), 1.84 (s, 3H).

ES-MS (m/z): 358 (M⁺+1)

### Preparation 79

### (S)-N-{3-[3-Fluoro-4-(4-thiocarbamoylmethyl-imidazol-1-yl)-phenyl]-2-oxo-oxazolidin-5-ylmethyl}-acetamide

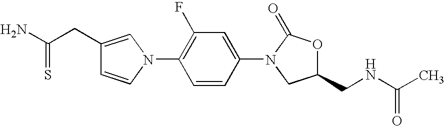

To a solution of N-{3-[4-(4-Cyanomethyl-imidazol-1-yl)-3-fluoro-phenyl]-2-oxo-oxazolidin-5-ylmethyl}-acetamide (400 mg, 1.12 mmol)) in dry DMF was passed hydrogen sulphide gas at 100° C. for overnight. The reaction mixture was diluted with ethyl acetate, washed with water and brine. The combined organic layers were dried over Na₂SO₄ and purified by column chromatography to get the desired product as a yellow solid (200 mg)

Yield: 46%

1H NMR (400 MHz, DMSO): δ 9.76 (brs, 1H), 9.49 (bs, 1H), 9.24 (bs, 1H), 8.27 (t, J=5.6 Hz, 1H), 7.85-7.77 (m, 3H), 7.53 (dd, J=1.6, 8.9 Hz, 1H), 4.81-4.76 (m, 1H), 4.19 (t, J=8.9 Hz, 1H), 4.02 (s, 2H), 3.82 (dd, J=6.4, 9.1 Hz, 2H), 3.61-3.16 (m, 1H), 1.84 (s, 3H).

ES-MS (m/z): 392 (M⁺+1)

### Preparation 80

### (S)-N-[3-(3-Fluoro-4-hydrazinothiocarbonyl-phenyl)-2-oxo-oxazolidin-5-ylmethyl]-acetamide

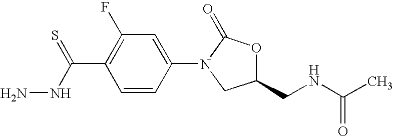

The above compound was synthesized using the procedure mentioned in Bioorg. Med. Chem. Lett., 2003, 13, 4193-4196

### Preparation 81

### (S)-N-{3-[4-(5-Chloromethyl-[1,3,4]thiadiazol-2-yl)-3-fluoro-phenyl]-2-oxo-oxazolidin-5-ylmethyl}-acetamide

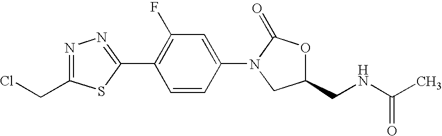

To a solution of N-[3-(3-Fluoro-4-hydrazinothiocarbonyl-phenyl)-2-oxo-oxazolidin-5-ylmethyl]-acetamide (75 mg, 0.23 mmol) in dry THF was added chloroacetyl chloride (65 mg, 0.58 mmol) dropwise and refluxed for 1 hour. After the completion of the reaction, solvent was removed and the residue was purified by column to afford the title product as a white solid (80 mg)

Yield: 91%

IR (KBr, cm⁻¹): 3305, 1742, 1543, 1419, 1219, 1080, 870, 756

¹H NMR (400 MHz, DMSO): δ 8.30 (t, J=8.9 Hz, 1H), 8.23 (t, J=5.9 Hz, 1H), 7.76 (dd, J=2.1, 13.7 Hz, 1H), 7.56 (dd, J=2.4, 8.9 Hz, 1H), 5.32 (s, 2H), 4.82-4.76 (m, 1H), 4.23 (t, J=9.1 Hz, 1H), 3.82 (dd, J=6.5, 9.1 Hz, 1H), 3.45 (t, J=5.6 Hz, 2H), 1.84 (s, 3H).

ES-MS (m/z): 385 (M⁺+1)

### Preparation 82

### (S)-N-{3-[3-Fluoro-4-(4-hydroxymethyl-[1,2,3]triazol-1-yl)-phenyl]-2-oxo-oxazolidin-5-ylmethyl}-acetamide

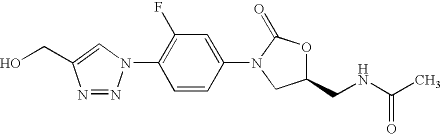

Following the general procedure the titled compound was synthesized from N-[3-(4-Azido-3-fluoro-phenyl)-2-oxo-oxazolidin-5-ylmethyl]-acetamide.

Yield: 84%

IR (KBr, cm⁻¹): 2936, 2676, 2493, 2360, 1752, 1528, 1475, 1398, 1036.

¹H NMR (400 MHz, DMSO): δ 8.40 (d, J=1.8 Hz, 1H), 8.24 (t, J=5.6 Hz, 1H), 7.83 (t, J=8.6 Hz, 1H), δ 7.79 (s, 2H), 4.19 (t, J=9.1 Hz, 1H), 3.82 (dd, 6.4 Hz, 9.1 Hz, 1H), 3.43 (t, J=5.6 Hz, 2H), 1.84 (s, 3H)

ES-MS (m/z): 350 (M⁺+1)

### Preparation 83

### (S)-N-{3-[4-(4-Cyanomethyl-[1,2,3]triazol-1-yl)-3-fluoro-phenyl]-2-oxo-oxazolidin-5-ylmethyl}-acetamide

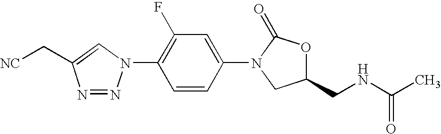

The title compound is prepared by following the procedure as described in preparation 78, by taking appropriate starting materials.

Yield: 76%

IR (KBr, cm⁻¹): 3351, 1756, 1668, 1525, 1416, 1203, 1049, 1009, 751, 686.

1H NMR (400 MHz, DMSO): δ 8.56 (d, J=1.8 Hz, 1H), 8.23 (t, J=5.9 Hz, 1H), 7.86 (t, J=8.8 Hz, 1H), 7.81 (dd, J=2.4 Hz, 13.4 Hz, 1H), 7.56-7.53 (m, 1H), 4.84-4.75 (m, 1H), 4.23 (s, 2H), 4.20 (t, J=8.9 Hz, 1H), 3.88-3.78 (m, 1H), δ 3.44 (t, J=5.6 Hz, 2H), 1.84 (s, 3H).

ES-MS (m/z): 359 (M⁺+1)

### Preparation 84

### (S)-N-(3-{3-Fluoro-4-[4-(N-hydroxycarbamimidoylmethyl)-[1,2,3]triazol-1-yl]-phenyl}-2-oxo-oxazolidin-5-ylmethyl)-acetamide

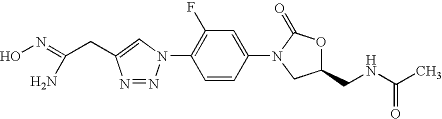

To a solution of N-{3-[4-(4-Cyanomethyl-[1,2,3]-triazol-1-yl)-3-fluoro-phenyl]-2-oxo-oxazolidin-5-ylmethyl}-acetamide (300 mg, 0.837 mmol) and hydroxylamine hydrochloride (230 mg, 3.34 mmol) in ethanol, a solution of Na₂CO₃ (266 mg, 2.51 mmol) was added dropwise and the reaction mixture was stirred at 65° C. for overnight. The solvent was removed under vacuum and the residue was purified by column chromatography to obtain the required product as a white solid (300 mg).

Yield: 92%

IR (KBr, cm⁻¹): 2974, 2937, 2739, 2677, 2491, 1732, 1660, 1529, 1475, 1433, 1399, 1243, 1169, 1131, 1036, 713.

1H NMR (400 MHz, DMSO): δ 10.15 (bs, 1H), 8.34 (d, J=1.8 Hz, 1H), 8.27 (t, J=5.9 Hz, 1H), 7.90-7.76 (m, 2H), 7.53 (dd, J=1.6 Hz, 9.1 Hz, 1H), 5.70 (bs, 1H), 4.84-4.75 (m, 1H), 4.19 (t, J=8.8 Hz, 1H), 3.83 (dd, J=6.4 Hz, 9.2 Hz, 1H), 3.51 (s, 2H), 3.44 (t, J=5.3 Hz, 2H), 1.84 (s, 3H).

ES-MS (m/z): 392 (M⁺+1)

### Example 1

### 5-(R)-azidomethyl-3-[3-fluoro-4-(4-pyrazol-1-ylmethyl-oxazol-2-yl)-phenyl]-oxazolidin-2-one

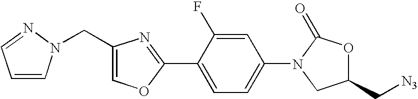

A solution of 5-(R)-azidomethyl-3-[4-(4-chloromethyl-oxazol-2-yl)-3-fluoro-phenyl]-oxazolidin-2-one (0.5 grams, 1.43 mmol) in acetonitrile (15 mL) was treated with pyrazole (0.2 grams, 2.85 mmol) in presence of anhydrous potassium carbonate (0.6 grams, 4.28 mmol) at 85° C. for 20 hours. Solids were filtered off and filtrate was evaporated. The residue obtained was dissolved in ethyl acetate (100 mL) and it was washed with water (50 mL) and brine solution successively. Finally the organic layer was dried over anhydrous sodium sulfate and the volatiles were evaporated. The residue obtained was purified by column chromatography by using basic alumina in 0.2% methanol:chloroform system.

Yield: 34%,

¹H NMR (300 MHz, CDCl₃): δ 8.01 (t, J=8.4 Hz, 1H), 7.51-7.65 (m, 4H), 7.35 (dd, J=2.0 & 6.7 Hz, 1H), 6.30 (s, 1H), 6.88-6.76 (m, 1H), 4.12 (t, J=8.9 Hz, 1H), 3.90 (dd, J=2.6 & 6.3 Hz, 1H), 3.70 (dq, J=4.4 & 13.3 Hz, 2H).

### Example 2

### N-{3-[3-fluoro-4-(4-pyrazol-1-ylmethyl-oxazol-2-yl)-phenyl]-2-oxo-oxazolidin-5(S)-ylmethyl}-acetamide

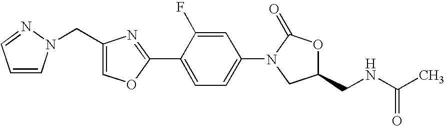

To a solution of 5-(R)-azidomethyl-3-[3-fluoro-4-(4-pyrazol-1-ylmethyl-oxazol-2-yl)-phenyl]-oxazolidin-2-one (185 mg, 0.53 mmol) in tetrahydrofuran (5 mL) was added triphenyl phosphene (0.28 grams, 1.0 mmol) and stirred at room temperature for 2 hours. It was then heated to 75° C. for 1.5 hours after the addition of water (400 μl). Solvent was evaporated and the residue obtained was dissolved in pyridine (4 mL). Acetic anhydride (150 μl) was added to it at 0° C. and stirred for 30 minutes. Pyridine was removed and the residue obtained was purified by column chromatography by using basic alumina in 1.2% methanol:chloroform.

Yield: 40%,

MS (m/z): 400 (M⁺+1), 332, 279,

Melting Range: 204-206° C.,

¹H NMR (300 MHz, DMSO-d₆): δ 8.3 (t, J=5.7 Hz, 1H), 8.18 (s, 1H), 8.02 (t, J=8.8 Hz, 1H), 7.82 (d, J=1.9 Hz, 1H), 7.68-7.62 (dd, J=2.3 & 13.8 Hz, 1H), 7.50 (dd, J=2.2 & 13.8 Hz, 1H), 7.45 (dd, J=0.7 & 1.9 Hz, 1H), 6.31 (dt, J=2.1 & 4.1 Hz, 1H), 5.3 (s, 2H), 4.72-4.82 (m, 1H), 4.25 (t, J=9.2 Hz, 1H); 3.80 (dd, J=2.8 & 6.5 Hz, 1H), 3.42 (d, J=6.7 Hz, 1H), 1.82 (s, 3H).

### Example 3

### 5-(R)-azidomethyl-3-[3-fluoro-4-(4-[1,2,4]triazol-1-ylmethyl-oxazol-2-yl)-phenyl]-oxazolidin-2-one

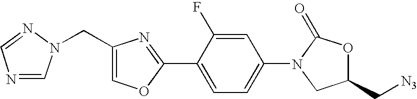

A solution of 5-(R)-azidomethyl-3-[4-(4-chloromethyl-oxazol-2-yl)-3-fluoro-phenyl]-oxazolidin-2-one (0.5 grams, 1.4 mmol) in acetonitrile (15 mL) was treated with triazole (0.2 grams, 2.9 mmol) in presence of anhydrous potassium carbonate (0.6 grams, 4.3 mmol) at 85° C. for 20 hours. Solids were filtered off and filtrate was evaporated. The residue obtained was dissolved in ethyl acetate (100 mL) and it was washed with water (50 mL) and brine solution successively. Finally the organic layer was dried over anhydrous sodium sulfate and the volatiles were evaporated. The residue obtained was purified by column chromatography by using basic alumina in 0.2% methanol:chloroform system.

Yield: 40%,

¹H NMR (300 MHz, CDCl₃): δ 8.30 (s, 1H), 8.02 (d, J=8.5 Hz, 1H), 7.98 (s, 1H), 7.75 (s, 1H), 7.62 (dd, J=2.1 & 10.9 Hz, 1H), 7.35 (dd, J=2.2 & 6.6 Hz, 1H), 5.48 (s, 2H), 4.78-4.88 (m, 1H), 4.12 (t, J=9.0 Hz, 1H), 3.81 (dd, J=2.7 & 8.2 Hz, 1H), 3.68 (dq, J=4.4 & 13.3 Hz, 2H).

### Example 4

### N-{3-[3-fluoro-4-(4-[1,2,4]-triazol-1-ylmethyl-oxazol-2-yl)-phenyl]-2-oxo-oxazolidin-5(S)-ylmethyl}-acetamide

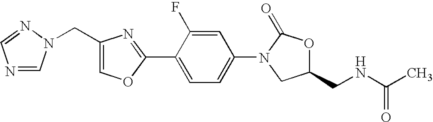

To a solution of 5-(R)-azidomethyl-3-[3-fluoro-4-(4-[1,2,4]triazol-1-ylmethyl-oxazol-2-yl)-phenyl]-oxazolidin-2-one (200 mg, 0.5 mmol) in tetrahydrofuran (4 mL) was added triphenyl phosphene (0.28 grams, 1.0 mmol) and stirred at room temperature for 2 hours. It was heated to 75° C. for 1.5 hours after the addition of water (400 μl). Solvent was evaporated and the residue obtained was dissolved in pyridine (4 mL). Acetic anhydride (150 μl) was added to it at 0° C. and stirred for 30 minutes. Pyridine was removed and the residue obtained was purified by column chromatography by using basic alumina in 1.2% methanol:chloroform.

Yield: 63%,

Melting Range: 160-162° C.,

MS (m/z): 401 (M⁺+1), 332,

¹H NMR (300 MHz, DMSO-d₆): δ 8.64 (s, 1H), 8.26 (bs, 2H), 7.95-8.02 (m, 2H), 7.72-7.40 (m, 2H), 5.43 (s, 2H), 4.89-4.70 (m, 1H), 4.17 (t, J=8.9 Hz, 1H), 3.80 (t, J=6.7 Hz, 1H), 3.43 (t, J=5.1 Hz, 2H), 1.84 (s, 3H).

### Example 5

### 5-(R)-azidomethyl-3-[3-fluoro-4-(4-imidazol-1-ylmethyl-oxazol-2-yl)-phenyl]-oxazolidin-2-one

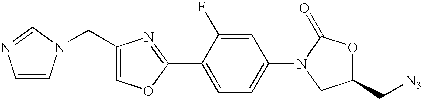

The title compound is prepared by following the procedure as described in example 3, by taking appropriate starting materials.

MS (m/z): 384 (M⁺+1), 356.

### Example 6

### Preparation of N-{3-[3-fluoro-4-(4-imidazol-1-ylmethyl-oxazol-2-yl)-phenyl]-2-oxo-oxazolidin-5(S)-ylmethyl}-acetamide

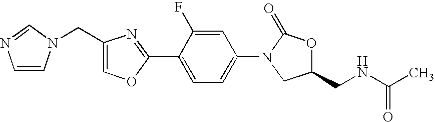

The title compound is prepared by following the procedure as described in example 4, by taking appropriate starting materials.

Yield: 42%,

Melting Range: 198-200° C.,

MS (m/z): 400 (M⁺+1), 332, 279,

¹H NMR (DMSO-d₆): δ 8.25 (t, J=5.8 Hz, 1H), 8.20 (s, 1H), 8.00 (t, J=8.7 Hz, 1H), 7.74 (s, 1H), 7.45 (dd, J=2.1 & 13.8 Hz, 1H), 7.50 (dd, J=2.1 & 8.8 Hz, 1H), 7.22 (bs, 1H), 6.91 (s, 1H), 5.20 (s, 2H), 4.82-4.72 (m, 1H), 4.16 (t, J=9.0 Hz, 1H), 3.79 (dd, J=6.5 & 9.3 Hz, 1H), 3.42 (t, J=5.5 Hz, 2H), 1.82 (s, 3H).

### Example 7

### Tert-butyl {3-[3-tluoro-4-(4-imidazol-1-ylmethyl-thiazol-2-yl)-phenyl]-2-oxo-oxazolidin-5(S)-ylmethyl}-carbamate

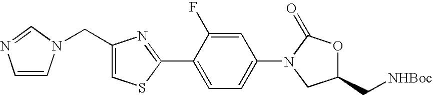

A mixture of tert-butyl {3-[4-(4-chloromethyl-thiazol-2-yl)-3-fluoro-phenyl]-2-oxo-oxazolidin-5(S)-ylmethyl}-carbamate (3 grams, 7.79 mmol), potassium carbonate (4.3 grams, 31.16 mmol) and imidazole (1.2 grams, 15.58 mmol) in acetonitrile (100 mL) was heated to 75° C. for 4 hours. Potassium carbonate was separated by filtration and the filtrate concentrated. The residue obtained was extracted with chloroform. The chloroform layer was dried over sodium sulfate and concentrated to obtain a brown solid.

Yield: 89%,

¹H NMR (300 MHz, CDCl₃): δ 8.24 (t, J=8.6 Hz, 1H), 7.72-7.66 (m, 2H), 7.28-7.24 (m, 1H), 7.11-7.06 (m, 3H), 5.31 (s, 2H), 4.98 (bs, 1H), 4.83-4.78 (m, 1H), 4.09 (t, J=8.0 Hz, 1H), 3.92 (dd, J=6.4 & 9.1 Hz, 1H), 3.56 (dd, J=4.3 & 6.3 Hz, 2H), 1.42 (s, 9H).

### Example 8

### Tert-butyl {3-[3-fluoro-4-(4-[1,2,4]triazol-1-ylmethyl-thiazol-2-yl)-phenyl]-2-oxo-oxazolidin-5(S)-ylmethyl}-carbamate

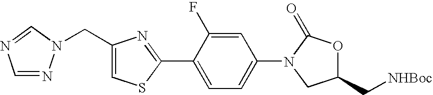

The title compound is prepared by following the procedure as described in example 7, by taking appropriate starting materials.

Yield: 63%,

¹H NMR (300 MHz, CDCl₃): δ 8.30 (s, 1H), 8.22 (t, J=8.6 Hz, 1H), 7.99 (s, 1H), 7.70 (dd, J=2.2 & 13.5 Hz, 1H), 7.29-7.22 (m, 2H), 5.54 (s, 2H), 5.04 (s, 2H), 5.04 (bs, 1H), 4.83-4.78 (m, 1H), 4.08 (t, J=9.0 Hz, 1H), 3.91 (dd, J=6.6 & 8.8 Hz, 1H), 3.56 (dd, J=4.3 & 6.3 Hz, 2H), 1.41 (s, 9H).

### Example 9

### Tert-butyl {3-[3-fluoro-4-(4-pyrazol-1-ylmethyl-thiazol-2-yl)-phenyl]-2-oxo-oxazolidin-5(S)-ylmethyl}-carbamate

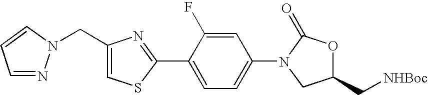

The title compound is prepared by following the procedure as described in example 7, by taking appropriate starting materials.

Yield: 83.3%.

¹H NMR (300 MHz, CDCl₃): δ 8.24 (t, J=8.6 Hz, 1H), 7.65 (dd, J=2.2 & 13.5 Hz, 1H), 7.60 (d, J=2.3 Hz, 1H), 7.57 (d, J=1.5 Hz, 1H), 7.48-7.24 (m, 1H), 7.09 (s, 1H), 6.32 (t, J=2.1 Hz, 1H), 5.53 (s, 2H), 5.05 (bs, 1H), 4.82-4.77 (m, 1H), 4.07 (t, J=9.0 Hz, 1H), 3.9 (dd, J=6.7 & 8.8 Hz, 1H), 3.55 (dd, J=4.3 & 6.0 Hz, 2H), 1.42 (s, 9H).

### Example 10

### N-{3-[3-fluoro-4-(4-imidazol-1-ylmethyl-thiazol-2-yl)-phenyl]-2-oxo-oxazolidin-5(S)-ylmethyl}-acetamide

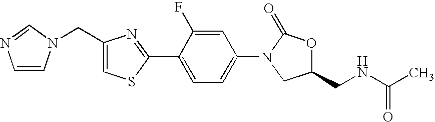

Tert-Butyl-{3-[3-fluoro-4-(4-imidazol-1-ylmethyl-thiazol-2-yl)-phenyl]-2-oxo-oxazolidin-5(S)-ylmethyl}-carbamate (0.5 grams, 1.05 mmol) and trifluoroacetic acid (2.5 mL, 5.27 mmol) were stirred in dichloromethane for 1 hour. Dichloromethane and trifluoroacetic acid were completely evaporated and diethyl ether was added to it. The solid thus obtained was dissolved in dichloromethane (10 mL) and pyridine (0.56 mL) was added followed by the addition of acetic anhydride (0.162 mL, 1.6 mmol) at 0° C. The reaction mixture was stirred at room temperature for 1 hour. Dichloromethane and pyridine was evaporated and the residue obtained after addition of water was collected. Pure product (0.18 grams) was obtained after purification by column chromatography over basic alumina using 2% methanol in chloroform as eluent.

Yield: 42%

Melting Range: 192-194° C.

MS (m/z): 416 (M⁺+1), 348,

¹H NMR (300 MHz, CDCl₃): δ 8.27 (t, J=5.7 Hz, 1H), 8.20 (t, J=8.8 Hz, 1H), 7.79 (s, 1H), 7.69 (dd, J=2.1 & 14.2 Hz, 1H), 7.59 (s, 1H), 7.50 (dd, J=2.1 & 8.8 Hz, 1H), 7.26 (s, 1H), 6.92 (s, 1H), 5.38 (s, 2H), 4.80-4.75 (m, 1H), 4.17 (t, J=9.0 Hz, 1H), 3.79 (dd, J=6.6 & 9.2 Hz, 1H), 3.43 (t, J=5.4 Hz, 2H), 1.82 (s, 3H).

### Example 11

### N-{3-[3-fluoro-4-(4-[1,2,4]triazol-1-ylmethyl-thiazol-2-yl)-phenyl]-2-oxo-oxazolidin-5(S)-ylmethyl}-acetamide

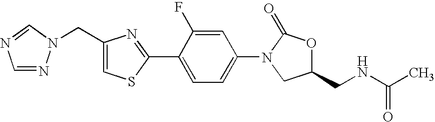

Tert-Butyl-{3-[3-fluoro-4-(4-[1,2,4]triazol-1-ylmethyl-thiazol-2-yl)-phenyl]-2-oxo-oxazolidin-5(S)-ylmethyl}-carbamate (0.5 grams, 1.05 mmol) and trifluoroacetic acid (2.5 mL) were stirred in dichloromethane for 1 hour. Dichloromethane and trifluoroacetic acid were completely evaporated and diethyl ether was added to it. The solid thus obtained was dissolved in dichloromethane (10 mL) and triethylamine (0.56 mL) was added followed by the addition of acetic anhydride (0.16 mL, 1.6 mmol) at 0° C. The reaction mixture was stirred at room temperature for 1 hour. Dichloromethane and triethylamine was evaporated and the residue obtained after addition of water was collected. Pure product (0.08 grams) was obtained after purification by column chromatography over basic alumina using 2% methanol in chloroform as eluent.

Yield: 22%

Melting Range: 196-198° C.,

MS (m/z): 417 (M⁺+1), 348, 304,

¹H NMR (300 MHz, DMSO-d₆): δ 8.70 (s, 1H), 8.26 (t, J=5.8 Hz, 1H), 8.16 (t, J=8.8 Hz, 1H), 8.01 (s, 1H), 7.69 (dd, J=2.1 & 14.1 Hz, 1H), 7.69 (s, 1H), 7.50 (dd, J=2.1 & 8.8 Hz, 1H), 5.61 (s, 2H), 4.80-4.75 (m, 1H), 4.17 (t, J=9.1 Hz, 1H), 3.79 (dd, J=6.6 & 9.2 Hz, 1H), 3.43 (t, J=5.4 Hz, 2H), 1.84 (s, 3H).

### Example 12

### N-{3-[3-fluoro-4-(4-pyrazol-1-ylmethyl-thiazol-2-yl)-phenyl]-2-oxo-oxazolidin-5(S)-ylmethyl}-acetamide

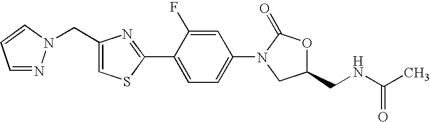

Tert-Butyl-{3-[3-fluoro-4-(4-pyrazol-1-ylmethyl-thiazol-2-yl)-phenyl]-2-oxo-oxazolidin-5(S)-ylmethyl}-carbamate (0.5 grams, 1.05 mmol) and trifluoroacetic acid (2.5 mL) were stirred in dichloromethane for 1 hour. Dichloromethane and trifluoroacetic acid were completely evaporated and diethyl ether was added to it. The solid thus obtained was dissolved in ethyl acetate (25 mL) and pyridine (0.5 mL) was added followed by the addition of acetic anhydride (0.25 mL) at 0° C. The reaction mixture was stirred at room temperature for 3 hours. Water was added to the reaction mixture and ethyl acetate layer separated. Water layer was extracted with ethyl acetate additional 2 times. Combined ethyl acetate portion was dried over sodium sulfate and concentrated. Pure product (0.1 grams, 15%) was obtained after purification by column chromatography over basic alumina using 1% methanol in chloroform as eluent.

Melting Range: 178-180° C.,

MS (m/z): 416 (M⁺+1), 348, 304,

¹H NMR (300 MHz, DMSO-d₆): δ 8.27 (t, J=5.6 Hz, 1H), 8.18 (t, J=8.8 Hz, 1H), 4.37 (d, J=2.2 Hz, 1H), 7.69 (dd, J=2.1 & 14.2 Hz, 1H), 7.51 (d, J=2.2 Hz, 1H), 7.50 (s, 1H), 7.48 (d, J=1.8 Hz, 1H), 6.30 (t, J=2.1 Hz, 1H), 5.51 (s, 2H), 4.81-4.72 (m, 1H), 4.17 (t, J=9.0 Hz, 1H), 3.79 (dd, J=6.6 & 9.2 Hz, 1H), 3.43 (t, J=5.4 Hz, 2H), 1.82 (s, 3H).

### Example 13

### N-{3-[3-fluoro-4-(2-pyrazol-1-ylmethyl-thiazol-4-yl)-phenyl]-2-oxo-oxazolidin-5-ylmethyl}-acetamide

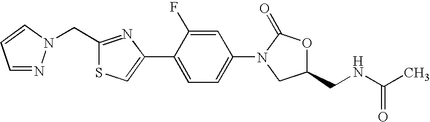

To a solution of N-{3-[4-(2-bromo-acetyl)-3-fluoro-phenyl]-2-oxo-oxazolidin-5-ylmethyl}-acetamide (300 mg, 0.8 mmol) in ethanol (2 mL) was added 2-pyrazol-1-yl-thioacetamide (113 mg, 0.8 mmol) and stirred at room temperature for 16 hours and then refluxed for additional 3 hours. The reaction mixture was diluted with ethyl acetate (100 mL), washed with saturated sodium bicarbonate solution, water and brine solution. Organic portion was then dried over sodium sulphate and concentrated. The residue obtained was washed with petroleum ether to obtain a white solid.

Yield: 90%,

IR (KBr, cm⁻¹): 3335, 2926, 1770, 1406, 1194, 1048, 751 and 636,

MS (m/z): 416 (M⁺+1), 372,

¹H NMR (200 MHz, CDCl₃): δ 8.19 (t, J=8.6 Hz, 1H), 7.67-7.57 (m, 4H), 7.26-7.21 (m, 1H), 6.35 (s, 1H), 6.05 (bs, 1H), 5.69 (s, 2H), 4.79 (bs, 1H), 4.14-4.05 (m, 1H), 3.85-3.64 (m, 3H), 2.03 (s, 3H).

### Example 14

### N-{3-[3-fluoro-4-(2[1,2,4]triazol-1-ylmethyl-thiazol-4-yl)-phenyl]-2-oxo-oxazolidin-5-ylmethyl}-acetamide

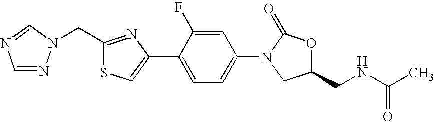

The title compound is prepared by following the procedure as described in example 13, by taking appropriate starting materials.

Melting Range: 154-156° C.,

IR (KBr, cm⁻¹): 3273, 1740 and 1410,

MS (m/z): 417 (M⁺+1), 373,

¹H NMR (200 MHz, DMSO-d₆+CDCl₃): δ 8.25-8.12 (m, 2H), 7.95-7.80 (m, 3H), 7.32-7.20 (m, 2H), 7.05 (s, 1H), 5.60 (s, 2H), 4.92-4.72 (m, 1H), 4.13 (t, J=9.0 Hz, 1H), 3.95-3.81 (m, 1H), 3.24 (bs, 2H), 1.94 (s, 3H).

### Example 15

### N-{3-[3-Fluoro-4-(2-tetrazol-1-ylmethyl-thiazol-4-yl)-phenyl]-2-oxo-oxazolidin-5-ylmethyl}-acetamide

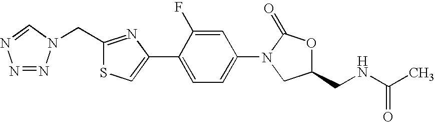

The title compound is prepared by following the procedure as described in example 13, by taking appropriate starting materials.

Melting Range: 164-166° C.,

IR (KBr, cm⁻¹): 1742, 1656, 1410 and 1226,

MS (m/z): 374 (M⁺-43), 350, 306, 304 and 261,

¹H NMR (200 MHz, CDCl₃+DMSO-d₆): δ 9.33 (s, 1H), 8.22-8.03 (m, 2H), 7.78 (s, 1H), 7.63 (d, J=14.2 Hz, 1H), 7.27 (d, J=8.8 Hz, 1H), 6.12 (s, 2H), 4.82 (bs, 1H), 4.12 (t, J=8.8 Hz, 1H), 3.87 (d, J=8.8 Hz, 1H), 3.59 (bs, 2H), 1.96 (s, 3H).

### Example 16

### N-{3-[3-Fluoro-4-(2-imidazol-1-ylmethyl-thiazol-4-yl)-phenyl]-2-oxo-oxazolidin-5-ylmethyl}-acetamide

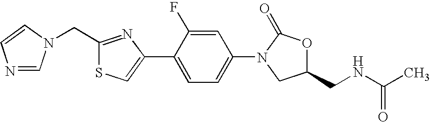

The title compound is prepared by following the procedure as described in example 13, by taking appropriate starting materials.

Melting Range: 154-156° C.,

IR (KBr, cm⁻¹): 1758, 1655 and 1407,

MS (m/z): 416 (M⁺+1), 372, 219 and 108.

¹H NMR (200 MHz, CDCl₃+DMSO-d₆): δ 8.25-8.05 (m, 2H), 7.81 (d, J=4.9 Hz, 1H), 7.78-7.52 (m, 2H), 7.29 (d, J=8.3 Hz, 1H), 7.21 (s, 1H), 7.05 (s, 1H), 5.60 (s, 2H), 4.82 (bs, 1H), 4.13 (t, J=8.8 Hz, 1H), 3.87 (t, J=8.8 Hz, 1H), 3.56 (bs, 2H), 1.94 (s, 3H).

### Example 17

### 5-(R)-azidomethyl-3-[3-fluoro-4-(4-imidazol-1-ylmethyl-pyrazol-1-yl)-phenyl]-oxazolidin-2-one

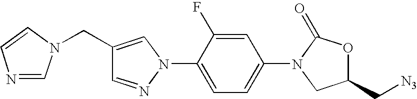

To a solution of 5-(R)-azidomethyl-3-[4-(4-chloromethyl-pyrazol-1-yl)-3-fluoro-phenyl]-oxazolidin-2-one (1.2 grams, 3.43 mmol) in acetonitrile (25 mL) were added potassium carbonate (1.89 grams, 13.71 mmol), potassium iodide (0.2 grams, catalytic amount) and imidazole (0.47 grams, 6.86 mmol). The reaction mixture was heated to 70° C. for two and half hours. Solvent was removed on a rotavapor and water (100 mL) was added. It was extracted with ethyl acetate and the combined ethyl acetate layer was washed with water (50 mL×1) and brine (50 mL×1) successively. Organic layer was concentrated and the residue formed was purified by column chromatography (Basic alumina) with chloroform and methanol. Product came out at 0.5-1% methanol in chloroform

Yield: 49.6%,

MS (m/z): 384 (M⁺+1),

¹H NMR (300 MHz, CDCl₃): δ 7.83-7.94 (m, 2H), 7.77 (dd, J=2.4 & 13.9 Hz, 1H), 7.65 (s, 1H), 7.58 (s, 1H), 7.22-7.28 (m, 1H), 7.10 (s, 1H), 6.98 (s, 1H), 5.11 (s, 2H), 4.78-4.88 (m, 1H), 4.07-4.16 (m, 1H), 3.90 (dd, J=2.6 & 6.2 Hz, 1H), 3.69 (dq, J=4.4 & 13.2 Hz, 1H).

### Example 18

### N-{3-[3-fluoro-4-(4-imidazol-1-ylmethyl-pyrazol-1-yl)phenyl]-2-oxo-oxazolidin-5-(8)-ylmethyl}-acetamide

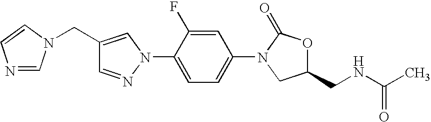

To a solution of 5-(R)-azidomethyl-3-[3-fluoro-4-(4-pyrazol-1-ylmethyl-pyrazol-1-yl)-phenyl]-oxazolidin-2-one (0.3 grams, 0.79 mmol) in tetrahydrofuran (10 mL) was added triphenylphosphene (0.27 grams, 1.02 mmol) in small batches at room temperature and stirring was continued for 2 hours. Water (1 mL) was added and heated to 75° C. for 4 hours. Solvent was removed by azeotropic distillation with benzene and the residue obtained was dissolved in pyridine (5 mL). Acetic anhydride (210 μl, 1.56 mmol) was added to it at ice temperature and stirring was continued for ½ hour. The reaction mixture was diluted with water and then extracted with ethyl acetate. Ethyl acetate portion was washed with 1N hydrochloric acid, water and brine successively. Finally the organic layer was dried over sodium sulfate and concentrated. The title compound was obtained after column chromatographic purification.

Yield: 27%,

Melting Range: 171-172° C.,

MS (m/z): 399 (M⁺+1),

¹H NMR (300 MHz, CDCl₃): δ 7.94 (d, J=2.3 Hz, 1H), 7.86 (t, J=8.9 Hz, 1H), 7.73 (dd, J=2.4 & 13.9 Hz, 1H), 7.65 (s, 1H), 7.59 (s, 1H), 7.22 (dd, J=1.4 & 8.9 Hz, 1H), 7.09 (s, 1H), 6.97 (s, 1H), 6.29-6.33 (m, 1H), 5.10 (s, 2H), 4.80-4.85 (m, 1H), 4.08 (t, J=9.0 Hz, 1H); 3.82 (dd, J=2.2 & 6.8 Hz, 1H), 3.64-3.71 (m, 2H), 2.03 (s, 3H).

### Example 19

### 5-azidomethyl-3-[3-fluoro-4-(4-pyrazol-1-ylmethyl-pyrazol-1-yl)-phenyl]-oxazolidin-2-one

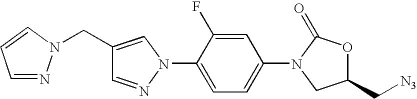

The title compound is prepared by following the procedure as described in example 17, by taking appropriate starting materials.

Yield: 57.2%,

MS (m/z): 384 (M⁺+1),

¹H NMR (300 MHz, CDCl₃): δ 7.98 (d, J=2.5 Hz, 1H), 7.87 (t, J=8.8 Hz, 1H), 7.75 (dd, J=2.5 & 13.9 Hz, 1H), 7.69 (s, 1H), 7.55 (d, J=1.5 Hz, 1H), 7.43 (d, J=2.1 Hz, 1H), 7.21-7.26 (m, 1H), 6.28 (t, J=2.1 Hz, 1H), 5.29 (s, 2H), 4.78-4.88 (m, 1H), 4.11 (t, J=9.0 Hz, 1H), 3.88 (dd, J=2.6 & 6.2 Hz, 1H), 3.68 (dq, J=4.5 & 13.3 Hz, 1H).

### Example 20

### N-{3-[3-fluoro-4-(4-pyrazol-1-ylmethyl-pyrazol-1-yl)-phenyl]-2-oxo-oxazolidin-5-(S)-ylmethyl}-acetamide

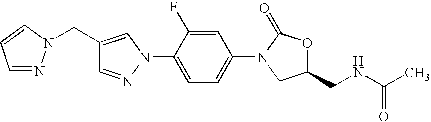

The title compound was obtained following the procedure reported for N-{3-[3-fluoro-4-(4-imidazol-1-ylmethyl-pyrazol-1-yl)phenyl]-2-oxo-oxazolidin-5-(S)-ylmethyl}-acetamide from 5-(R)-azidomethyl-3-[3-fluoro-4-(4-pyrazol-1-ylmethyl-pyrazol-1-yl)-phenyl]-oxazolidin-2-one.

Yield: 28%,

Melting Range: 186-187° C.

MS (m/z): 399 (M⁺+1),

¹H NMR (300 MHz, CDCl₃): δ 7.98 (s, 1H), 7.85 (t, J=8.8 Hz, 1H), 7.68-7.72 (m, 2H), 7.54 (d, J=1.1 Hz, 1H), 7.43 (d, J=1.5 Hz, 1H), 7.18-7.26 (m, 2H), 6.28 (t, J=1.9 Hz, 1H), 6.11 (bs, 1H), 5.29 (s, 2H), 4.80-4.85 (m, 1H), 4.08 (t, J=9.0 Hz, 1H), 3.81 (dd, J=1.6 & 7.1, 1H), 3.64-3.69 (m, 2H), 2.02 (s, 3H).

### Example 21

### 5-(R)-azidomethyl-3-[3-fluoro-4-(4-[1,2,4]triazol-1-ylmethyl-pyrazol-1-yl)-phenyl]-oxazolidin-2-one

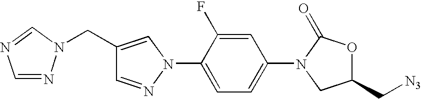

The title compound is prepared by following the procedure as described in example 17, by taking appropriate starting materials.

Yield: 61%,

MS (m/z): 384 (M⁺+1),

¹H NMR (300 MHz, CDCl₃): δ 8.11 (s, 1H), 8.05 (d, J=2.4 Hz, 1H), 7.98 (s, 1H), 7.88 (t, J=8.9 Hz, 1H), 7.72-7.81 (m, 2H), 7.21-7.28 (m, 1H), 5.34 (s, 2H), 4.78-4.88 (m, 1H), 4.12 (t, J=8.9 Hz, 1H), 3.90 (dd, J=2.6 & 6.2 Hz, 1H), 3.68 (dq, J=4.5 & 13.3 Hz, 1H).

### Example 22

### N-{3-[3-fluoro-4-(4-[1,2,4]triazol-1-ylmethyl-pyrazol-1-yl)-phenyl]-2-oxo-oxazolidin-5(S)-ylmethyl}-acetamide

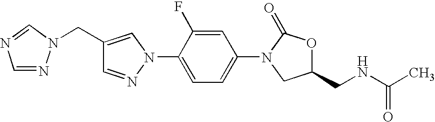

The title compound was obtained following the procedure reported for N-{3-[3-fluoro-4-(4-imidazol-1-ylmethyl-pyrazol-1-yl)phenyl]-2-oxo-oxazolidin-5-(S)-ylmethyl}-acetamide from 5-(R)-azidomethyl-3-[3-fluoro-4-(4-[1,2,4]triazol-1-ylmethyl-pyrazol-1-yl)-phenyl]-oxazolidin-2-one.

Yield: 27%,

Melting Range: 126-127° C.

MS (m/z): 400 (M⁺+1),

¹H-NMR (300 MHz, CDCl₃): δ 8.12 (s, 1H), 8.04 (d, J=2.2 Hz, 1H), 7.98 (s, 1H), 7.85 (t, J=8.9 Hz, 1H), 7.69-7.76 (m, 2H), 7.22 (dd, J=1.3 & 9.0 Hz, 1H), 6.24 (t, J=6.1 Hz, 1H), 5.34 (s, 2H), 4.76-4.86 (m, 1H), 4.08 (t, J=9.1 Hz, 1H), 3.83 (dd, J=2.2 & 6.9 Hz, 1H), 3.62-3.72 (m, 2H), 2.02 (s, 3H).

### Example 23

### 5-azidomethyl-3-[3-fluoro-4-(4-imidazol-1-ylmethyl-imidazol-1-yl)-phenyl]-oxazolidin-2-one

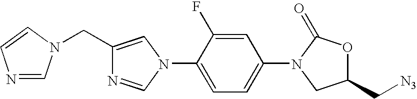

The title compound is prepared by following the procedure as described in example 17, by taking appropriate starting materials.

Yield: 45%.

¹H NMR (300 MHz, CDCl₃): δ 7.63-7.74 (m, 3H), 7.33-7.36 (m, 2H), 7.07 (s, 3H), 5.14 (s, 2H), 4.82-4.86 (m, 1H), 4.08-4.14 (m, 1H), 3.88 (dd, J=6.3 & 9.0 Hz, 1H), 3.78 (dd, J=4.4 & 13.2 Hz, 1H), 3.68 (dd, J=4.4 & 13.2 Hz, 1H).

### Example 24

### N-{3-[3-fluoro-4-(4-imidazol-1-ylmethyl-imidazol-1-yl)-phenyl]-2-oxo-oxazolidin-5-ylmethyl}-acetamide

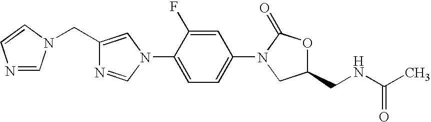

A solution of 5-azidomethyl-3-[3-fluoro-4-(4-imidazol-1-ylmethyl-imidazol-1-yl)-phenyl]-oxazolidin-2-one (0.6 grams, 1.6 mmol) in thioacetic acid (6 mL) was stirred at room temperature for 16 hours. Thiolacetic acid was removed under rotary evaporation and the residue obtained was scratched in diethyl ether (10 mL) to obtain the title compound as solid, which was collected on a Buchner funnel.

Yield: 69%,

Melting Range: 180-182° C.,

MS (m/z): 399 (M⁺+1), 331,

¹H NMR (300 MHz, CDCl₃): δ 8.02 (s, 1H), 7.78-7.86 (m, 2H), 7.42-7.64 (m, 4H), 7.22 (t, J=1.2 Hz, 1H), 7.01 (t, J=1.1 Hz, 1H), 5.22 (s, 2H), 4.82-4.90 (m, 1H), 4.22 (t, J=9.0 Hz, 1H), 3.86-3.93 (m, 1H), 3.61 (t, J=4.9 Hz, 2H), 2.00 (s, 3H).

### Example 25

### 5-Azidomethyl-3-[3-fluoro-4-(4-pyrazol-1-ylmethyl-imidazol-1-yl)-phenyl]oxazolidin-2-one

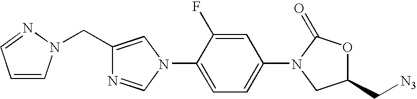

The title compound is prepared by following the procedure as described in example 17, by taking appropriate starting materials.

Yield: 35%,

¹H NMR (300 MHz, CDCl₃): δ 7.51-7.82 (m, 4H) 7.26-7.40 (m, 3H), 6.28 (t, J=2.0 Hz, 1H), 5.34 (s, 2H), 4.84-4.90 (m, 1H), 4.01 (t, J=8.9 Hz, 1H), 3.84-3.92 (m, 1H), 3.81 (dd, J=4.3 & 13.4 Hz, 1H), 3.62 (dd, J=4.1 & 13.3 Hz, 1H).

### Example 26

### N-{3-[3-fluoro-4-(4-pyrazol-1-ylmethyl-imidazol-1-yl)-phenyl]-2-oxo-oxazolidin-5-ylmethyl}-acetamide

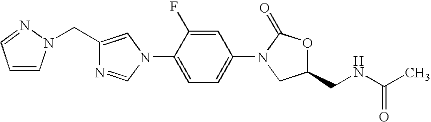

The title compound is prepared by following the procedure as described in example 18, by taking appropriate starting materials.

Yield: 51%,

Melting Range: 158-160° C.,

MS (m/z): 399 (M⁺+1), 355, 331,

¹H NMR (300 MHz, CDCl₃): δ 7.54-7.74 (m, 4H), 7.28-7.40 (m, 3H), 7.15 (s, 1H), 6.28 (bs, 1H), 5.35 (s, 2H), 4.76-4.81 (m, 1H), 4.07 (t, J=9.0 Hz, 1H), 3.66-3.87 (m, 3H), 2.05 (s, 3H).

### Example 27

### 5-Azidomethyl-3-[3-fluoro-4-(4-[1,2,4]-triazol-1-ylmethyl-imidazol-1-yl)-phenyl]-oxazolidin-2-one

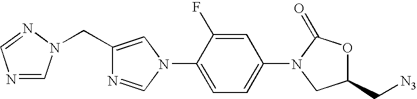

The title compound is prepared by following the procedure as described in example 17, by taking appropriate starting materials.

Yield: 58%,

¹H NMR (300 MHz, CDCl₃): δ 8.58 (s, 1H), 8.02 (s, 2H), 7.84 (dd, J=2.4 & 13.3 Hz, 1H), 7.58-7.66 (m, 2H), 7.46-7.52 (m, 1H), 5.4 (s, 2H), 4.85-4.90 (m, 1H), 4.10 (t, 1H), 3.90 (dd, J=6.0 & 8.0 Hz, 1H), 3.80 (dd, J=4.4 & 13.3 Hz, 1H), 3.68 (dd, J=4.2 & 13.2 Hz, 1H).

### Example 26

### N-{3-[3-fluoro-4-(4-[1,2,4]triazol-1-ylmethyl-imidazol-1-yl)-phenyl]-2-oxo-oxazolidin-5-ylmethyl}-acetamide

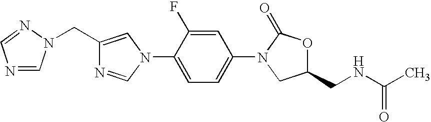

The title compound is prepared by following the procedure as described in example 18, by taking appropriate starting materials.

Yield: 48.2%,

Melting Range: 171-173° C.,

MS (m/z): 400 (M⁺+1), 331,

¹H NMR (300 MHz, CD₃OD): δ 8.56 (s, 1H), 8.01 (s, 2H), 7.84 (dd, J=2.4 & 13.3 Hz, 1H), 7.65-7.68 (m, 2H), 7.48 (d, J=9.0 Hz, 1H), 5.46 (s, 2H), 4.84-4.90 (m, 1H), 4.24 (t, J=9.0 Hz, 1H), 3.89 (dd, J=6.3 & 9.3 Hz, 1H), 3.62 (d, J=4.9 Hz, 2H).

### Example 27

### {3-[3-Fluoro-4-(4-pyrazol-1-ylmethyl-[1,2,3]triazol-1-yl)-phenyl}-5-hydroxy methyl-oxazolidin-2-one

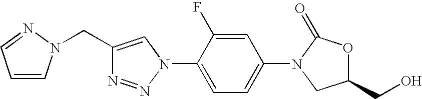

To a solution of [3-(4-azido-3-fluoro-phenyl)-5-hydroxymethyl-oxazolidin-2-one (180 mg, 0.71 mmol) in dimethylformamide (5 mL) was added N-ethyl diisopropylamine (110 mg, 0.86 mmol) followed by the addition of 1-prop-2-yne-1H-pyrazol (91 mg, 0.86 mmol) and copper iodide (67.8 mg, 0.36 mmol) at 0-5° C. and stirred well for half an hour. A saturated solution of ammonium chloride along with 2-3 drops of ammonia was then added to the reaction mixture and allowed to stir for additional 15 minutes. The reaction mixture was extracted with ethyl acetate (2×25 mL) and the combined ethyl acetate layer was washed with brine, dried over sodium sulfate and concentrated. The residue obtained was purified by column chromatography on silica gel using methanol and chloroform (1:9) as eluent to obtain the title compound as cream color solid.

Yield: 43%,

IR (KBr, cm⁻¹): 3428, 2926, 1742, 1526, 1411, 1207, 1047,

MS (m/z): 359 (M⁺+1), 330, 277, 262, 189, 175, 162, 94,

¹H NMR (DMSO-d₆, 400 MHz): δ 8.51 (d, J=1.8 Hz, 1H); 7.86-7.80 (m, 3H); 7.58-7.55 (m, 2H), 6.27 (t, J=1.3 Hz, 1H); 5.51 (s, 2H); 5.23 (t, J=6.0 Hz, 2H); 4.78-4.74 (m, 1H); 4.15 (t, J=9.1 Hz, 1H); 3.90 (dd, J=6.0 & 9.1 Hz, 1H).

### Example 28

### N-{3-[3-fluoro-4-(4-pyrazol-1-yl-methyl-(1,2,3)triazol-1-yl)-phenyl]-2-oxo-oxazolidin-5(S)-ylmethyl}acetamide

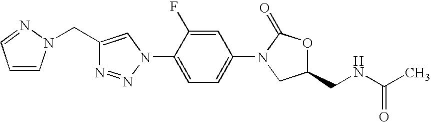

To a solution of N-[3-(4-azido-3-fluoro-phenyl)-2-oxo-oxazolidin-5-yl-methyl]-acetamide (120 mg, 0.41 mmol), 1-prop-2-yne-1H-pyrazol (120 mg, 0.41 mmol) and N-ethyl diisopropylamine (78 mg, 0.41 mmol) in dimethylformamide (10 mL) was added copper iodide (27 mg, 0.2 mmol) at 5-10° C. Stirring was continued for 0.5 hours at the same temperature and then a saturated solution of ammonium chloride along with 2-3 drops of ammonia was added. Reaction mixture was extracted with ethyl acetate (2×50 mL). Ethyl acetate layer was washed with brine, dried over sodium sulfate and concentrated. The residue obtained was purified by column chromatography on silica gel using methanol and chloroform (1:19 to 2:23) as eluent to obtain the title compound as cream color solid.

Yield: 60%,

Melting Range: 180-182° C.,

IR (KBr, cm⁻¹): 3286, 3168, 2929, 1746, 1649, 1556, 1533, 1408,

MS (m/z): 400 (M⁺+1), 357, 356, 338, 279, 208, 130,

¹H NMR (CDCl₃+DMSO-d₆, 200 MHz): δ 8.10-8.06 (m, 2H), 7.90-7.78 (m, 2H), 7.64-7.33 (m, 3H), 6.28 (bs, 1H), 5.54 (s, 2H), 4.89-4.79 (m, 1H), 4.17 (t, J=8.8 Hz, 1H), 3.83 (t, J=7.8 Hz, 1H), 3.63-3.58 (m, 2H), 1.97 (s, 2H).

### Example 29

### 5-Aminomethyl-3-[3-fluoro-4-(4-pyrazol-1-ylmethyl-[1,2,3]triazol-1-yl)-phenyl]-oxazolidin-2-one

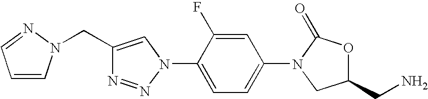

A suspension of N-{3-[3-fluoro-(4-pyrazol-1-ylmethyl-[1,2,3]triazol-1-yl)-phenyl]-2-oxo-oxazolidin-5-ylmethyl}acetamide (400 mg, 1.0 mmol) and 4N hydrochloric acid (2.5 mL) in methanol (2.5 mL) was refluxed for 10 hours. The pH of the reaction mixture was adjusted to 12 by the addition of saturated solution of sodium carbonate. It was then extracted with chloroform (40 mL×2) and the combined extract was washed with brine. Finally, it was dried over sodium sulfate and volatiles were evaporated. The residue obtained was purified by column chromatography on silica gel using methanol and chloroform (1:9) as eluent to obtain the title compound as white solid,

Yield: 26%,

IR (KBr, cm⁻¹): 3433, 2923, 1743, 1628, 1530, 1409, 1233, 1154, 1047,

MS (m/z): 358 (M⁺+1),

¹H NMR (DMSO-d₆, 200 MHz): δ 8.54 (s, 1H), 7.91-7.77 (m, 3H), 7.55-7.46 (m, 2H), 6.27 (s, 1H), 5.51 (s, 2H), 5.00-4.89 (m, 1H), 4.28 (t, J=9.2 Hz, 1H), 3.90 (t, J=7.8 Hz, 1H), 3.40 (bs, 1H), 3.34-3.15 (m, 2H).

### Example 30

### N-{3-[3-fluoro-4-(4-[1,2,4]triazol-1-ylmethyl)-[1,2,3]triazol-1-yl)-phenyl]-2-oxo-oxazolidin-5-ylmethyl}-acetamide

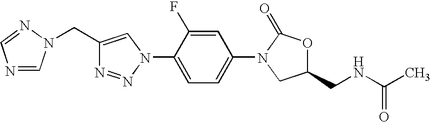

The title compound is prepared by following the procedure as described in example 28, by taking appropriate starting materials.

Yield: 70%,

Melting Range: 170-171° C.,

IR (KBr, cm⁻¹): 3285, 3135, 1732, 1658, 1472, 1244, 1135, 1051, 855, 679,

MS (m/z): 401 (M⁺+1), 372, 356, 328,

¹H NMR (DMSO-d₆, 400 MHz): δ 8.67 (s, 1H), 8.60 (d, J=1.6 Hz, 1H), 8.24 (t, J=5.7 Hz, 2H), 8.00 (s, 1H), 7.86 (t, J=8.7 Hz, 1H), 7.80 (dd, J=2.4 & 13.4 Hz, 1H) 7.54 (dd, J=1.9 & 9.1 Hz, 1H), 5.62 (s, 2H), 4.82-4.56 (m, 1H), 4.20 (t, J=9.1 Hz, 1H), 3.82 (dd, J=6.4 & 9.1 Hz, 1H), 4.45 (t, J=5.5 Hz, 2H) 1.84 (s, 3H).

### Example 31

### 5-Aminomethyl-3-[3-fluoro-4-[4-(1,2,4)triazol-1-ylmethyl)-[1,2,3]-triazol-1-yl)-phenyl]-oxazolidin-2-one

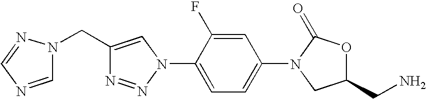

The title compound is prepared by following the procedure as described in example 29, by taking appropriate starting materials.

Yield: 21%,

### Example 32

### N-{3-[3-fluoro-4-[4-(3-trifluoromethyl-pyrazol-1-ylmethyl-[1,2,3]triazol-1-yl]-phenyl]-2-oxo-oxazolidin-5-ylmethyl}-acetamide

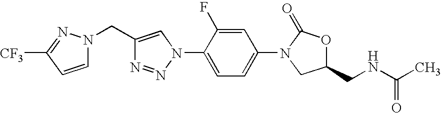

The title compound is prepared by following the procedure as described in example 28, by taking appropriate starting materials.

Yield: 38%,

Melting Range: 130-140° C.,

IR (KBr, cm⁻¹): 3296, 1735, 1660, 1531, 1407, 1246, 1048, 776, 753, 602,

MS (m/z): 468 (M⁺+1),

¹H NMR (DMSO-d₆, 400 MHz): δ 8.64 (d, J=1.6 Hz, 1H), 8.24 (t, J=5.7 Hz, 1H), 8.10 (d, J=1.3 Hz, 1H), 7.87 (t, J=8.7 Hz, 1H), 7.81 (dd, J=2.4 & 13.4 Hz, 1H), 7.54 (dd, J=1.8 & 8.8 Hz), 6.76 (d, J=2.4 Hz, 1H), 5.63 (s, 2H), 4.80-4.77 (m, 1H), 4.20 (t, J=9.0 Hz, 1H), 3.82 (dd, J=6.4 & 9.1 Hz, 1H), 3.45 (t, J=5.5 Hz, 2H), 1.84 (s, 3H).

### Example 33

### Methyl 3-[3-Fluoro-4-(4-imidazol-1-ylmethyl-thiazol-2-yl)-phenyl]-2-oxo-oxazolidin-5(S)-ylmethyl}-carbamate

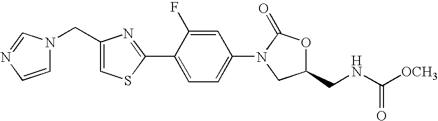

Tert-Butyl-{3-[3-fluoro-4-(4-imidazol-1-ylmethyl-thiazol-2-yl)-phenyl]-2-oxo-oxazolidin-5(S)-ylmethyl}-carbamate (0.6 grams, 1.26 mmol) and trifluoroacetic acid (3 mL) were stirred in dichloromethane (10 mL) for 1 hour at room temperature. Dichloromethane and trifluoroacetic acid were completely evaporated and diethyl ether was added to it. The solid thus obtained was dissolved in dichloromethane (10 mL) followed by the addition of triethyl amine (0.51 mL, 5.07 mmol) at 0° C. Methyl chloroformate (0.18 mL, 1.9 mmol) was added dropwise and allowed to stir at room temperature for 1 hour. Volatiles were evaporated and the residue obtained was purified by column chromatography over basic alumina using 2% methanol in chloroform as eluent to obtain the title compound

Yield: 12%,

Melting Range: 165-167° C.

MS (m/z): 432 (M⁺+1), 364,

¹H NMR (300 MHz, DMSO-d₆): δ 8.20 (t, J=8.8 Hz, 1H), 7.79 (s, 1H), 7.69 (dd, J=2.1 & 14.1 Hz, 1H), 7.60 (s, 1H), 7.56 (bs, 1H), 7.50 (dd, J=2.2 & 8.9 Hz, 1H), 7.27 (s, 1H), 6.92 (s, 1H), 5.38 (s, 2H), 4.82-4.72 (m, 1H), 4.18 (t, J=9.1 Hz, 1H), 3.83 (dd, J=6.4 & 9.2 Hz, 1H), 3.54 (s, 3H), 3.37 (t, J=5.7 Hz, 2H).

### Example 34

### Methyl {3-[3-fluoro-4-(4-pyrazol-1-ylmethyl-oxazol-2-yl)-phenyl]-2-oxo-oxazolidin-5(S)-ylmethyl}-carbamate

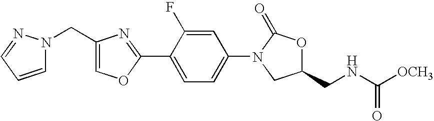

N-{3-[3-Fluoro-4-(4-pyrazol-1-ylmethyl-oxazol-2-yl)-phenyl]-2-oxo-oxazolidin-5(S)-ylmethyl}-acetamide was hydrolyzed with 6N hydrochloric acid to the corresponding amine, which was then converted to the title compound using methyl chloroformate.

MS (m/z): 416 (M⁺+1),

Melting Range: 170-172° C.,

¹H NMR (400 MHz, CDCl₃): δ 8.01 (t, J=8.5 Hz, 1H), 7.55-7.62 (m, 3H), 7.32 (dd, J=2.2 & 8.6 Hz, 2H), 6.29 (t, J=1.9 Hz, 1H), 5.34 (d, J=0.8 Hz, 2H), 5.14 (bs, 1H), 4.77-4.83 (m, 1H), 4.10 (t, J=9.0, 1H); 3.86 (t, J=7.9 Hz, 1H), 3.68 (s, 3H), 3.54-3.65 (m, 2H).

### Example 35

### Methyl {3-[3-fluoro-4-(4-[1,2,4]triazol-1-ylmethyl-thiazol-2-yl)-phenyl]-2-oxo-oxazolidin-5(S)-ylmethyl}-carbamate

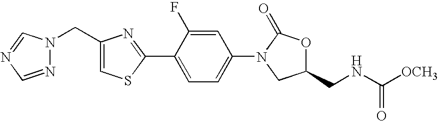

The title compound is prepared by following the procedure as described in example 33, by taking appropriate starting materials.

Yield: 22%,

Melting Range: 199-201° C.,

MS (m/z): 433 (M⁺+1), 364,

¹H NMR (300 MHz, CDCl₃): δ 8.30 (s, 1H), 8.24 (t, J=8.7 Hz, 1H), 7.99 (s, 1H), 7.69 (dd, J=2.1 & 13.4 Hz, 1H), 7.29-7.24 (m, 2H), 5.55 (s, 2H), 5.16 (bs, 1H), 4.82-4.81 (m, 1H), 4.12 (t, J=9.0 Hz, 1H), 3.88 (t, J=7.8 Hz, 1H), 3.69 (s, 3H), 3.66-3.59 (m, 2H).

### Example 36

### Methyl {3-[3-fluoro-4-(4-pyrazol-1-ylmethyl-thiazol-2-yl)-phenyl]-2-oxo-oxazolidin-5(S)-ylmethyl}-carbamate

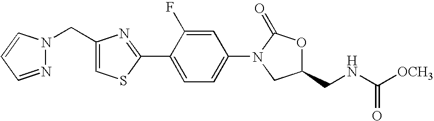

The title compound is prepared by following the procedure as described in example 33, by taking appropriate starting materials.

Yield: 12%,

Melting Range: 142-144° C.,

MS (m/z): 432 (M⁺+1), 364,

¹H NMR (300 MHz, DMSO-d₆): δ 8.19 (t, J=8.8 Hz, 1H), 7.87 (d, J=2.1 Hz, 1H), 7.69 (dd, J=2.0 & 14.2 Hz, 1H), 7.56 (bs, 1H), 7.53-7.46 (m, 3H), 6.30 (t, J=2.0 Hz, 1H), 5.51 (s, 2H), 4.81-4.72 (m, 1H), 4.18 (t, J=9.1 Hz, 1H), 3.83 (dd, J=6.5 & 9.5 Hz, 1H), 3.54 (s, 3H), 3.37 (t, J=5.2 Hz, 2H).

### Example 37

### 5-Aminomethyl-3-[3-fluoro-4-(2-pyrazol-1-ylmethyl-thiazol-4-yl)-phenyl]-oxazolidin-2-one

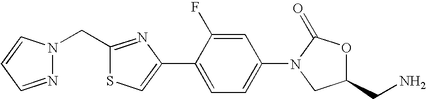

A solution of N-{3-[3-fluoro-4-(2-pyrazol-1-ylmethyl-thiazol-4-yl)-phenyl]-2-oxo-oxazolidin-5-ylmethyl}-acetamide (280 mg, 0.73 mmol) in 2N hydrochloric acid (10 mL) was refluxed for 2 hours. Ice-water was added and reaction mixture was neutralized with solid sodium bicarbonate at 0° C. The reaction mixture was extracted with chloroform (2×100 mL) and the chloroform layer was then washed with water and brine solution. Organic portion was then dried over sodium sulphate and concentrated to obtain a cream colour solid

Yield: 77%,

IR (KBr, cm⁻¹): 3416, 1727, 1627, 1410, 1232, 1193, 1028 and 751,

MS (m/z): 374 (M⁺+1),

¹H NMR (200 MHz, CDCl₃): δ 8.16 (t, J=8.6 Hz, 1H), 7.64-7.59 (m, 3H), 7.31-7.24 (m, 2H), 6.32 (s, 1H), 5.66 (s, 2H), 4.70 (bs, 1H), 4.07-3.90 (m, 2H), 3.11-3.00 (m, 2H), 2.20 (bs, 2H).

### Example 38

### Methyl {3-[3-fluoro-4-(2-pyrazol-1-ylmethyl-thiazol-4-yl}-phenyl]-2-oxo-oxazolidin-5-ylmethyl}-carbamate

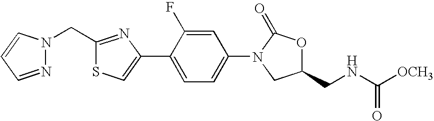

To a solution of 5-aminomethyl-3-[3-fluoro-4-(2-pyrazol-1-ylmethyl-thiazol-4-yl)-phenyl]-oxazolidin-2-one (40 mg, 0.11 mmol) in dry dichloromethane (2 mL) was added triethyl amine (0.03 mL, 0.23 mmol) followed by the addition of methyl chloroformate (0.1 mL, 0.14 mmol) at 0° C. The reaction mixture was stirred for 0.5 hours at 0° C. and then diluted with dichloromethane (60 mL). Dichloromethane layer was washed with brine solution, dried over sodium sulphate and concentrated. The residue obtained was purified by column chromatography over silica gel (100-200 mesh) using chloroform:methanol (24:1) as eluent. Title compound was obtained as white solid

Yield: 61%,

IR (KBr, cm⁻¹): 3361, 2959, 1772, 1542, 1405, 1195, 1145, 865, 752 and 636,

MS (m/z): 432 (M⁺+1), 400, 374,

¹H NMR (400 MHz, CDCl₃): δ 8.20 (t, J=8.6 Hz, 1H), 7.67-7.59 (m, 4H), 7.27-7.23 (m, 2H), 6.35 (t, J=2.2 Hz, 1H), 5.69 (s, 2H), 5.12 (bs, 1H), 4.81-4.78 (m, 1H), 4.12-4.08 (m, 1H), 3.87-3.83 (m, 1H), 3.69 (s, 3H), 3.65-3.55 (m, 2H).

### Example 39

### Methyl {3-[3-fluoro-4-(3-imidazol-1-ylmethyl-pyrazol-1-yl)phenyl]-2-oxo-oxazolidin-5-(S)-ylmethyl}-carbamate

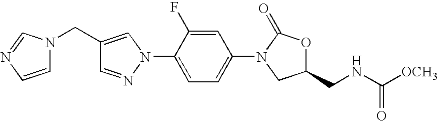

To a solution of 5-(R)-azidomethyl-3-[3-fluoro-4-(4-imidazol-1-ylmethyl-pyrazol-1-yl)-phenyl]-oxazolidin-2-one (0.17 grams, 0.44 mmol) in tetrahydrofuran (15 mL) was added triphenylphosphene (0.23 grams, 0.89 mmol) in small batches at room temperature and stirred for 2 hours. Water (0.7 mL) was added and heated to 75° C. While stirring for 4 hours. Solvent was removed by azeotropic distillation with benzene and the residue obtained was dissolved in dichloromethane (10 mL). Triethyl amine (0.19 mL) was added followed by the addition of methylchloroformate (0.04 mL, 0.58 mmol) at ice temperature. Stirring was continued for one hour at the same temperature. Solvent was removed and few pieces of ice were added. The solid obtained was dissolved in chloroform and it was washed with brine. Finally it was dried over anhydrous sodium sulfate and the volatiles were evaporated. The residue was purified by column chromatography (basic alumina) with chloroform and methanol mixture. Product was collected at 0.3% methanol in chloroform.

Yield: 36%,

Melting Range: 196-198° C.,

MS (m/z): 415 (M⁺+1),

¹H NMR (300 MHz, CDCl₃): δ 7.93 (d, J=2.3 Hz, 1H), 7.87 (t, J=8.9 Hz, 1H), 7.75 (dd, J=2.4 & 13.9 Hz, 1H), 7.61-7.67 (m, 2H), 7.21-7.41 (m, 1H), 7.10 (s, 1H), 6.98 (s, 1H), 5.11 (s, 2H), 4.80-4.82 (m, 1H), 4.10 (t, J=9.0 Hz, 1H), 3.86 (t, J=7.2 Hz, 1H), 3.69 (s, 3H), 3.55-3.65 (m, 2H).

### Example 40

### Methyl {3-[3-fluoro-4-(3-pyrazol-1-ylmethyl-pyrazol-1-yl)phenyl]-2-oxo-oxazolidin-5-(S)-ylmethyl}-carbamate

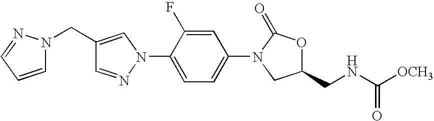

Title compound was obtained from 5-(R)-azidomethyl-3-[3-fluoro-4-(4-pyrazol-1-ylmethyl-pyrazol-1-yl)-phenyl]-oxazolidin-2-one (0.42 grams, 1.09 mmol) following the procedure reported for {3-[3-fluoro-4-(3-imidazol-1-ylmethyl-pyrazol-1-yl)phenyl]-2-oxo-oxazolidin-5-(S)-ylmethyl}-carbamic acid methyl ester.

Yield: 72%,

Melting Range: 156-158° C.,

MS (m/z): 415 (M⁺+1),

¹H NMR (300 MHz, CDCl₃): δ 7.98 (d, J=2.3 Hz, 1H), 7.86 (t, J=8.8 Hz, 1H), 7.68-7.78 (m, 2H), 7.55 (s, 1H), 7.43 (d, J=2.1 Hz, 1H), 7.18-7.25 (m, 1H), 6.28 (t, J=1.9 Hz, 1H), 5.31 (s, 2H), 5.15 (bs, 1H), 4.75-4.85 (m, 1H), 4.09 (t, J=9.0 Hz, 1H), 3.85 (t, J=7.3 Hz, 1H), 3.68 (s, 3H), 3.52-3.66 (m, 2H).

### Example 41

### Methyl {3-[3-fluoro-4-(3-[1,2,4]-triazol-1-ylmethyl-pyrazol-1-yl)phenyl]-2-oxo-oxazolidin-5-(S)-ylmethyl}-carbamate

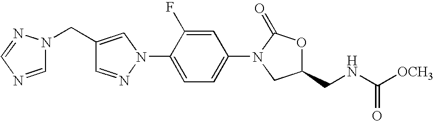

Title compound was obtained from 5-(R)-azidomethyl-3-[3-fluoro-4-(4-[1,2,4]triazol-1-ylmethyl-pyrazol-1-yl)-phenyl]-oxazolidin-2-one (0.5 grams, 1.31 mmol) following the procedure reported for methyl {3-[3-fluoro-4-(3-[1,2,4]triazol-1-ylmethyl-pyrazol-1-yl)phenyl]-2-oxo-oxazolidin-5-(S)-ylmethyl}-carbamate.

Yield: 59%,

Melting Range: 128-130° C.,

MS (m/z): 415 (M⁺+1),

¹H NMR (300 MHz, CDCl₃): δ 8.11 (s, 1H), 8.05 (d, J=2.5 Hz, 1H), 7.99 (s, 1H), 7.87 (t, J=8.9 Hz, 1H), 7.72-7.78 (m, 2H), 7.40 (s, 1H), 7.24-7.27 (m, 1H), 7.20-7.23 (m, 1H), 5.14 (s, 2H), 5.16 (t, J=5.8 Hz, 1H), 4.76-4.86 (m, 1H), 4.10 (t, J=9.0 Hz, 1H), 3.86 (dd, J=1.8 & 7.0 Hz, 1H), 3.69 (s, 3H), 3.52-3.65 (m, 2H).

1. J. Org. Chem. 1982, 47, 2216-2217.

### Example 42

### 5-Aminomethyl-3-[3-fluoro-4-(4-imidazol-1-ylmethyl-imidazol-1-yl)-phenyl]-oxazolidin-2-one

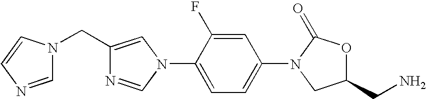

To a solution of 5-azidomethyl-3-[3-fluoro-4-(4-imidazol-1-ylmethyl-imidazol-1-yl)-phenyl]-oxazolidin-2-one (1 grams, 2.6 mmol) in tetrahydrofuran (10 mL) was added triphenyl phosphine (0.9 grams, 3.33 mmol) in batches at room temperature and stirred for 5 hours. Water (1 mL) was added and the resulting mixture was heated to 60° C. for 3 hours. Tetrahydrofuran was evaporated under reduced pressure and water was evaporated by azeotropic distillation with benzene (4×10 mL). The residue obtained was purified by column chromatography on silica gel (100-200 mesh) using a mixture of methanol and chloroform (1:9) to obtain the title compound.

Yield: 100%.

IR (KBr, cm⁻¹): 3416, 1746, 1533, 1410, 1218, 1137, 871, 813, 746, 682,

MS (m/z): 359 (M⁺+1), 330, 261, 189, 175,

¹H NMR (DMSO-d₆, 400 MHz): δ 8.67 (s, 1H), 8.60 (s, 1H), 7.99 (s, 1H), 7.87-7.80 (m, 2H), 7.54 (dd, J=1.3 & 8.8 Hz, 1H), 5.62 (s, 2H), 4.73-4.66 (m, 1H), 4.15 (t, J=8.8 Hz, 1H), 3.94 (dd, J=6.2 & 8.9 Hz, 1H), 2.93-2.82 (m, 2H).

### Example 43

### Methyl {3-[3-fluoro-4-(4-imidazol-1-ylmethyl-imidazol-1-yl)-phenyl]-2-oxo-oxazolidin-5-ylmethyl}-carbamate

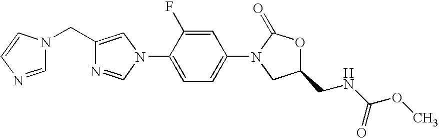

To a solution of 5-aminomethyl-3-[3-fluoro-4-(4-imidazol-1-ylmethyl-imidazol-1-yl)-phenyl]-oxazolidin-2-one (0.57 grams, 1.57 mmol) in dichloromethane (10 mL) was added triethyl amine (0.9 mL, 6.3 mmol) followed by the drop wise addition of methyl chloroformate (0.25 mL, 3.2 mmol) at 5-10° C. Stirring was continued at room temperature for 1 hour and then water (25 mL) was added to the reaction mixture. The aqueous layer was extracted with dichloromethane (3×25 mL). Combined extract was dried over sodium sulfate and concentrated to obtain the title compound.

Yield: 15%,

Melting Range: 78-80° C.,

MS (m/z): 415 (M⁺+1), 347,

¹H NMR (300 MHz, CDCl₃): δ 7.62-7.74 (m, 3H) 7.28-7.38 (m, 3H), 7.06 (s, 2H), 5.60 (t, 1H), 5.14 (s, 2H), 4.76-4.84 (m, 1H), 4.06 (t, J=8.9 Hz, 1H), 3.86 (t, J=7.8 Hz, 1H), 3.68 (s, 3H), 3.56-3.64 (m, 2H).

### Example 44

### Methyl {3-[3-fluoro-4-(4-pyrazol-1-ylmethyl-imidazol-1-yl)-phenyl]-2-oxo-oxazolidin-5-ylmethyl}-carbamate

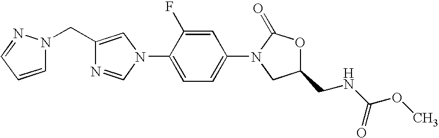

The title compound is prepared by following the procedure as described in example 43, by taking appropriate starting materials.

Yield: 15%,

Melting Point: 130° C.,

MS (m/z): 415 (M⁺+1), 347,

¹H NMR (300 MHz, CDCl₃): δ 7.54-7.76 (m, 3H) 7.28-7.39 (m, 2H), 7.16 (s, 1H), 6.30 (t, J=2.0 Hz, 1H), 5.35 (s, 2H), 5.26 (t, 1H), 4.78-4.86 (m, 1H), 4.10 (t, J=8.9 Hz, 1H), 3.86 (t, J=7.8 Hz, 1H), 3.70 (s, 3H), 3.66-3.58 (m, 2H).

### Example 45

### Methyl {3-[3-fluoro-4-(4-[1,2,4]-triazol-1-ylmethyl-imidazol-1-yl)-phenyl]-2-oxo-oxazolidin-5-ylmethyl}-carbamate

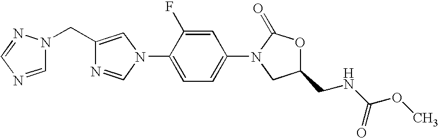

The title compound is prepared by following the procedure as described in example 43, by taking appropriate starting materials.

Yield: 46.6%,

Melting Range: 184-186° C.,

MS (m/z): 416 (M⁺+1), 347, 303,

¹H NMR (300 MHz, CDCl₃): δ 8.26 (s, 1H), 7.96 (s, 1H), 7.68-7.76 (m, 2H), 7.26-7.37 (m, 3H), 5.37 (s, 2H), 5.19 (bs, 1H), 4.80-4.95 (m, 1H), 4.09 (t, J=8.9 Hz, 1H), 3.87 (t, J=8.0 Hz, 1H), 3.60-3.75 (m, 5H).

### Example 46

### Methyl N-{3-[3-fluoro-4-(4-pyrazol-1-yl-methyl-(1,2,3)triazol-1-yl)-phenyl]-2-oxo-oxazolidin-5-ylmethyl}carbamate

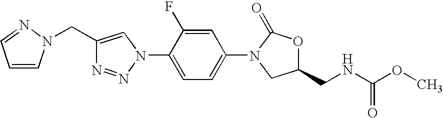

The title compound is prepared by following the procedure as described in example 43, by taking appropriate starting materials.

Yield: 75%,

IR (KBr, cm⁻¹): 3432, 3318, 3369, 2921, 1743, 1697, 1531, 1458,

MS (m/z): 416 (M⁺+1), 384, 160,

¹H NMR (DMSO-d₆, 400 MHz): δ 8.52 (s, 1H), 7.87-7.74 (m, 3H), 7.54-7.46 (m, 3H), 6.27 (t, J=2.0 Hz, 1H), 5.10 (s, 2H), 4.79-4.76 (m, 1H), 4.20 (t, J=2.0 Hz, 1H), 3.85 (dd, J=6.4 & 9.4 Hz, 1H), 3.54 (s, 3H), 3.38 (t, J=5.0 Hz, 2H).

### Example 47

### Ethyl 3-amino-1-(1-{2-fluoro-4-[5-(methoxycarbonylamino-methyl)-2-oxo-oxazolidin-3-yl]-phenyl}-1H-[1,2,3]triazol-4-ylmethyl)-1H-pyrazole-4-carboxylate

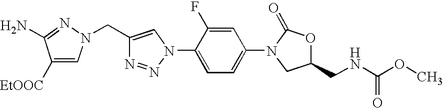

The title compound is prepared by following the procedure as described in example 28, by taking appropriate starting materials.

Yield: 21%,

Melting Range: 98-100° C.,

IR (KBr, cm⁻¹): 3443, 2927, 1756, 1609, 1527, 1408,

MS (m/z): 504 (M⁺+2), 503 (M⁺+1), 475, 410,

¹H NMR (DMSO-d₆, 400 MHz): δ 8.55 (s, 1H), 8.10 (bs, 1H), 7.87 (t, J=8.7 Hz, 1H), 7.80 (dd, J=2.2 & 13.4 Hz, 1H), 7.54 (dd, J=1.1 & 8.1 Hz, 2H), 5.35 (bs, 2H), 5.28 (s, 2H), 4.80-4.75 (m, 1H), 4.18-4.15 (m, 3H), 3.85 (dd, J=6.2 & 9.1 Hz, 1H), 3.55 (s, 3H), 3.39 (d, J=7.0 Hz, 2H), 1.25 (t, J=6.9 Hz, 3H).

### Example 48

### Methyl N-{3-[3-fluoro-4-(4-[1,2,4]-triazol-1-ylmethyl)-[1,2,3]triazol-1-yl)-phenyl]-2-oxo-oxazolidin-5-ylmethyl}-carbamate

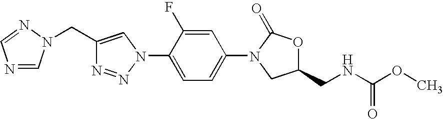

The title compound is prepared by following the procedure as described in example 28, by taking appropriate starting materials.

Yield: 54%,

Melting Point: 170° C.,

IR (KBr, cm⁻¹): 3420, 2938, 1734, 1632, 1530, 1413, 1289, 1241, 1135, 1052, 1016,

MS (m/z): 417 (M⁺+1), 391, 360, 298,

¹H NMR (DMSO-d₆, 400 MHz): δ 8.67 (s, 1H), 8.60 (d, J=1.6 Hz, 1H), 8.00 (s, 1H), 7.85 (t, J=8.9 Hz, 1H), 7.80 (dd, J=2.4 & 13.4 Hz, 1H), 7.53 (dd, J=1.3 & 8.0 Hz, 2H), 5.62 (s, 2H), 4.81-4.75 (m, 1H), 4.20 (t, J=9.0 Hz, 1H), 3.85 (dd, J=8.8 & 9.1 Hz, 1H), 3.55 (s, 3H), 3.38 (t, J=5.1 Hz, 2H).

### Example 49

### Methyl N-{3-[3-Fluoro-4-[4-(3-trifluoromethyl-pyrazol-1-yl-methyl-[1,2,3]triazol-1-yl)-phenyl]-2-oxo-oxazolidin-5-ylmethyl}-carbamate

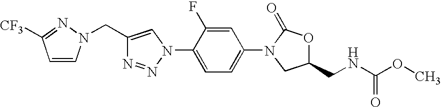

The title compound is prepared by following the procedure as described in example 28, by taking appropriate starting materials.

Yield: 36%,

Melting Range: 129-130° C.,

IR (KBr, cm⁻¹): 3348, 1734, 1531, 1286, 1244, 1128, 1057, 987,

MS (m/z): 484 (M⁺+1),

¹H NMR (DMSO-d₆, 400 MHz): δ 8.64 (d, J=1.9 Hz, 1H), 8.11-8.10 (m, 1H), 7.87 (t, J=8.6 Hz, 1H), 7.8 (dd, J=2.4 & 13.4 Hz, 1H), 7.53 (dd, J=1.6 & 6.4 Hz, 1H), 6.76 (d, J=2.4 Hz, 1H), 5.63 (s, 2H), 4.80-4.77 (m, 1H), 4.20 (t, J=9.1 Hz, 1H), 3.85 (dd, J=6.1 & 9.1 Hz, 1H), 3.55 (s, 3H), 3.39 (t, J=5.6 Hz, 2H).

### Example 50

### O-methyl {3-[3-fluoro-4-(4-pyrazol-1-ylmethyl-oxazol-2-yl)-phenyl]-2-oxo-oxazolidin-5(S)-ylmethyl}-thiocarbamate

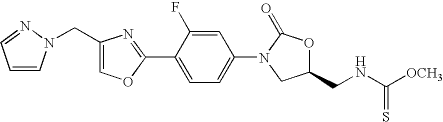

The amine obtained from N-{3-[3-fluoro-4-(4-pyrazol-1-ylmethyl-oxazol-2-yl)-phenyl]-2-oxo-oxazolidin-5(S)-ylmethyl}-acetamide was converted to isothiocyanate derivative in the usual way, which on refluxing with methanol gave the title compound.

MS (m/z): 432 (M⁺+1), 279, 177, 149,

Melting Range: 145-147° C.

¹H NMR (400 MHz, DMSO-d₆): δ 9.51 & 9.40 (2t, J=5.6 Hz, 1H, rotamers in 4:1 ratio), 8.16 (s, 1H), 7.99 (t, J=8.6 Hz, 1H), 7.81 (t, J=2.4 Hz, 1H), 7.65 (dd, J=1.9 & 13.7 Hz, 1H), 7.45-7.51 (m, 2H), 6.27 (t, J=2.0 Hz, 1H), 5.32 (s, 2H), 4.88-4.98 & 4.75-4.85 (2m, 1H, rotamers in a ratio of 4:1), 4.16-4.25 (m, 1H), 3.78-3.98 (m, 5H).

### Example 51

### O-methyl {3-[3-fluoro-4-(4-imidazol-1-ylmethyl-thiazol-2-yl)-phenyl]-2-oxo-oxazolidin-5(S)-ylmethyl}-thiocarbamate

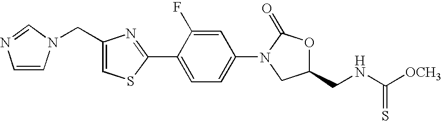

Tert-Butyl-{3-[3-fluoro-4-(4-imidazol-1-ylmethyl-thiazol-2-yl)-phenyl]-2-oxo-oxazolidin-5(S)-ylmethyl}-carbamate (1.0 gram, 2.1 mmol) and trifluoroacetic acid (3 mL) were stirred in dichloromethane (20 mL) for 1 hour at room temperature. Dichloromethane and trifluoroacetic acid were completely evaporated and diethyl ether was added to it. The solid thus obtained was dissolved in dichloromethane (10 mL) followed by the addition of triethylamine (0.51 mL, 5.07 mmol) at 0° C. Thiophosgene (0.36 mL, 3.16 mmol) was added dropwise and allowed to stir at room temperature for 0.5 hour. Volatiles were evaporated and the residue obtained was dissolved in methanol (25 mL). Reaction mixture was heated to 80° C. for 5 hours and then solvent evaporated. The residue was purified by column chromatography over basic alumina using 1% methanol in chloroform as eluent to obtain the title compound.

Yield: 12%,

Melting Range: 169-171° C.,

MS (m/z): 448 (M⁺+1), 432, 380,

¹H NMR (300 MHz, DMSO-d₆): δ 9.54 & 9.46 (2t, J=5.5 Hz, 1H, rotamers in 4:1 ratio), 8.18 (t, J=8.7 Hz, 1H), 7.78 (s, 1H), 7.67 (dd, J=2.1 & 14.2 Hz, 1H), 7.58 (s, 1H), 7.49 (dd, J=2.1 & 8.8 Hz, 1H), 7.25 (s, 1H), 6.91 (s, 1H), 5.36 (s, 2H), 4.98-4.89 (2m, 1H, rotamers in 4:1 ratio), 4.20 (t, J=9.0 Hz, 1H), 3.91 & 3.86 (2s, 3H, rotamers in 1:4 ratio), 3.88 (t, J=7.8 Hz, 1H), 3.78 (t, J=5.6 Hz, 2H).

### Example 52

### O-methyl {3-[3-fluoro-4-(4-[1,2,4]triazol-1-ylmethyl-thiazol-2-yl)-phenyl]-2-oxo-oxazolidin-5(S)-ylmethyl}-thiocarbamate

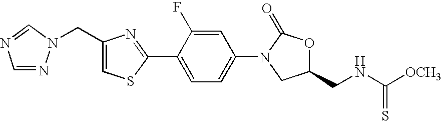

The title compound is prepared by following the procedure as described in example 51, by taking appropriate starting materials.

Yield: 32%,

Melting Range: 189-191° C.,

MS (m/z): 449 (M⁺+1), 380, 322,

¹H NMR (300 MHz, DMSO-d₆): δ 9.57 & 9.48 (2t, J=5.6 Hz, 1H, rotamers in 4:1 ratio), 8.70 (s, 1H), 8.16 (t, J=8.8 Hz, 1H), 8.01 (s, 1H), 7.69 (dd, J=2.2 & 14.2 Hz, 1H), 7.69 (s, 1H), 7.51 (dd, J=2.2 & 8.8 Hz, 1H), 5.61 (s, 2H), 4.97-4.92 & 4.88-4.79 (2m, 1H, rotamers in 4:1 ratio), 4.21 (t, J=8.9 Hz, 1H), 3.93 & 3.88 (2s, 3H rotamers in 1:4 ratio), 3.88 (t, J=7.8 Hz, 1H), 3.79 (t, J=5.6 Hz, 2H).

### Example 53

### O-methyl {3-[3-fluoro-4-(4-pyrazol-1-ylmethyl-thiazol-2-yl)-phenyl]-2-oxo-oxazolidin-5(S)-ylmethyl}-thiocarbamate

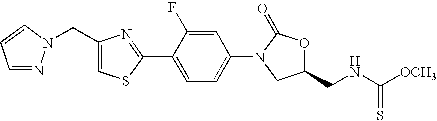

The title compound is prepared by following the procedure as described in example 51, by taking appropriate starting materials.

Yield: 14%,

Melting Range: 157-158° C.,

MS (m/z): 448 (M⁺+1), 380, 336,

¹H NMR (300 MHz, DMSO-d₆): δ 9.56 & 9.48 (2t, J=5.7 Hz, 1H, rotamers in 4:1 ratio), 8.18 (t, J=8.8 Hz, 1H), 7.87 (dd, J=0.6 & 2.2 Hz, 1H), 7.68 (dd, J=2.1 & 14.2 Hz, 1H), 7.53-7.46 (m, 3H), 6.30 (t, J=2.1 Hz, 1H), 5.51 (s, 2H), 4.98-4.90 & 4.86-4.78 (2m, 1H, rotamers in 4:1 ratio), 4.21 (t, J=9.0 Hz, 1H), 3.93 & 3.88 (2s, 3H rotamers in 1:4 ratio), 3.89 (t, J=7.9 Hz, 1H), 3.79 (t, J=5.6 Hz, 2H).

### Example 54

### O-Methyl {3-[3-fluoro-4-(2-pyrazol-1-ylmethyl-thiazol-4-yl)-phenyl]-2-oxo-oxazolidin-5-ylmethyl}-thiocarbamate

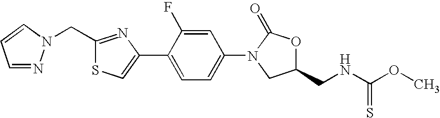

To a solution of 5-aminomethyl-3-[3-fluoro-4-(2-pyrazol-1-ylmethyl-thiazol-4-yl)-phenyl]-oxazolidin-2-one (40 mg, 0.11 mmol) in a mixture of water and chloroform (1:1, 2 mL) was added sodium bicarbonate (106 mg, 1.26 mmol) followed by the addition of thiophosgene (0.2 mL, 0.29 mmol) at 0° C. and stirred for 2 hours at 0° C. The reaction mixture was diluted with ethyl acetate (50 mL) and the ethyl acetate layer was then washed with brine solution and dried over sodium sulphate. The organic layer was evaporated and the residue was refluxed in methanol for 12 hours. Evaporation of methanol left a residue, which was purified by column chromatography over silica gel (100-200 mesh) using chloroform and methanol (24:1) as eluent to obtain a white solid.

Yield: 28%,

IR (KBr, cm⁻¹): 3250, 2941, 2360, 1764 and 1409,

MS (m/z): 404 (M⁺-44), 372,

¹H NMR (200 MHz, CDCl₃): δ 8.18 (t, J=8.6 Hz, 1H), 7.66-7.55 (m, 4H), 7.24-7.19 (m, 1H), 6.36-6.34 (m, 1H), 5.69 (s, 2H), 4.97-4.94 (m, 1H), 4.16-4.08 (m, 4H), 4.01 (s, 3H).

### Example 55

### Methyl {3-[3-fluoro-4-(4-pyrazol-1-ylmethyl-imidazol-1-yl)-phenyl]-2-oxo-oxazolidin-5-ylmethyl}-thiocarbamate

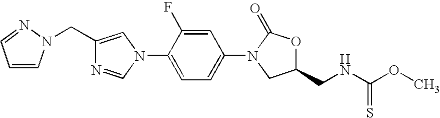

A solution of 3-[3-fluoro-4-(4-pyrazol-1-ylmethyl-imidazol-1-yl)-phenyl]-5-isothiocyanatomethyl-oxazolidin-2-one (0.185 grams, 0.05 mmol) in methanol (10 mL) was heated to 55° C. for 18 hours. Solvent was evaporated and the residue was purified by column chromatography on silica gel (100-200 mesh) using a mixture of methanol and chloroform (1:9) to obtain the title compound.

Yield: 78.3%,

Melting Range: 116-188° C.,

MS (m/z): 431 (M⁺+1), 363, 319,

¹H NMR (300 MHz, CDCl₃): δ 7.54-7.76 (m, 4H), 7.28-7.40 (m, 3H), 7.16 (s, 1H), 6.74 (bs, 1H), 6.28 (s, 1H), 5.35 (s, 2H), 5.02-4.92 (bs, 1H), 3.90-4.16 (m, 4H),

### Example 56

### Methyl {3-[3-Fluoro-4-(4-pyrazol-1-yl-methyl-[1,2,3]triazol-1-yl)-phenyl]-2-oxo-oxazolidin-5-yl-methyl}-thiocarbamate

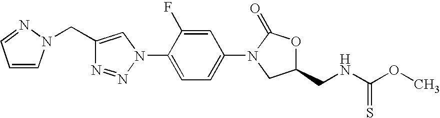

The title compound is prepared by following the procedure as described in example 54, by taking appropriate starting materials.

Yield: 37%,

Melting Range: 180-182° C.,

IR (KBr, cm⁻¹): 3445, 2925, 1754, 1530, 1400, 1229, 1047, 755,

MS (m/z): 400 (M⁺-31), 388,

¹H NMR (DMSO-d₆, 400 MHz): δ 9.52 (t, J=5.6 Hz, 1H); 8.52 (s, 1H); 7.87-7.77 (m, 3H); 7.54 (dd, J=1.9 & 10.0 Hz, 2H); 7.46 (dd, J=0.5 & 1.0 Hz, 1H); 6.20 (t, J=2.1 Hz, 1H); 4.97-4.94 (m, 1H); 4.23 (t, J=9.1 Hz, 1H); 3.94-3.72 (m, 5H).

### Example 57

### Methyl N-{3-[3-fluoro-4-(4-[1,2,4]-triazol-1-ylmethyl)-[1,2,3]triazol-1-yl)-phenyl]-2-oxo-oxazolidin-5-ylmethyl}-thiocarbamate

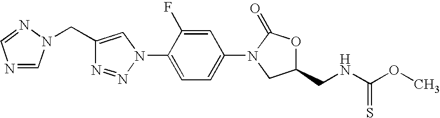

The title compound is prepared by following the procedure as described in example 54, by taking appropriate starting materials.

Yield: 15%,

Melting Range: 172-173° C.

IR (KBr, cm⁻¹): 3265, 2925, 1755, 1528, 1406, 1210, 1140, 864, 748, 678,

MS (m/z): 400 (M⁺-32), 388,

¹H NMR (DMSO-d₆, 400 MHz): δ 9.53 & 9.44 (2t, J=5.8 Hz, 1H, rotamers in 4:1 ratio), 8.67 (s, 1H); 8.60 (d, J=1.9 Hz, 1H) 7.99 (s, 1H), 7.86 (t, J=8.9 Hz, 1H), 7.80 (dd, J=2.4 & 13.4 Hz, 1H), 7.54 (dd, J=1.6 & 8.9 Hz, 1H), 5.62 (s, 2H), 4.98-4.94 (m, 1H, rotamers in 4:1 ratio), 4.23 (t, J=9.1 Hz, 1H), 3.94 & 3.89 (2s, 3H, rotamers in 1:4 ratio), 3.93-3.90 (m, 1H), 3.81 (t, J=5.6 Hz, 2H).

### Example 58

### 5-Aminomethyl-3-{3-fluoro-4-[4-(3-trifluoromethyl-pyrazol-1-ylmethyl-[1,2,3]triazol-1-yl)-phenyl]-oxazolidin-2-one

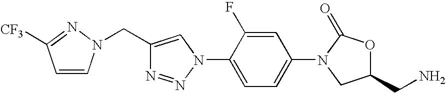

A solution of N-{3-[3-fluoro-4-[4-(3-trifluoromethyl-pyrazol-1-ylmethyl-[1,2,3]triazol-1-yl]-phenyl}-2-oxo-oxazolidin-5-ylmethyl}-acetamide (300 mg, 0.63 mmol) and 4N hydrochloric acid (2.5 mL) in methanol (2.5 mL) was refluxed for 10 hours. The pH of the reaction mixture was adjusted to 12 by the addition of saturated solution of sodium carbonate. It was then extracted with chloroform (40 mL×2) and the combined extract was washed with brine. Finally, it was dried over sodium sulfate and volatiles were evaporated. The residue obtained was purified by column chromatography on silica gel using methanol and chloroform (1:9) as eluent to obtain the title compound as white solid.

Yield: 70%,

IR (KBr, cm⁻¹): 3427, 1741, 1530, 1412, 1243, 1129, 1050, 968, 772,

MS (m/z): 426 (M⁺+1),

¹H NMR (DMSO-d₆, 400 MHz): δ 8.64 (d, J=1.9 Hz, 1H), 8.11 (d, J=1.3 Hz, 1H), 7.87 (t, J=8.6 Hz, 1H), 7.82 (dd, J=2.4 & 13.4 Hz, 1H), 7.56 (dd, J=1.6 & 8.0 Hz, 1H), 6.75 (d, J=2.1 Hz, 1H), 5.63 (s, 2H), 4.78-4.74 (m, 1H), 4.20 (t, J=9.0 Hz, 1H), 4.40 (bs, 2H), 3.94 (dd, J=6.4 & 9.1 Hz, 2H), 3.03-2.93 (m, 2H).

### Example 59

### Methyl {3-[3-fluoro-4-[4-(3-trifluoromethyl-pyrazol-1-yl-methyl-[1,2,3]triazol-1-yl)-phenyl]-2-oxo-oxazolidin-5-yl-methyl}-thiocarbamate

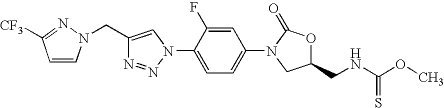

The title compound is prepared by following the procedure as described in example 54, by taking appropriate starting materials.

Yield: 42%,

Melting Range: 179-180° C.,

IR (KBr, cm⁻¹): 3424, 2926, 1751, 1528, 1410, 1242, 1051, 816, 769,

MS (m/z): 500 (M⁺+1), 275, 200, 131,

¹H NMR (DMSO-d₆, 400 MHz): δ 9.53 & 9.45 (2t, J=5.6 Hz, 1H, rotamers in 4:1 ratio), 8.64 (d, J=1.6 Hz, 1H), 8.11 (d, J=1.3 Hz, 1H), 7.87 (t, J=8.9 Hz, 1H), 7.81 (dd, J=2.2 & 13.4 Hz, 1H), 7.54 (dd, J=1.6 & 8.8 Hz, 1H), 6.76 (d, J=2.1 Hz, 1H), 5.64 (s, 2H), 5.00-4.83 (2m, 1H, rotamers in 4:1 ratio), 4.24 (t, J=9.1 Hz, 1H), 3.95 & 3.90 (2s, 3H, rotamers in 1:4 ratio), 3.93-3.91 (m, 1H), 3.81 (t, J=5.6 Hz, 2H).

### Example 60

### N-{3-[3-fluoro-4-(4-imidazol-1-ylmethyl-thiazol-2-yl)-phenyl]-2-oxo-oxazolidin-5(S)-ylmethyl}-thioacetamide

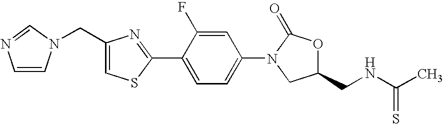

A mixture of N-{3-[3-fluoro-4-(4-imidazol-1-ylmethyl-thiazol-2-yl)-phenyl]-2-oxo-oxazolidin-5(S)-ylmethyl}-acetamide (0.36 grams, 0.87 mmol) and Lawesson's reagent (0.21 grams, 0.52 mmol) in dry dioxane was heated to 60° C. for 20 minutes. Dioxane was evaporated completely and the residue purified by column chromatography over basic alumina using 2% methanol in chloroform as eluent to obtain the title compound

Yield: 53.5%,

Melting Range: 169-171° C.,

MS (m/z): 432 (M⁺+1), 416, 388, 320,

¹H NMR (300 MHz, CDCl₃): δ 10.39 (t, J=5.0 Hz, 1H), 8.20 (t, J=8.8 Hz, 1H), 7.78 (s, 1H), 7.70 (dd, J=2.1 & 14.2 Hz, 1H), 7.60 (s, 1H), 7.52 (dd, J=2.1 & 8.8 Hz, 1H), 7.26 (s, 1H), 6.92 (s, 1H), 5.38 (s, 2H), 5.02-4.98 (m, 1H), 4.22 (t, J=9.1 Hz, 1H), 3.95-3.85 (m, 3H), 2.42 (s, 3H).

### Example 61

### N-{3-[3-fluoro-4-(4-[1,2,4]triazol-1-ylmethyl-thiazol-2-yl)-phenyl]-2-oxo-oxazolidin-5(S)-ylmethyl}-thioacetamide

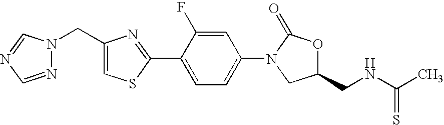

The title compound is prepared by following the procedure as described in example 60, by taking appropriate starting materials.

Yield: 34.4%.

Melting Range: 180-182° C.,

MS (m/z): 433 (M⁺+1), 389, 320,

¹H NMR (300 MHz, DMSO-d₆): δ 10.39 (t, J=5.2 Hz, 1H), 8.69 (s, 1H), 8.16 (t, J=8.8 Hz, 1H), 8.00 (s, 1H), 7.70 (dd, J=2.1 & 14.1 Hz, 1H), 7.69 (s, 1H), 7.51 (dd, J=2.1 & 8.8 Hz, 1H) 5.61 (s, 2H), 5.02-4.97 (m, 1H), 4.22 (t, J=9.1 Hz, 1H), 3.94-3.85 (m, 3H), 2.44 (s, 3H).

### Example 62

### N-{3-[3-fluoro-4-(4-pyrazole-1-ylmethyl-thiazol-2-yl)-phenyl]-2-oxo-oxazolidin-5(S)-ylmethyl}-thioacetamide

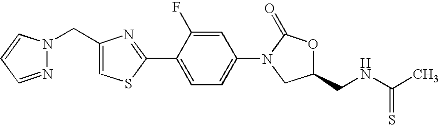

The title compound is prepared by following the procedure as described in example 60, by taking appropriate starting materials.

Yield: 24%

Melting Range: 166-168° C.

MS (m/z): 432 (M⁺+1), 388, 320,

¹H NMR (300 MHz, DMSO-d₆): δ 10.38 (bs, 1H), 8.19 (t, J=8.8 Hz, 1H), 7.87 (d, J=1.8 Hz, 1H), 7.70 (dd, J=2.2 & 14.2 Hz, 1H), 7.54-7.41 (m, 3H), 6.30 (t, J=2.1 Hz, 1H), 5.51 (s, 2H), 5.05-4.95 (m, 1H), 4.23 (t, J=9.0 Hz, 1H), 3.94-3.85 (m, 3H), 2.44 (s, 3H).

### Example 63

### N-{3-[3-Fluoro-4-(2[1,2,4]-triazol-1-ylmethyl-thiazol-4-yl)-phenyl]-2-oxo-oxazolidin-5-ylmethyl}-thioacetamide

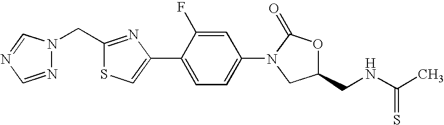

The title compound is prepared by following the procedure as described in example 60, by taking appropriate starting materials.

Melting Range: 82-84° C.

IR (KBr, cm⁻¹): 1752, 1406 and 1196,

MS (m/z): 431 (M⁺−1), 389,

¹H NMR (200 MHz, DMSO-d₆): δ 10.40 (bs, 1H), 8.79 (s, 1H), 8.20-8.01 (m, 2H), 7.93 (d, J=2.4 Hz, 1H), 7.60 (d, J=13.7 Hz, 1H), 7.45 (d, J=8.8 Hz, 1H), 5.90 (s, 2H), 5.02 (bs, 1H), 4.22 (t, J=8.8 Hz, 1H), 3.98-3.80 (m, 3H), 2.45 (s, 3H).

### Example 64

### N-{3-[3-Fluoro-4-(2-tetrazol-1-ylmethyl-thiazol-4-yl)-phenyl]-2-oxo-oxazolidin-5-ylmethyl}-thioacetamide

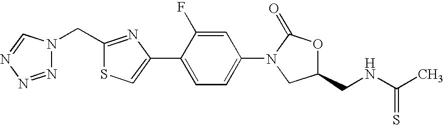

The title compound is prepared by following the procedure as described in example 60, by taking appropriate starting materials.

Melting Range: 96-98° C.,

IR (KBr, cm⁻¹): 1752, 1408 and 1226,

MS (m/z): 389 (Mt44), 356, 220, 219, 179, 148, 101,

¹H NMR (200 MHz, DMSO-d₆): δ 10.39 (bs, 1H), 9.66 (s, 1H), 8.08-8.01 (m, 2H), 7.65 (d, J=14.7 Hz, 1H), 7.43 (d, J=8.3 Hz, 1H), 6.23 (s, 2H), 4.99 (bs, 1H), 4.21 (t, J=8.8 Hz, 1H), 3.96-3.81 (m, 3H), 2.45 (s, 3H).

### Example 65

### N-{3-[3-fluoro-4-(4-imidazol-1-ylmethyl-imidazol-1-yl)-phenyl]-2-oxo-oxazolidin-5-ylmethyl}-thioacetamide

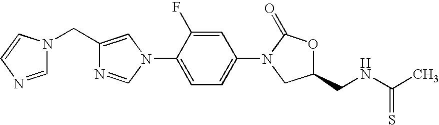

The title compound is prepared by following the procedure as described in example 60, by taking appropriate starting materials.

Yield: 48%,

Melting Range: 160-162° C.,

MS (m/z): 414 (M⁺+1), 371, 347, 303,

¹H NMR (300 MHz, CDCl₃): δ 7.60-7.73 (m, 3H), 7.23-7.37 (m, 2H), 7.00-7.10 (m, 3H), 5.10 (s, 2H), 4.90-5.05 (m, 1H), 4.19-4.20 (m, 3H), 3.89 (t, J=7.1 Hz, 1H), 2.54 (s, 3H).

### Example 66

### N-{3-[3-fluoro-4-(4-pyrazol-1-ylmethyl-imidazol-1-yl)-phenyl]-2-oxo-oxazolidin-5-ylmethyl}-thioacetamide

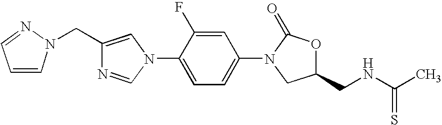

The title compound is prepared by following the procedure as described in example 60, by taking appropriate starting materials.

Yield: 17.6%,

Melting Range: 137-139° C.,

MS (m/z): 415 (M⁺+1), 347,

¹H NMR (300 MHz, CDCl₃): δ 8.20 (bs, 1H), 7.52-7.76 (m, 4H), 7.21-7.38 (m, 3H), 7.16 (s, 1H), 6.28 (t, J=2.1 Hz, 1H), 5.34 (s, 2H), 5.00-5.06 (m, 1H), 4.22-4.32 (m, 1H), 4.16-4.06 (m, 1H), 3.84-3.92 (m, 1H), 2.60 (s, 3H).

### Example 67

### N-{3-[3-fluoro-4-(4-[1,2,4]triazol-1-ylmethyl-imidazol-1-yl)-phenyl]-2-oxo-oxazolidin-5-ylmethyl}-thioacetamide

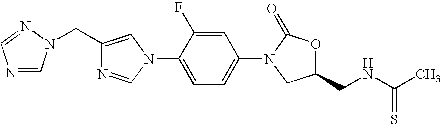

The title compound is prepared by following the procedure as described in example 60, by taking appropriate starting materials.

Yield: 40%.

Melting Range: 171-173° C.,

MS (m/z): 416 (M⁺+1), 347,

¹H NMR (300 MHz, CDCl₃): δ 8.28 (s, 1H), 7.89 (s, 1H), 7.63-7.73 (m, 2H), 7.22-7.38 (m, 3H), 5.31 (s, 2H), 4.91-5.00 (m, 1H), 3.96-4.16 (m, 3H), 3.87 (t, J=5.3 Hz, 1H), 2.51 (s, 2H). {3-[3-Fluoro-4-(4-pyrazol-1-ylmethyl-[1,2,3]triazol-1-yl)-phenyl]-5-hydroxy methyl-oxazolidin-2-one could be converted into N-{3-[3-fluoro-4-(4-pyrazol-1-yl-methyl-(1,2,3)triazol-1-yl)-phenyl]-2-oxo-oxazolidin-5-ylmethyl}acetamide or {3-[3-fluoro-4-(4-pyrazol-1-yl-methyl-[1,2,3]triazol-1-yl)-phenyl]-2-oxo-oxazolidin-5-yl-methyl}-thiocarbamic acid methyl ester or N-{3-[3-fluoro-4-(4-pyrazol-1-yl-methyl-(1,2,3)triazol-1-yl)-phenyl]-2-oxo-oxazolidin-5-ylmethyl}carbamic acid methyl ester according to the procedures depicted above.

### Example 68

### (S)-2,2-Difluoro-N-{3-[3-fluoro-4-(4-[1,2,4]triazol-1-ylmethyl-imidazol-1-yl)-phenyl]-2-oxo-oxazolidin-5-ylmethyl}-acetamide

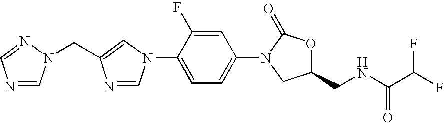

To a solution of 5-Aminomethyl-3-[3-fluoro-4-(4-[1,2,4]triazol-1-ylmethyl-imidazol-1-yl)-phenyl]-oxazolidin-2-one (355 mg, 0.99 mmol) in DCM was added N-methyl morpholin (151 mg, 1.49 mmol) and difluoroacetic acid (0.06 mL, 0.99 mmol) at 0° C. dropwise followed by the addition of EDC.HCl (247 mg, 1.29 mmol). The resulting mixture was stirred for 3 hours at room temperature. After the completion of the reaction, reaction mixture was extracted with DCM, washed with water and brine. The organic layer was dried over Na₂SO₄ and concentrated. The crude product was purified by column chromatography to obtain the desired product as white solid (100 mg)

Yield: 23%

IR (KBr, cm⁻¹): 3403, 1739, 1708, 1531, 1426, 1271, 1129, 1017

¹HNMR (400 MHz, DMSO): δ 9.16 (t, J=5.6 Hz, 1H), 8.56 (s, 1H), 7.99 (s, 1H), 7.94 (s, 1H), 7.72 (dd, J=2.4, 13.4 Hz, 1H), 7.67 (t, J=8.9 Hz, 1H), 7.56 (s, 1H), 7.46-7.45 (m, 1H), 6.25 (t, J=53.4 Hz, 1H), 5.34 (s, 2H), 4.87-4.82 (m, 1H), 4.21 (t, J=9.1 Hz, 1H), 3.83 (dd, J=6.2, 9.1 Hz, 1H), 3.56 (t, J=5.6 Hz, 2H)

ES-MS (m/z): 436 (M⁺+1)

### Example 69

### (S)-2,2-Dichloro-N-{3-[3-fluoro-4-(4-[1,2,4]-triazol-1-ylmethyl-imidazol-1-yl)-phenyl]-2-oxo-oxazolidin-5-ylmethyl}-acetamide

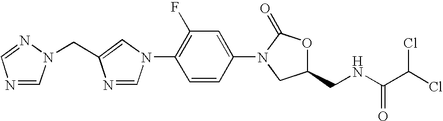

The title compound was obtained from 5-Aminomethyl-3-[3-fluoro-4-(4-[1,2,4]triazol-1-ylmethyl-imidazol-1-yl)-phenyl]-oxazolidin-2-one and dichloroacetic acid following the procedure described for (S)-2,2-Difluoro-N-{3-[3-fluoro-4-(4-[1,2,4]triazol-1-ylmethyl-imidazol-1-yl)-phenyl]-2-oxo-oxazolidin-5-ylmethyl acetamide.

Yield: 25%

¹H NMR (400 MHz, DMSO): δ 8.97 (t, J=5.6 Hz, 1H), 8.56 (s, 1H), 7.98 (s, 1H), 7.94 (s, 1H), 7.72 (dd, J=2.4, 13.4 Hz, 1H), 7.67 (t, J=8.9 Hz, 1H), 7.56 (s, 1H), 7.45-7.44 (m, 1H), 6.48 (s, 1H), 5.34 (s, 2H), 4.87-4.84 (m, 1H), 4.21 (t, J=9.1 Hz, 1H), 3.83-3.78 (m, 1H), 3.56 (t, J=5.4 Hz, 2H)

ES-MS (m/z): 468 (M⁺)

### Example 70

### (S)-2,2-Dichloro-N-{3-[3-fluoro-4-(4-pyrazol-1-ylmethyl-imidazol-1-yl)-phenyl]-2-oxo-oxazolidin-5-ylmethyl}-propionamide

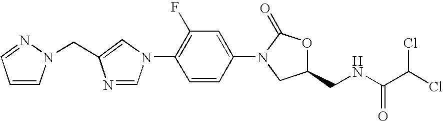

The title compound was obtained from the corresponding amine using the same procedure described for the preparation of (S)-2,2-Dichloro-N-{3-[3-fluoro-4-(4-[1,2,4]-triazol-1-ylmethyl-imidazol-1-yl)-phenyl]-2-oxo-oxazolidin-5-ylmethyl}acetamide.

IR (KBr, cm⁻¹): 3248, 3114, 3063, 1757, 1709, 1530, 1419, 1214, 1087, 814, 752

¹HNMR (400 MHz, DMSO): δ 8.96 (t, J=5.6 Hz, 1H), 7.97 (s, 1H), 7.80-7.70 (m, 2H), 7.66 (t, J=8.8 Hz, 1H), 7.47 (s, 1H), 7.43 (dd, J=1.6, 9.1 Hz, 2H), 6.49 (s, 1H), 6.24 (t, J=1.9 Hz, 1H), 5.25 (s, 1H), 4.90-4.80 (m, 1H), 4.20 (t, J=9.1 Hz, 1H), 3.80 (dd, J=6.2, 9.4 Hz, 1H), 3.39-3.37 (m, 2H)

ES-MS (m/z): 467 (M⁺+1)

### Example 71

### N-(3-{3-Fluoro-4-[4-(4-pyridin-3-yl-imidazol-1-ylmethyl)-[1,2,3]triazol-1-yl]-phenyl}-2-oxo-oxazolidin-5-ylmethyl)-acetamide

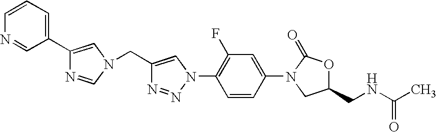

The title compound is prepared by following the procedure as described in example 28, by taking appropriate starting materials.

Yield: 51%

IR (KBr, cm⁻¹): 3282, 1754, 1737, 1670, 1531, 1408, 1236, 1050, 751

¹H NMR (400 MHz, DMSO), δ 8.96 (d, J=2.1 Hz, 1H), 8.65 (d, J=1.9 Hz, 1H), 8.39 (d, J=4.8 Hz, 1H), 8.24 (t, J=5.6 Hz, 1H), 8.09 (dt, J₁=7.8 Hz, J₂=1.9 Hz, 1H), 7.91 (d, J=1.1 Hz, 1H), 7.89-7.78 (m, 3H), 7.55-7.52 (m, 1H), 7.38-7.34 (m, 1H), 5.45 (s, 2H), 4.80-4.77 (m, 1H), 4.19 (t, J=9.1 Hz, 1H), 3.81 (dd, J₁=6.4 Hz, J₂=8.4 Hz, 1H), 3.44 (t, J=5.6 Hz, 2H), 1.90 (s, 3H)

ES-MS (m/z): 477 (M⁺+1)

### Example 72

### (S)-N-(3-{4-[4-(3-Cyano-pyrrol-1-ylmethyl)-[1,2,3]triazol-1-yl]-3-fluoro-phenyl}-2-oxo-oxazolidin-5-ylmethyl)-acetamide

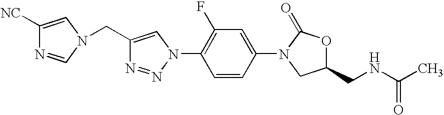

The title compound is prepared by following the procedure as described in example 28, by taking appropriate starting materials.

Yield: 50%

IR (KBr, cm⁻¹): 3332, 2229, 1759, 1667, 1528, 1410, 1205, 721

1H NMR (400 MHz, DMSO): 8.59 (d, J=1.9 Hz, 1H), 8.23 (t, J=5.9 Hz, 1H), 7.88-7.83 (m, 2H), 7.72 (t, J=1.8 Hz, 1H), 7.55-7.52 (m, 1H), 7.05 (d, J=2.9 Hz, 1H), 6.48 (d, J=2.9 Hz, 1H), 5.37 (s, 2H), 4.82-4.78 (m, 1H), 4.19 (t, J=9.1 Hz, 1H), 3.82-3.79 (m, 1H), 3.44 (t, J=5.4 Hz, 2H), 1.83 (s, 3H)

ES-MS (m/z): 424 (M⁺+1)

### Example 73

### (S)-N-(3-{3-Fluoro-4-[4-(4-methyl-thiazol-2-ylmethyl)-imidazol-1-yl]-phenyl}-2-oxo-oxazolidin-5-ylmethyl)-acetamide

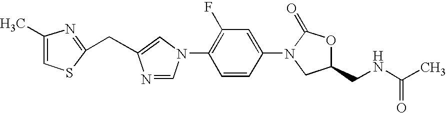

To a solution of N-{3-[3-Fluoro-4-(4-thiocarbamoylmethyl-imidazol-1-yl)-phenyl]-2-oxo-oxazolidin-5-ylmethyl}-acetamide (200 mg, 0.51 mmol) in ethanol was added chloro acetone (0.02 mL, 0.77 mmol) at room temperature. The reaction mixture was refluxed for overnight. The solvent was evaporated under reduced pressure and the residue was purified by column to afford the title product (30 mg)

Yield: 14%

IR (Neat, cm⁻¹): 1753, 1528, 1414, 1255, 1217, 1063, 759

1H NMR (400 MHz, DMSO): δ 8.23 (t, J=5.9 Hz, 1H), 7.96 (s, 1H), 7.75 (dd, J=2.7, 13.7 Hz, 1H), 7.66 (t, J=8.9 Hz, 1H), 7.46-4.44 (m, 2H), 7.08 (d, J=1.0 Hz, 1H), 4.78-4.75 (m, 1H), 4.21 (s, 2H), 4.17 (t, J=9.1 Hz, 1H), 3.78 (dd, J=6.4, 9.1 Hz, 1H), 3.43 (t, J=5.4 Hz, 2H), 3.29 (s, 3H), 1.84 (s, 3H).

ES-MS (m/z): 430 (M⁺+1)

### Example 74

### (S)-N-{3-[3-Fluoro-4-(5-[1,2,4]-triazol-1-ylmethyl-[1,3,4]thiadiazol-2-yl)-phenyl]-2-oxo-oxazolidin-5-ylmethyl}-acetamide

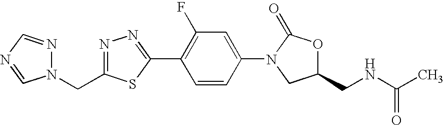

To a solution of 1H-[1,2,4]-triazole (32 mg, 0.47 mmol) and potassium carbonate (320 mg, 2.32 mmol) in dry DMF at 0° C. N-{3-[4-(5-Chloromethyl-[1,3,4]thiadiazol-2-yl)-3-fluoro-phenyl]-2-oxo-oxazolidin-5-ylmethyl}-acetamide (60 mg, 0.16 mmol) in DMF was added dropwise slowly and stirred at room temperature for overnight. After the completion of the reaction, reaction mixture was extracted with ethyl acetate for 3 times. The usual work up and purification afforded the desired compound as a white solid (45 mg).

Yield: 69%

IR (KBr, cm⁻¹): 3327, 3117, 1734, 1655, 1533, 1423, 1229, 862, 752, 673

¹H NMR (400 MHz, DMSO): δ 8.78 (s, 1H), 8.27 (t, J=8.6 Hz, 1H), 8.231 (t, J=6.2 Hz, 1H), 8.09 (s, 1H), 7.74 (dd, J=2.1, 13.7 Hz, 1H), 7.54 (dd, J=2.4, 8.9 Hz, 1H), 6.06 (s, 2H), 4.80-4.75 (m, 1H), 4.19 (t, J=9.1 Hz, 1H), 3.81 (dd, J=6.4 Hz, 9.1 Hz, 1H), 3.45 (t, J=5.6 Hz, 2H), 1.83 (s, 3H).

ES-MS (m/z): 418 (M⁺+1)

### Example 75

### (S)-N-{3-[(3-Fluoro-4-(5-imidazol-1-ylmethyl-[1,3,4]thiadiazol-2-yl)-phenyl]-2-oxo-oxazolidin-5-ylmethyl}-acetamide

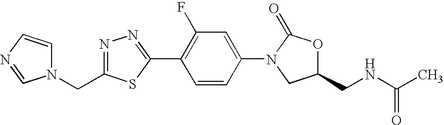

The title compound was prepared employing the procedure described for (S)-N-{3-[3-Fluoro-4-(5-[1,2,4]triazol-1-ylmethyl-[1,3,4]thiadiazol-2-yl)-phenyl]-2-oxo oxazolidin-5-ylmethyl}-acetamide.

Yield: 74%

¹H NMR (400 MHz, DMSO): δ 8.26 (t, J=8.6 Hz, 1H), 8.22 (t, J=5.9 Hz, 1H), 7.86 (s, 1H), 7.71 (dd, J=2.1, 14.0 Hz, 1H), 7.56 (dd, J=2.1, 8.9 Hz, 1H), 7.33 (s, 1H), 6.97 (s, 1H), 5.82 (s, 2H), 4.80-4.75 (m, 1H), 4.19 (t, J=9.1 Hz, 1H), 3.81 (dd, J=6.4 Hz, 9.1 Hz, 1H), 3.44 (t, J=5.3 Hz, 2H), 1.83 (s, 3H).

ES-MS (m/z): 417 (M⁺+1)

### Example 76

### (S)-tert-butyl (3-(3-fluoro-4-(5-(pyridin-2-ylmethyl)-1,3,4-thiadiazol-2-yl)phenyl)-2-oxooxazolidin-5-yl)methylcarbamate

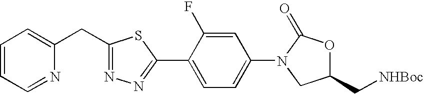

To a solution of (S)-tert-butyl (3-(3-fluoro-4-(2-(2-(pyridin-2-yl)acetyl)hydrazinecarbonyl)phenyl)-2-oxooxazolidin-5-yl)methylcarbamate (280 mg, 0.57 mmol) in dioxane, Lawesson's reagent (279 mg, 0.69 mmol) was added and refluxed for 3 hours. The solvent was evaporated under reduced pressure and the residue was purified by column chromatography to obtain the titled product 100 mg.

Yield: 36%.

IR (KBr, cm⁻¹): 3325, 2928, 1759, 1707, 1595, 1506, 1410, 1290, 1252, 1151, 1028, 914, 806, 753, 682

¹HNMR (400 MHz, DMSO): δ 8.55 (d, J=4.8 Hz, 1H), 8.24 (t, J=8.9 Hz, 1H), 7.82 (dd, J=1.9 Hz, 7.8 Hz, 1H), 7.74 (dd, J=6.7 Hz, 9.1 Hz, 1H), 7.58-7.50 (m, 2H), 7.38-7.30 (m, 1H), 7.20 (bs, 1H), 4.82-4.72 (m, 1H), 4.70 (s, 2H), 4.18 (t, J=8.9 Hz, 1H), 3.92-3.86 (m, 1H), 3.80-3.72 (m, 3H), 1.34 (s, 9H).

ES-MS (m/z): 486 (M⁺+1)

### Example 77

### (S)-5-(aminomethyl)-3-(3-fluoro-4-(5-(pyridin-2-ylmethyl)-1,3,4-thiadiazol-2-yl)phenyl)oxazolidin-2-one

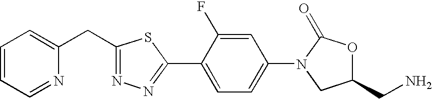

(S)-tert-butyl (3-(3-fluoro-4-(5-(pyridin-2-ylmethyl)-1,3,4-thiadiazol-2-yl)phenyl)-2-oxooxazolidin-5-yl)methylcarbamate (300 mg) was treated with 60% TFA in dichloromethane for 1 hours. The solvent was evaporated with toluene under reduced pressure and the residue was taken for next step (170 mg).

### Example 78

### (S)-N-((3-(3-fluoro-4-(5-(pyridin-2-ylmethyl)-1,3,4-thiadiazol-2-yl)phenyl)-2-oxooxazolidin-5-yl)methyl)acetamide

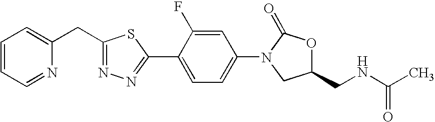

(S)-5-(aminomethyl)-3-(3-fluoro-4-(5-(pyridin-2-ylmethyl)-1,3,4-thiadiazol-2-yl)phenyl)oxazolidin-2-one (170 mg, 0.44 mmol) was acylated following the standard procedure to yield 70 mg of the required product.

IR (KBr, cm⁻¹): 3368, 1755, 1682, 1624, 1520, 1420, 1288, 1219, 862, 822, 752

¹HNMR (400 MHz, DMSO): δ 8.59-8.57 (m, 1H), 8.27-8.21 (m, 2H), 7.83 (dd, J=1.9 Hz, 7.5 Hz, 1H), 7.72 (dd. J=2.1 Hz, 14.0 Hz, 1H), 7.53 (dd, J=2.4 Hz, 8.8 Hz, 2H), 7.34-7.31 (m, 1H), 4.80-4.76 (m, 1H), 4.70 (s, 2H), 4.19 (t, J=9.1 Hz, 1H), 3.81 (dd, J=6.7 Hz, 9.4 Hz, 1H), 3.44 (t, J=5.4 Hz, 2H), 1.83 (s, 3H)

ES-MS (m/z): 428 (M⁺+1)

### Example 79

### (S)-N-{3-[3-Fluoro-4-(4-imidazol-1-ylmethyl-[1,2,3]triazol-1-yl)-phenyl]-2-oxo-oxazolidin-5-ylmethyl}-acetamide

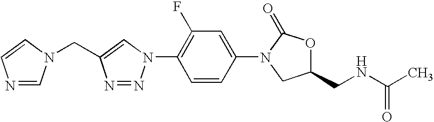

The title compound is prepared by following the procedure as described in example 28, by taking appropriate starting materials.

Yield: 55%

IR (KBr, cm⁻¹): 3252, 1754, 1673, 1532, 1417, 1218, 1048, 756

¹HNMR (400 MHz, DMSO): δ 8.59 (s, 1H), 8.24 (bs, 1H), 7.82-7.78 (m, 3H), 7.54 (dd, J=1.6, 8.9 Hz, 1H), 7.26 (s, 1H), 6.93 (s, 1H), 5.39 (s, 2H), 4.82-4.75 (m, 1H), 4.19 (t, J=8.9 Hz, 1H), 3.82 (dd, J=6.4, 9.1 Hz, 1H), 3.44 (t, J=5.6 Hz, 2H), 1.84 (s, 3H).

ES-MS (m/z): 400 (M⁺+1)

### Example 80

### (S)-N-((3-(3-fluoro-4-(4-(hydroxy(pyridin-2-yl)methyl)-1H-1,2,3-triazol-1-yl)phenyl)-2-oxooxazolidin-5-yl)methyl)acetamide

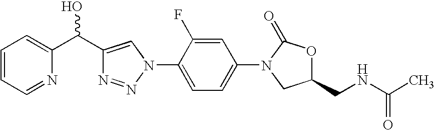

The title compound is prepared by following the procedure as described in example 28, by taking appropriate starting materials.

Yield: 46%

IR (Neat, cm⁻¹): 3308, 3291, 1755, 1661, 1526, 1408, 1227, 1211, 1039, 752

¹HNMR (400 MHz, DMSO): δ 8.48 (d, J=4.3 Hz, 1H), 8.36 (d, J=1.4 Hz, 1H), 8.23 (t, J=5.9 Hz, 1H), 7.86-7.80 (m, 2H), 7.76 (d, J=2.1 Hz, 1H), 7.61 (d, J=8.0 Hz, 1H), 7.51 (d, J=8.8 Hz, 1H), 7.34-7.18 (m, 1H), 6.24 (d, J=5.1 Hz, 1H), 5.93 (d, J=5.1 Hz, 1H), 4.84-4.75 (m, 1H), 4.18 (t, J=8.8 Hz, 1H), 3.81 (t, J=5.4 Hz, 1H), 3.44 (t, J=5.3 Hz, 2H), 1.83 (s, 3H)

ES-MS (m/z): 427 (M⁺+1)

### Example 81

### (S)-N-((3-(3-fluoro-4-(4-(hydroxy(pyridin-3-yl)methyl)-1H-1,2,3-triazol-1-yl)phenyl)-2-oxooxazolidin-5-yl)methyl)acetamide

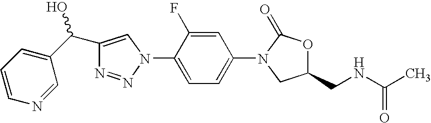

The title compound is prepared by following the procedure as described in example 28, by taking appropriate starting materials.

Yield: 65%

IR (KBr, cm⁻¹): 3273, 1755, 1658, 1525, 1481, 1406, 1220, 1213, 1145, 1124, 752, 711, 663, 628, 567

¹HNMR (400 MHz, DMSO): δ 8.68 (bs, 1H), 8.50 (bs, 1H), 8.43 (d, J=1.8 Hz, 1H), 8.24 (t, J=5.9 Hz, 1H), 7.88-7.76 (m, 3H), 7.52 (dd, J=1.8 hz, 8.8 Hz, 1H), 7.39 (dd, J=4.5 Hz, 8.0 Hz, 1H), 6.30 (d, J=4.8 Hz, 1H) 6.01 (d, J=4.8 Hz, 1H), 4.82-4.74 (m, 1H), 4.19 (t, J=8.8 Hz, 1H), 3.81 (dd, J=6.4, 9.1 Hz, 1H), 3.44 (t, J=5.6 Hz, 2H), 1.84 (s, 3H).

ES-MS (m/z): 427 (M⁺+1)

### Example 82

### (S)-N-((3-(3-fluoro-4-(4-(pyridin-2-ylmethyl)-1H-1,2,3-triazol-1-yl)phenyl)-2-oxooxazolidin-5-yl)methyl)acetamide

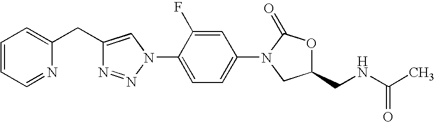

A mixture of (S)-N-((3-(3-fluoro-4-(4-(hydroxy(pyridin-2-yl)methyl)-1H-1,2,3-triazol-1-yl)phenyl)-2-oxooxazolidin-5-yl)methyl)acetamide (200 mg, 0.42 mmol), glacial acetic acid (10 mL), 10% Pd—C and conc. HCl (1 mL) was stirred at room temperature for 40 hours under hydrogen atmosphere. The reaction mixture was filtered over celite, the filterate was basified with aq. NH₃ solution and extracted with ethyl acetate. The organic layer was dried over anhyd. Na₂SO₄ and concentrated under reduced pressure. The residue was purified by column chromatography to obtain the titled product.

Yield: 24%

IR (KBr, cm⁻¹): 3265, 3165, 3094, 2932, 1749, 1593, 1433, 1406, 1047, 1005, 906, 816, 750, 679, 602.

¹HNMR (400 MHz, DMSO): δ 8.54-8.50 (m, 1H), 8.37 (d, J=1.9 Hz, 1H), 8.23 (t, J=5.6 Hz, 1H), 7.84 (t, J=8.6 Hz, 1H), 7.80-7.77 (m, 1H), 7.75 (dd, J=6.2 Hz, 8.0 Hz, 1H), 7.52 (dd, J=1.8 Hz, 1.9 Hz, 1H), 7.36 (d, J=7.7 Hz, 1H), 7.26-7.23 (m, 1H), 4.84-4.75 (m, 1H), 4.25 (s, 2H), 4.19 (t, J=9.1 Hz, 1H), 3.84-3.79 (m, 1H), 3.44 (t, J=5.6 Hz, 2H), 1.83 (s, 3H).

ES-MS (m/z): 411 (M⁺+1)

### Example 83

### (S)-N-((3-(3-fluoro-4-(4-(pyridin-3-ylmethyl)-1H-1,2,3-triazol-1-yl)phenyl)-2-oxooxazolidin-5-yl)methyl)acetamide

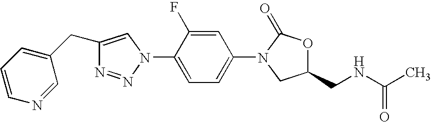

The title compound is prepared by following the procedure as mentioned for (S)-N-((3-(3-fluoro-4-(4-(pyridin-2-ylmethyl)-1H-1,2,3-triazol-1-yl)phenyl)-2-oxooxazolidin-5-yl)methyl)acetamide

Yield: 32%

IR (KBr, 3248, 3174, 3041, 2829, 1757, 1681, 1591, 1529, 1332, 1215, 1049, 821, 632

¹HNMR (400 MHz, DMSO): δ 8.56 (d, J=2.1 Hz, 1H), 8.44 (dd, J=1.5 Hz, 4.8 Hz, 1H), 8.39 (d, J=1.8 Hz, 1H), 8.23 (t, J=5.7 Hz, 1H), 7.84 (t, J=8.8 Hz, 1H), 7.78 (dd, J=2.4 Hz, 13.4 Hz, 1H), 7.75-7.69 (m, 1H), 7.51 (dd, J=1.8 Hz, 8.5 Hz, 1H), 7.34 (dd, J=4.5 Hz, 7.6 Hz, 1H), 4.82-4.74 (m, 1H), 4.18 (t, J=8.8 Hz, 1H), 4.14 (s, 2H), 3.84-3.76 (m, 1H), 3.44 (t, J=5.4 Hz, 2H), 1.83 (s, 3H)

ES-MS (m/z): 411 (M⁺+1)

### Example 84

### (S)-N-((3-(3-fluoro-4-(4-(pyridin-4-ylmethyl)-1H-1,2,3-triazol-1-yl)phenyl)-2-oxooxazolidin-5-yl)methyl)acetamide

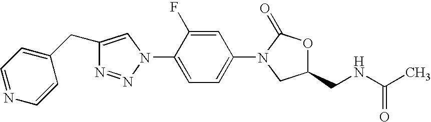

The title compound is prepared by following the procedure as mentioned for (S)-N-((3-(3-fluoro-4-(4-(pyridin-2-ylmethyl)-1H-1,2,3-triazol-1-yl)phenyl)-2-oxooxazolidin-5-yl)methyl)acetamide.

IR (KBr, cm⁻¹): 3277, 3154, 2320, 1748, 1651, 1599, 1531, 1414, 1215, 808, 588

¹HNMR (400 MHz, DMSO): δ 8.49 (dd, J=1.5 Hz, 4.6 Hz, 2H), 8.43 (d, J=1.8 Hz, 1H), 8.23 (t, J=5.8 Hz, 1H), 7.89-7.79 (m, 2H), 7.52 (dd, J=1.8 Hz, 8.9 Hz, 1H), 7.32 (d, J=5.8 Hz, 2H), 4.81-4.75 (m, 1H), 4.20 (t, J=9.2 Hz, 1H), 4.10 (s, 1H), 3.81 (dd, J=6.4 Hz, 9.2 Hz, 1H), 3.44 (t, J=5.2 Hz, 2H), 1.82 (s, 3H).

ES-MS (m/z): 411 (M⁺+1)

### Example 85

### (S)-N-{3-[3-Fluoro-4-(3-[1,2,4]triazol-1-ylmethyl-[1,2,4]oxadiazol-5-yl)-phenyl]-2-oxo-oxazolidin-5-ylmethyl}-acetamide

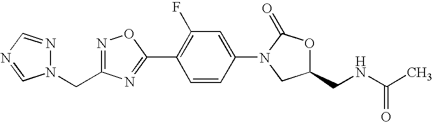

To a solution of 4-[5-(Acetylamino-methyl)-2-oxo-oxazolidin-3-yl]-2-fluoro-benzoic acid (200 mg, 0.67 mmol) in dry THF were added Hunig's base (87 mg, 0.67 mmol), EDC.HCl (130 mg, 0.67 mmol) and HOBT (91 mg, 0.67 mmol). The mixture was stirred for 5 minutes at room temperature and a solution of N-Hydroxy-2-[1,2,4]triazol-1-yl-acetamidine in THF was added at 0° C. The mixture was brought to room temperature and stirred for overnight. Reaction mixture was concentrated and extracted with ethylacetate and dried over Na₂SO₄ and concentrated. The crude compound was dissolved with dry DMF and heated to 110-120° C. for overnight. The usual work up and purification yielded the title compound (60 mg).

Yield: 22%

¹H NMR (400 MHz, DMSO): δ 8.74 (s, 1H), 8.22 (t, J=5.6 Hz, 1H), 8.11 (t, J=8.6 Hz, 1H), 8.04 (s, 1H), 7.73 (dd, J=2.1, 13.7 Hz, 1H), 7.58 (dd, J=2.1 Hz, 8.9 Hz, 1H), 5.75 (s, 2H), 4.81-4.77 (m, 1H), 4.19 (t, J=9.1 Hz, 1H), 3.82 (dd, J=6.2, 9.2 Hz, 1H), 3.44 (t, J=5.6 Hz, 2H), 1.83 (s, 3H).

ES-MS (m/z): 402 (M⁺+1)

### Example 86

### (S)-N-(3-{3-Fluoro-4-[1-(1-trityl-1H-imidazol-4-ylmethyl)-1H-[1,2,3]triazol-4-yl]-phenyl}-2-oxo-oxazolidin-5-ylmethyl)-acetamide

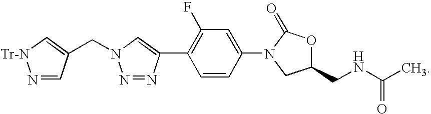

The title compound is prepared by following the procedure as mentioned for 4-Azidomethyl-1-trityl-1H-imidazole and N-[3-(4-Ethynyl-3-fluoro-phenyl)-2-oxo-oxazolidin-5-ylmethyl]-acetamide.

Yield: 47%

¹H NMR (400 MHz, DMSO): δ 8.26-8.19 (m, 2H), 8.11 (t, J=8.6 Hz, 1H), 7.64 (dd, J=2.1, 13.4 Hz, 1H), 7.44-7.35 (m, 11H), 7.16 (s, 1H), 7.11-7.07 (m, 6H), 5.51 (s, 2H), 4.76-4.73 (m, 1H), 4.16 (t, J=8.8 Hz, 1H), 3.82 (dd, J=6.4, 9.1 Hz, 1H), 3.43 (t, J=5.6 Hz, 2H), 1.84 (s, 3H).

ES-MS (m/z): 642 (M⁺+1)

### Example 87

### (S)-N-(3-{3-Fluoro-4-[1-(1H-imidazol-4-ylmethyl)-1H-[1,2,3]triazol-4-yl]-phenyl}-2-oxo-oxazolidin-5-ylmethyl)-acetamide

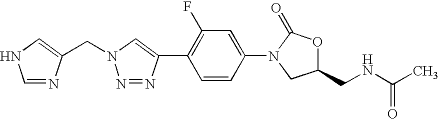

The compound N-(3-{3-Fluoro-4-[1-(1-trityl-1H-imidazol-4-ylmethyl)-1H-[1,2,3]triazol-4-yl]-phenyl}-2-oxo-oxazolidin-5-ylmethyl)-acetamide (200 mg, 0.31 mmol) was stirred with trifluoroacetic acid in dichloromethane for 1 hour. The azeotropic removal of TFA with toluene and the purification over silica gel afforded the title compound (90 mg).

Yield: 72%

IR (KBr, cm⁻¹): 3285, 1740, 1654, 1560, 1415, 1227, 1070, 751

¹H NMR (400 MHz, DMSO): δ 12.1 (bs, 1H), 8.27 (d, J=3.5 HZ, 1H), 8.22 (t, J=5.9 Hz, 1H), 8.10 (t, J=8.6 (s, 1H), 7.64 (d, J=0.8 Hz, 1H), 7.61 (d, J=2.1 Hz, 1H), 7.41 (dd, J=2.1, 8.6 Hz, 1H), 7.23 (s, 1H), 5.54 (s, 2H), 4.80-4.75 (m, 1H), 4.16 (t, J=9.1 Hz, 1H), 3.78 (dd, J=6.5, 9.1 Hz, 1H), 3.43 (t, J=5.4 Hz, 2H), 1.84 (s, 3H)

ES-MS (m/z): 400 (M⁺+1)

### Example 88

### (S)-N-(3-{4-[1-(1-Cyanomethyl-1H-imidazol-4-ylmethyl)-1H-[1,2,3]triazol-4-yl]-3-fluoro-phenyl}-2-oxo-oxazolidin-5-ylmethyl)-acetamide

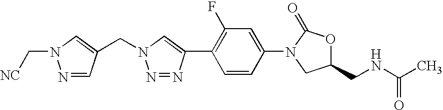

To a solution of N-(3-{3-Fluoro-4-[1-(1H-imidazol-4-ylmethyl)-1H-[1,2,3]triazol-4-yl]-phenyl}-2-oxo-oxazolidin-5-ylmethyl)-acetamide (50 mg, 0.13 mmol) and potassium carbonate (86 mg, 0.63 mmol) in dry DMF at 0° C. bromoacetonitrile (60 mg, 0.50 mmol) was added drop wise slowly and stirred at room temperature for overnight. The usual work up and purification produced the title compound (35 mg).

Yield: 64%

IR (KBr, cm⁻¹): 3417, 1636, 1384, 1070, 754

¹H NMR (400 MHz, DMSO): δ 8.40, 8.32 (2d, J=3.7 Hz, 1H), 8.22 (t, J=5.6 Hz, 1H), 8.13-8.07 (m, 1H), 7.86, 7.77 (2s, 1H rotamers in the ratio 1:1), 7.63 (d, J=13.7 Hz, 1H), 7.43-7.41 (m, 1H), 7.16 (s, 1H), 5.82 (s, 1H), 5.54 (s, 1H), 5.44 (s, 1H), 5.33 (s, 1H), 4.82-4.73 (m, 1H), 4.16 (t, J=9.1 Hz, 1H), 3.80-3.76 (m, 1H), 3.43 (t, J=5.4 Hz, 2H), 1.83 (s, 3H)

ES-MS (m/z): 439 (M⁺+1)

### Example 89

### (S)-N-(3-{3-Fluoro-4-[4-(3-methyl-[1,2,4]-triazol-4-ylmethyl)-[1,2,3]triazol-1-yl]-phenyl}-2-oxo-oxazolidin-5-ylmethyl)-acetamide

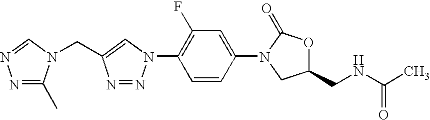

The title compound is prepared by following the procedure as described in example 28, by taking appropriate starting materials.

Yield: 57%

IR (KBr, cm⁻¹): 2936, 2676, 2493, 2360, 1752, 1528, 1475, 1398, 1036

1H NMR (400 MHz, DMSO): δ 8.62 (s, 1H), 8.48 (s, 1H), 8.31-8.21 (m, 1H), 7.85 (t, J=8.8 Hz, 1H), 7.80 (dd, J=2.1 Hz, 13.4 Hz, 1H), 7.53 (dd, J=2.5 Hz, 9.1 Hz, 1H), 5.39 (s, 2H), 4.85-4.75 (m, 1H), 4.19 (t, J=8.8 Hz, 1H), 3.89-3.78 (m, 1H), 3.44 (t, J=5.3 Hz, 2H), 1.83 (s, 3H)

ES-MS (m/z): 415 (M⁺+1)

### Example 90

### (S)-N-(3-{3-Fluoro-4-[4-(4-methyl-thiazol-2-ylmethyl)-[1,2,3]triazol-1-yl]-phenyl}-2-oxo-oxazolidin-5-ylmethyl)-acetamide

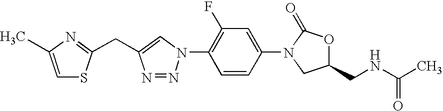

To a solution of N-{3-[4-(4-Cyanomethyl-[1,2,3]triazol-1-yl)-3-fluoro-phenyl]-2-oxo-oxazolidin-5-ylmethyl}-acetamide (200 mg, 0.56 mmol)) in pyridine, triethylamine (0.12 mL, 0.84 mmol) was added and passed H₂S gas at room temperature for two days. The reaction mixture was diluted with ethyl acetate and washed with water, brine and the organic layer was dried over Na₂SO₄. The solvent was evaporated under reduced pressure to obtain the crude thioamide (200 mg) which was dissolved in ethanol and treated with chloroacetone (59 mg, 0.76 mmol). The resulting mixture was refluxed for overnight. The solvent was evaporated under reduced pressure and the residue was purified by column chromatography to afford the title compound (80 mg).

Yield: 33%

IR (Neat, cm⁻¹): 3316, 2924, 1745, 1527, 1408, 1228, 702

1H NMR (400 MHz, DMSO): 8.49 (d, J=1.9 Hz, 1H), 8.24 (t, J=5.7 Hz, 1H), 7.86 (t, J=8.7 Hz, 1H), 7.78 (dd, J=2.1, 13.4 Hz, 1H), 7.52 (dd, J=1.6, 8.9 Hz, 1H), 7.14 (d, J=1.1 Hz, 1H), 4.82-4.75 (m, 1H), 4.46 (s, 2H), 4.19 (t, J=9.0 Hz, 1H), 3.81 (dd, J=6.4, 9.1 Hz, 1H), 3.45 (t, J=5.5 Hz, 2H), 2.33 (d, J=0.8 Hz, 3H), 1.84 (s, 3H).

ES-MS (m/z): 431 (M⁺+1)

### Example 91

### (S)-N-{3-[3-Fluoro-4-(4-[1,2,4]oxadiazol-3-ylmethyl-[1,2,3]triazol-1-yl)-phenyl]-2-oxo-oxazolidin-5-ylmethyl}-acetamide

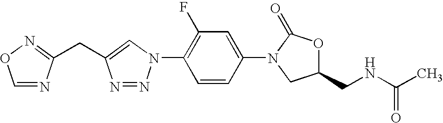

A solution of N-(3-{3-Fluoro-4-[4-(N-hydroxycarbamimidoylmethyl)-[1,2,3]triazol-1-yl]-phenyl}-2-oxo-oxazolidin-5-ylmethyl)-acetamide (1 gram, 2.56 mmol), BF₃.Et₂O (0.2 mL) in trimethyl orthoformate (20 mL) was refluxed at 120° C. The solvent was evaporated and the residue was purified by column chromatography to obtain the product as a white solid (600 mg).

Yield: 58%

IR (KBr, cm⁻¹): 3422, 2925, 1708, 1531, 1407, 1243, 1135, 1051, 1017, 678.

1H NMR (400 MHz, DMSO): δ9.56 (s, 1H), 8.48 (d, J=2.1 Hz, 1H), 8.23 (t, J=5.6 Hz, 1H), 7.84 (t, J=8.8 Hz, 1H), 7.80 (dd, J=2.4 Hz, 13.4 Hz, 1H), 7.53 (dd, J=1.6 Hz, 8.0 Hz, 1H), 4.84-4.7 (m, 1H), 4.34 (s, 2H), 4.19 (t, J=9.1 Hz, 1H), 3.87-3.78 (m, 1H), 3.44 (t, J=5.3 Hz, 2H), 1.84 (s, 3H).

ES-MS (m/z): 402 (M⁺+1)

### Example 92

### (S)-N-(3-{3-Fluoro-4-[4-(5-methyl-[1,2,4]oxadiazol-3-ylmethyl)-[1,2,3]-triazol-1-yl]-phenyl}-2-oxo-oxazolidin-5-ylmethyl)-acetamide

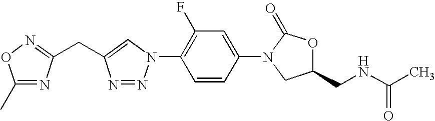

To a solution of N-(3-{3-Fluoro-4-[4-(N-hydroxycarbamimidoylmethyl)-[1,2,3]triazol-1-yl]-phenyl}-2-oxo-oxazolidin-5-ylmethyl)-acetamide (250 mg, 0.639 mmol) in pyridine acetic anhydride (0.071 mL, 0.767 mmol) was added and the reaction mixture was refluxed at 120° C. for overnight. Upon completion, the pyridine was removed under reduced pressure and the residue was purified by column chromatography to obtain the product as a light brown solid (190 mg).

Yield: 72%

IR (KBr, cm⁻¹): 3299, 1750, 1643, 1535, 1416, 1227, 1079, 1046, 874, 756, 602.

1H NMR (400 MHz, DMSO): δ 8.46 (s, 1H), 8.29-8.21 (m, 1H), 7.89-7.77 (m, 2H), 7.53 (dd, J=1.8 Hz, 9.1 Hz, 1H), 4.85-4.75 (m, 1H), 4.24 (s, 2H), 4.19 (t, J=9.1 Hz, 1H), 3.81 (t, J=6.7 Hz, 1H), 3.44 (t, J=5.6 Hz, 2H), 3.29 (s, 2H), 1.84 (s, 3H).

ES-MS (m/z): 416 (M⁺+1)

### Example 93

### (S)-N-{3-[3-Fluoro-4-(4-[1,2,3]-triazol-1-ylmethyl-[1,2,3]triazol-1-yl)-phenyl]-2-methylene-oxazolidin-5-ylmethyl}-acetamide

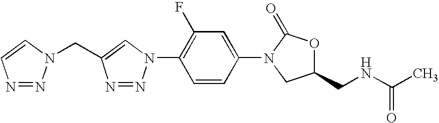

The title compound is prepared by following the procedure as described in example 28, by taking appropriate starting materials.

Yield: 42%

IR (KBr, cm⁻¹): 3301, 2924, 1748, 1646, 1528, 1458, 1212, 1053, 872, 745, 590.

1H NMR (400 MHz, DMSO): δ 8.66 (d, J=1.6 Hz, 1H), 8.30-8.19 (m, 2H), 7.85 (t, J=8.8 Hz, 1H), 7.80 (dd, J=2.4 Hz, 13.4 Hz, 1H), 7.76 (d, J=0.8 Hz, 1H), 7.58-7.52 (m, 1H), 5.82 (s, 2H), 4.84-4.75 (m, 1H), 4.19 (t, J=9.1 Hz, 1H), 3.87-3.78 (m, 1H), 3.44 (t, J=5.3 Hz, 2H), 1.84 (s, 3H).

ES-MS (m/z): 401 (M⁺+1)

### Example 94

### (S)-2,2-Dichloro-N-{3-[3-fluoro-4-(4-[1,2,4]-triazol-1-ylmethyl-[1,2,3]triazol-1-yl)-phenyl]-2-oxo-oxazolidin-5-ylmethyl}-acetamide

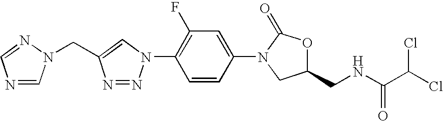

The title compound is prepared by following the procedure as described in example 68, by taking appropriate starting materials.

Yield: 43%

IR (KBr, cm⁻¹): 3422, 2925, 1708, 1531, 1407, 1243, 1135, 1051, 1017, 678.

1H NMR (400 MHz, DMSO): δ 8.97 (t, J=5.6 Hz, 1H), 8.66 (s, 1H), 8.60 (d, J=1.8 Hz, 1H), 7.98 (s, 1H), 7.85 (t, J=8.5 Hz, 1H), 7.79 (dd, J=2.4 Hz, 13.4 Hz, 1H), 7.52 (dd, J=1.6 Hz, 8.9 Hz, 1H), 6.48 (s, 1H), 5.61 (s, 2H), 4.93-4.84 (m, 1H), 4.23 (t, J=9.1 Hz, 1H), 3.82-3.80 (m, 1H), 3.57 (t, J=5.3 Hz, 2H).

ES-MS (m/z): 469 (M⁺+1)

### Example 95

### (S)-Cyclopropanecarboxylic acid {3-[3-fluoro-4-(4-[1,2,4]triazol-1-ylmethyl-[1,2,3]triazol-1-yl)-phenyl]-2-oxo-oxazolidin-5-ylmethyl}-amide

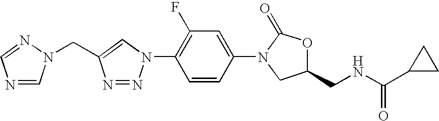

The title compound is prepared by following the procedure as described in example 68, by taking appropriate starting materials.

Yield: 51%

1H NMR (400 MHz, DMSO): δ 8.67 (s, 1H), 8.60 (d, J=1.9 Hz, 1H), 8.46 (t, J=5.6 Hz, 1H), 8.00 (s, 1H), 7.90-7.84 (m, 1H), 7.80 (dd, J=2.4 Hz, 13.4 Hz, 1H), 7.53 (dd, J=1.6 Hz, 9.1 Hz, 1H), 5.62 (s, 2H), 4.84-4.78 (m, 1H), 4.2 (t, J=9.1 Hz, 1H), 3.83 (dd, J=6.2 Hz, 9.4 Hz, 1H), 3.50-3.42 (m, 2H), 1.68-1.58 (m, 1H), 0.82-0.60 (m, 4H)

MS (m/z): 427 (M⁺+1)

### Example 96

### (S)-2-Cyano-N-{3-[3-fluoro-4-(4-[1,2,4]-triazol-1-ylmethyl-[1,2,3]triazol-1-yl)-phenyl]-2-oxo-oxazolidin-5-ylmethyl}-acetamide

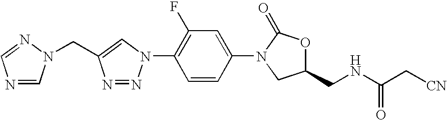

The title compound is prepared by following the procedure as described in example 68, by taking appropriate starting materials.

Yield: 10%

1H NMR (400 MHz, DMSO): δ 8.70 (s, 1H), 8.63 (t, J=5.6 Hz, 1H), 8.60 (d, J=1.9 Hz, 1H), 8.00 (s, 1H), 7.86 (t, J=8.9 Hz, 1H), 7.80 (dd, J=2.4 Hz, 13.4 Hz, 1H), 7.53 (dd, J=1.6 Hz, 9.1 Hz, 1H), 5.62 (s, 2H), 4.84-4.79 (m, 1H), 4.21 (t, J=9.1 Hz, 1H), 3.81 (dd, J=6.7 Hz, 9.4 Hz, 1H), 3.68 (s, 2H), 3.56-3.50 (m, 2H).

MS (m/z): 426 (M⁺+1)

### Example 97

### (R)-5-(azidomethyl)-3-(3-fluoro-4-(5-(pyridin-2-ylmethyl)-1,3,4-oxadiazol-2-yl)phenyl)oxazolidin-2-one

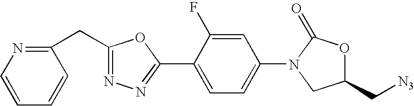

To a solution of (R)-4-(5-(azidomethyl)-2-oxooxazolidin-3-yl)-2-fluoro-N′-(2-(pyridin-2-yl)acetyl)benzohydrazide (300 mg, 0.72 mmol) in acetonitrile POCl₃ (57 mg, 0.36 mmol) and catalytic amount of DMAP were added and the reaction mixture was refluxed at 80° C. for overnight. Solvent was removed under reduced pressure and the residue was basified with saturated sodium carbonate and extracted with ethyl acetate. The organic layer was washed with brine and dried over sodium sulphate. Solvent was evaporated and the residue was purified by column chromatography to yield the title product 188 mg.

Yield: 65%

IR (KBr, cm⁻¹): 3070, 2924, 2094, 1629, 1492, 1409, 1209, 962, 748, 565

¹HNMR (400 MHz, DMSO): δ 8.52-8.48 (m, 1H), 8.00 (t, J=8.5 Hz, 1H), 7.82 (dt, J=1.8 Hz, 7.7 Hz, 1H), 7.72 (dd, J=2.1 Hz, 13.4 Hz, 1H), 7.57 (dd, J=2.1 Hz, 8.8 Hz, 1H), 7.48 (d, J=7.7 Hz, 1H), 7.34-7.30 (m, 1H), 4.98-4.90 (m, 1H), 4.54 (s, 2H), 4.20 (t, J=9.1 Hz, 1H), 3.85 (dd, J=6.1 Hz, 9.4 Hz, 1H), 3.78-3.70 (m, 2H)

ES-MS (m/z): 396 (M⁺+1)

### Example 98

### (S)-5-(aminomethyl)-3-(3-fluoro-4-(5-(pyridin-2-ylmethyl)-1,3,4-oxadiazol-2-yl)phenyl)oxazolidin-2-one

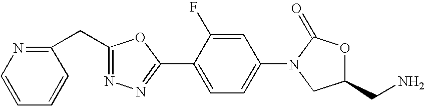

To a solution of (R)-5-(azidomethyl)-3-(3-fluoro-4-(5-(pyridin-2-ylmethyl)-1,3,4-oxadiazol-2-yl)phenyl)oxazolidin-2-one (200 mg) in methanol, 20% Pd—C was added and the reaction mixture was hydrogenated under balloon pressure for 3 hours. The solution was finally filtered over celite bed, the filterate was concentrated under reduced pressure. The crude residue was taken for the next step (150 mg).

### Example 99

### (S)-N-((3-(3-fluoro-4-(5-(pyridin-2-ylmethyl)-1,3,4-oxadiazol-2-yl)phenyl)-2-oxooxazolidin-5-yl)methyl)acetamide

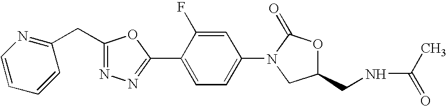

(S)-5-(aminomethyl)-3-(3-fluoro-4-(5-(pyridin-2-ylmethyl)-1,3,4-oxadiazol-2-yl)phenyl)oxazolidin-2-one (150 mg, 0.4 mmol) was acylated following the standard procedure to yield the required product 105 mg.

Yield: 63%.

IR (KBr, cm⁻¹): 3311, 3086, 2924, 1759, 1583, 1436, 1207, 1074, 995, 875, 817, 725, 673, 594

¹HNMR (400 MHz, DMSO): δ 8.52-8.49 (m, 1H), 8.22 (t, J=5.6 Hz, 1H), 7.99 (t, J=8.5 Hz, 1H), 7.82 (dt, J=1.8 Hz, 7.7 Hz, 1H), 7.70 (dd, J=2.1 Hz, 13.4 Hz, 1H), 7.53 (dd, J=2.1 Hz, 8.8 Hz, 1H), 7.48 (d, J=7.7 Hz, 1H), 7.34-7.30 (m, 1H), 4.82-4.74 (m, 1H), 4.54 (s, 2H), 4.19 (t, J=8.8 HZ, 1H), 3.80 (dd, J=6.7 Hz, 9.4 Hz, 1H), 3.43 (t, J=5.6 Hz, 2H), 1.83 (s, 3H).

ES-MS (m/z): 412 (M⁺+1)

### Example 100

### (S)-N-((3-(4-(5-((1H-1,2,4-triazol-1-yl)methyl)-1,3,4-oxadiazol-2-yl)-3-fluorophenyl)-2-oxooxazolidin-5-yl)methyl)acetamide

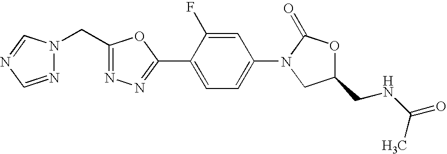

The title compound is prepared The title compound is prepared by following the procedure as described for (S)-N-((3-(3-fluoro-4-(5-(pyridin-2-ylmethyl)-1,3,4-oxadiazol-2-yl)phenyl)-2-oxooxazolidin-5-yl)methyl)acetamide.

¹HNMR (400 MHz, DMSO): δ 8.77 (s, 1H), 8.23 (t, J=5.6 Hz, 1H), 8.07 (s, 1H), 8.00 (t, J=8.6 Hz, 1H), 7.70 (dd, J=2.1 Hz, 13.7 Hz, 1H), 7.56 (dd, J=2.2 Hz, 8.9 Hz, 1H), 5.93 (s, 2H), 4.82-4.75 (m, 1H), 4.19 (t, J=8.8 Hz, 1H), 3.81 (dd, J=6.4 Hz, 9.1 Hz, 1H), 3.44 (t, J=5.4 Hz, 2H), 1.83 (s, 3H).

ES-MS (m/z): 402 (M⁺+1)

### Biology Data

The pharmaceutically acceptable compounds of the present invention are useful antibacterial agents having a good spectrum of activity against standard Gram-positive organisms.

### In Vitro:

Minimum Inhibiton Concentrations (MICs) were determined by broth microdilution technique as per the guidelines prescribed in the fifth edition of Approved Standards, NCCLS (currently CLSI) document M7-A5 Vol 20-No 2, 2000 Villinova, Pa. Initial stock solution of the test compound was prepared in DMSO. Subsequent two fold dilutions were carried out in sterile Mueller Hinton Broth (Difco) (MHB). Frozen cultures stocks were inoculated in 25 ml sterile MHB in 50 ml Erlyn Meyer flasks.

Composition of MHB is as follows: Beef Extract Powder—2.0 g/litre, Acid Digest of Casein—17.5 g/litre, Soluble Starch—1.5 g/litre, Final pH 7.3±0.1

Flasks were incubated for 4 to 5 hours at 36±1° C. on a rotary shaker at 110 rpm. Inoculum was prepared by diluting the culture in sterile MHB to obtain a turbidity of 0.5 McFarland standard. This corresponds to 1-2×10⁸ CFU/ml. The stock was further diluted in sterile broth to obtain 5×10⁴ to 1×10⁴ CFU/ml. 50 μl of the above diluted inoculum was added from 1-10 wells. The plates were incubated 18 to 20 hours at 37° C.

MIC is read as the lowest concentration of the compound that completely inhibits growth of the organism in the microdilution wells as detected by the unaided eye.

| Organism | Culture No. | DRCC No. |
|---|---|---|
| Staphylococcus aureus | ATCC 33591 | 019 |
| Staphylococcus aureus | ATCC 49951 | 213 |
| Staphylococcus aureus | ATCC 29213 | 035 |
| Enterococcus faecalis | ATCC 29212 | 034 |
| Enterococcus faecalis | NCTC 12201 | 153 |
| Enterococcus faecium | NCTC 12202 | 154 |
| Escherichia coli | ATCC 25922 | 018 |
| Haemophilus influenzae | ATCC 49247 | 432 |
| Haemophilus influenzae | ATCC 49766 | 433 |
| Haemophilus influenzae | ATCC 9006 | 529 |
| Moraxella catarrhalis | ATCC 25238 | 300 |
| Streptococcus pneumoniae | ATCC 6303 | 236 |
| Streptococcus pneumoniae | ATCC 49619 | 237 |
| Streptococcus pneumoniae | ATCC 700673 | 238 |
| Streptococcus mutans | | 561 |
| S. aureus - MRSA | — | 446 |
| S. aureus - MRSA | — | 448 |
| S. aureus - MRSA | — | 449 |
| Viridans Streptococci | | |

ATCC: American Type Culture Collection, USA
NCTC: National Collections of Type Cultures, Colindale, UK
DRCC: Dr. Reddy's Culture Collection, Hyderabad, India.
The in vitro antibacterial activity data is shown in TABLE 1.

TABLE 1

| | Minimum Inhibitory |
|---|---|
| Example Number | Concentration (MIC) (μg/ml) |
| 11 | 1-2 |
| 34 | 0.5-1 |
| 44 | 1-2 |
| 45 | 1-2 |
| 54 | 2-4 |
| 50 | 0.1-0.2 |
| 63 | 0.5 |
| 67 | 0.5-1 |

### In Vivo

Efficacies of the molecules were determined by systemic infection model. In this model, 6-7 weeks old Swiss albino mice (3 Males and 3 Females/group) weighing 21-25 grams were used. S. aureus ATCC 29213 and other strains were cultured on Columbia Blood agar (DIFCO), overnight, at 37° C. for 18 to 24 hours. Inoculum was prepared in 0.9% saline and optical density (OD) was adjusted at 560 nm to get 100×LD50 dose. Inoculum was diluted 1:1 with 10% Hog Gastric Mucin and 0.5 ml of inoculum was injected intraperitonially to each animal. Test compounds were solubilised in suitable formulation and administered by per-oral, subcutaneous or intra venous route by q.d., b.i.d. or t.i.d. protocol. Animals were observed for 5 to 7 days and survivals were noted. ED₅₀ Value (mg/kg) was calculated by Probit analysis.

The in vivo antibacterial activity data is shown in TABLE 2.

TABLE 2

| Example Number | ED₅₀ value (mg/kg) |
|---|---|
| 2 | 7.10 |
| 4 | 5.38 |
| 26 | 8.5 |
| 30 | 8.5 |
| 44 | 5.02 |
| 46 | 2.53 |

We claim:

1. A compound having the structure according to formula (I): 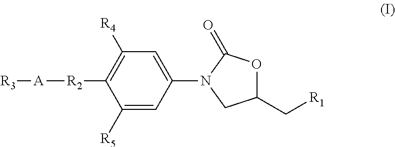
wherein:
R₁ is selected from hydroxy, amino, azido, C₁-C₈ alkyl, C₁-C₈ alkoxy, NHC(═Z)R, wherein Z is O or S, and R is hydrogen or optionally substituted alkyl, alkoxy, cycloalkyl or cycloalkoxy;
R₂ is a five-membered heterocyclic aromatic moiety containing one to three atoms selected from N, O and S;
A is —(CHR^{a})ₙ, wherein R^{a} represents hydrogen or hydroxyl, n represents 1-5;
R₃ is an optionally substituted five or six membered heteroaryl, having at least one nitrogen atom;
n represents 1-5;
R₄ and R₅ are independently selected from hydrogen or fluoro;
with the proviso that when R₁ is NHC(═O)CH₃, and R₂ is imidazole, R₃ is not a substituted triazole;
and including the stereoisomers, prodrugs, and pharmaceutically acceptable salts thereof.

2. The compound as claimed in claim 1,
wherein —R₃-A-R₂— represents

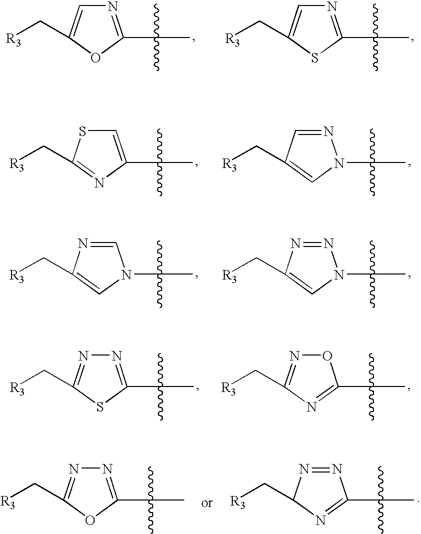

3. The compound as claimed in claim 2, wherein R₃ represents optionally substituted heteroaryl rings selected from

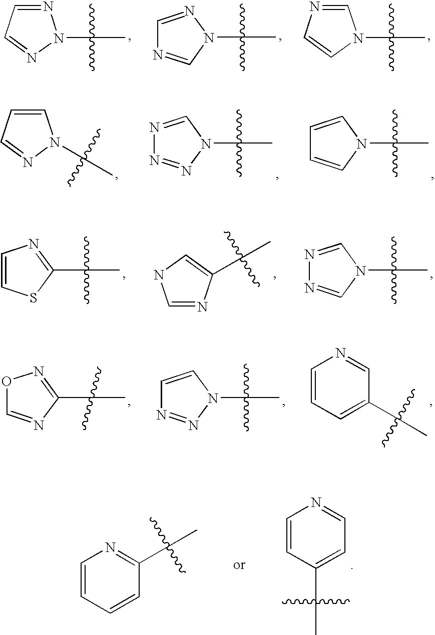

4. The compound as claimed in claim 3, wherein R₁ represents —NHC(═O)R, wherein R represents optionally substituted alkyl or cycloalkyl.

5. The compound as claimed in claim 3, wherein R₁ represents —NHC(═O)R, wherein R represents optionally substituted alkoxy or cycloalkoxy.

6. The compound as claimed in claim 3, wherein R₁ represents —NHC(═S)R, wherein R represents optionally substituted alkyl or cycloalkyl.

7. The compound as claimed in claim 3, wherein R₁ represents —NHC(═S)R, wherein R represents optionally substituted alkoxy or cycloalkoxy.

8. The compound as claimed in claim 1, is 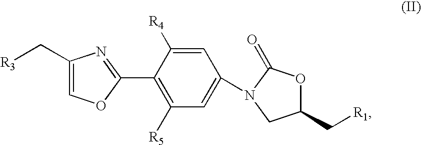
wherein R₁ and R₃ are as defined for formula (I)

9. The compound as claimed in claim 8, wherein R₃ represents optionally substituted

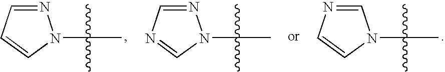

10. The compound as claimed in claim 1, is 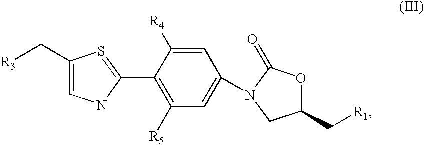
wherein R₁ and R₃ are as defined for formula (I)

11. The compound as claimed in claim 10, wherein R₃ represents optionally substituted

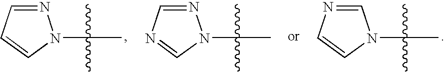

12. The compound as claimed in claim 1, is 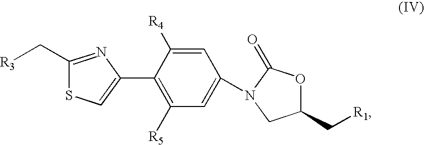
wherein R₁ and R₃ are as defined for formula (I).

13. The compound as claimed in claim 12, wherein R₃ represents optionally substituted

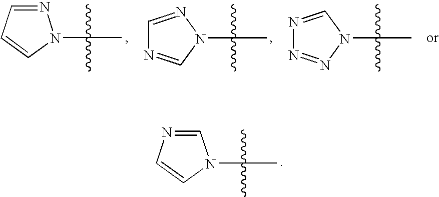

14. The compound as claimed in claim 1, is 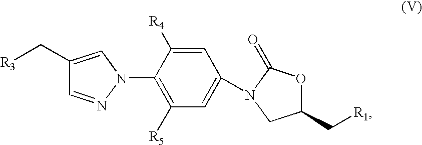
wherein R₁ and R₃ are as defined for formula (I).

15. The compound as claimed in claim 14, is

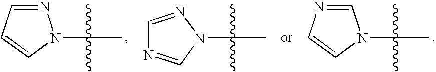

16. The compound as claimed in claim 1, is 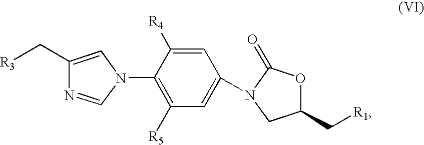
wherein R₁ and R₃ are as defined for formula (I).

17. The compound as claimed in claim 16, wherein R₃ represents optionally substituted

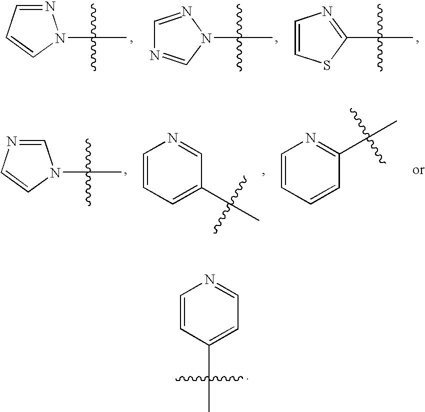

18. The compound as claimed in claim 1, is 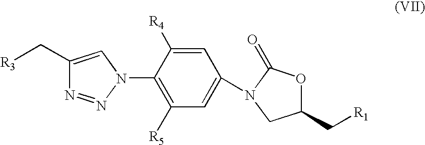
wherein R₁ and R₃ are as defined for formula (I).

19. The compound as claimed in claim 18, wherein R₃ represents optionally substituted

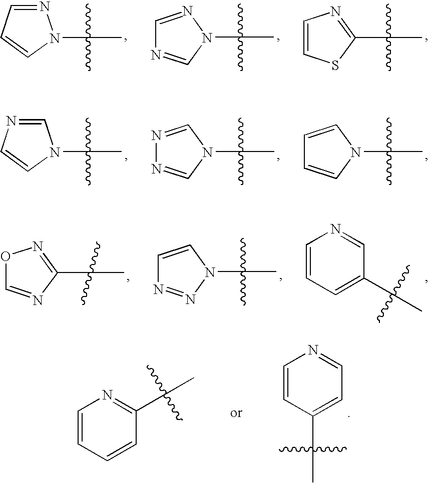

20. The compound as claimed in claim 1, is 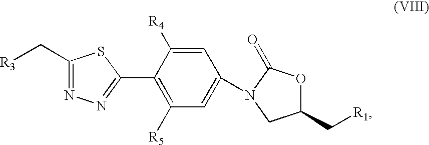
wherein R₁ and R₃ are as defined for formula (I).

21. The compound as claimed in claim 20, wherein R₃ represents optionally substituted

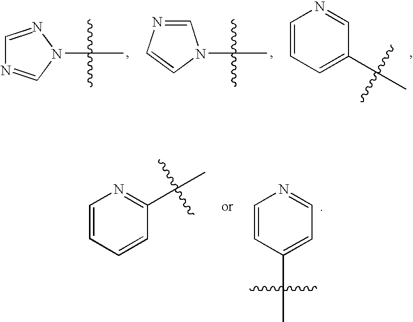

22. The compound as claimed in claim 1, is 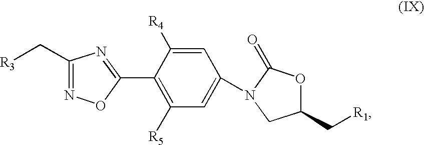
wherein R₁ and R₃ are as defined for formula (I).

23. The compound as claimed in claim 22, wherein R₃ represents optionally substituted

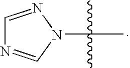

24. The compound as claimed in claim 1, is 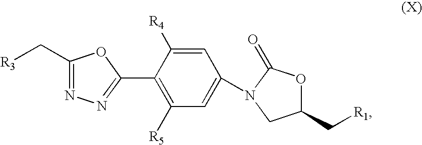
wherein R₁ and R₃ are as defined for formula (I).

25. The compound as claimed in claim 24, wherein R₃ represents optionally substituted.

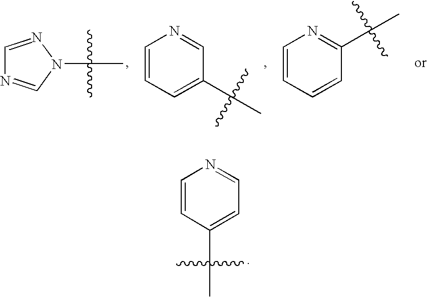

26. The compound as claimed in claim 1, is 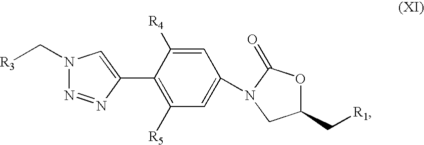
wherein R₁ and R₃ are as defined for formula (I).

27. The compound as claimed in claim 26, wherein R₃ represents optionally substituted

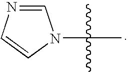

28. The compound as claimed claim 1, wherein the compound of formula (I) is selected from

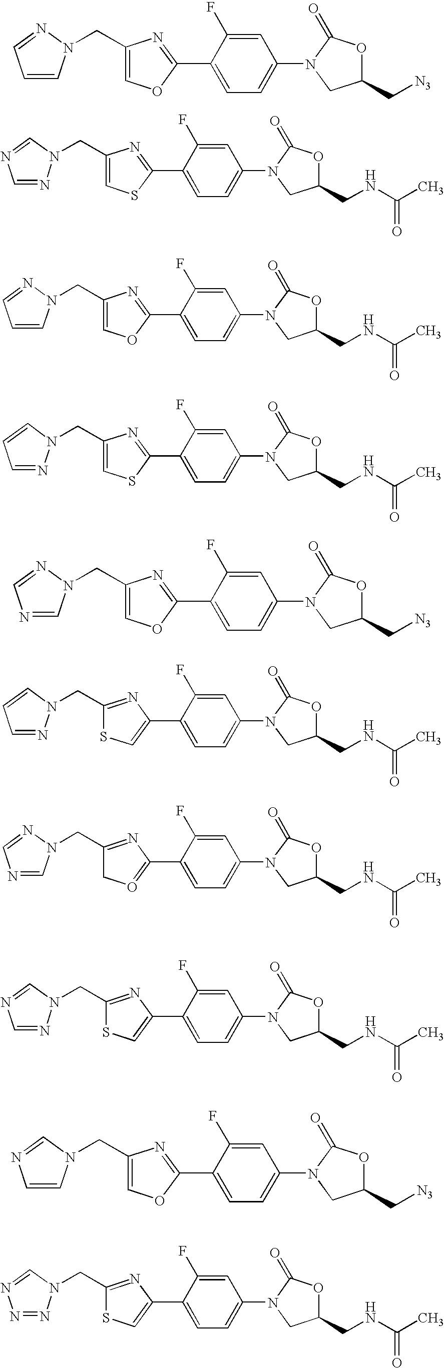

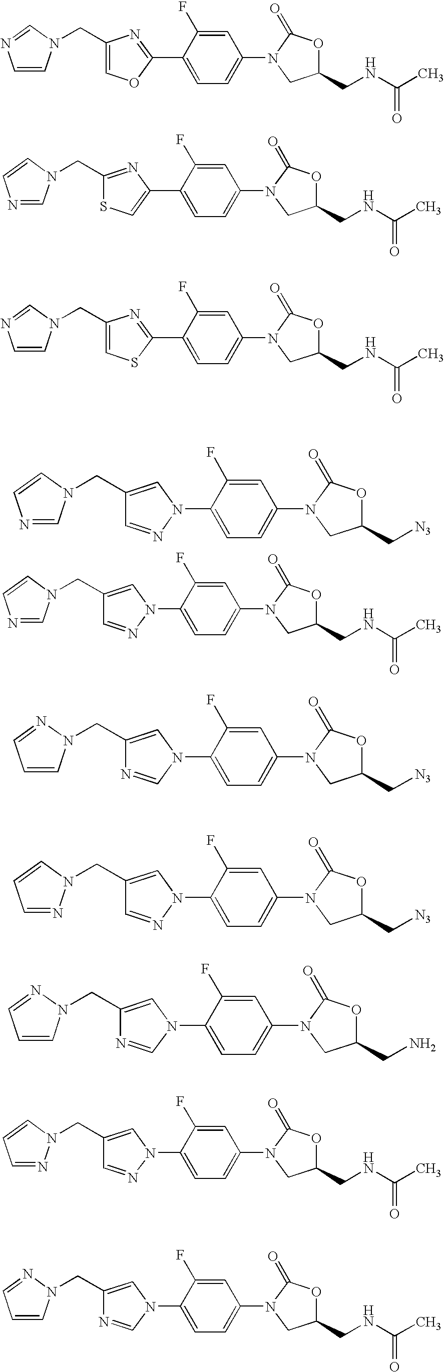

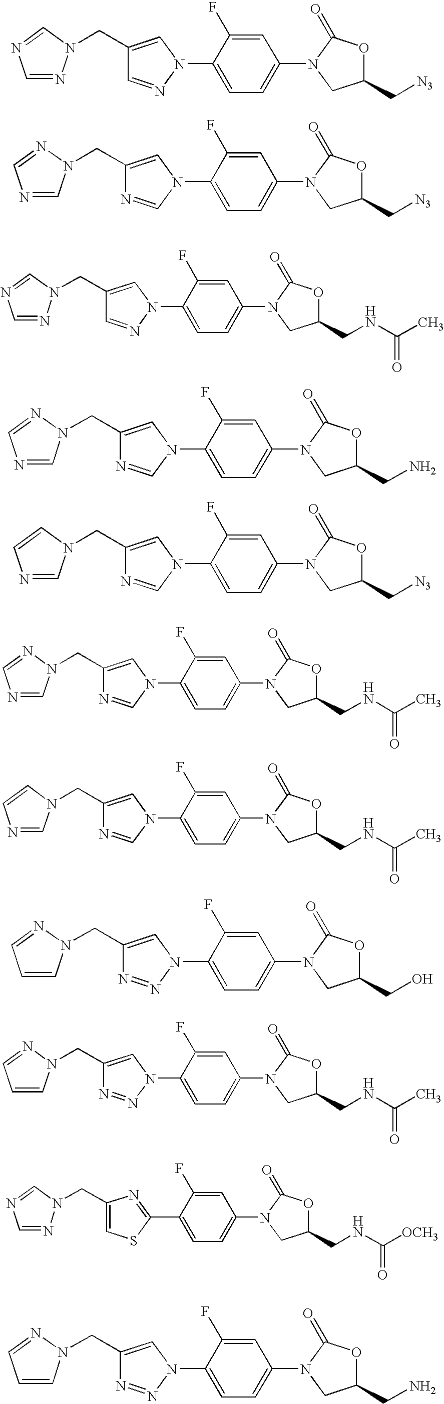

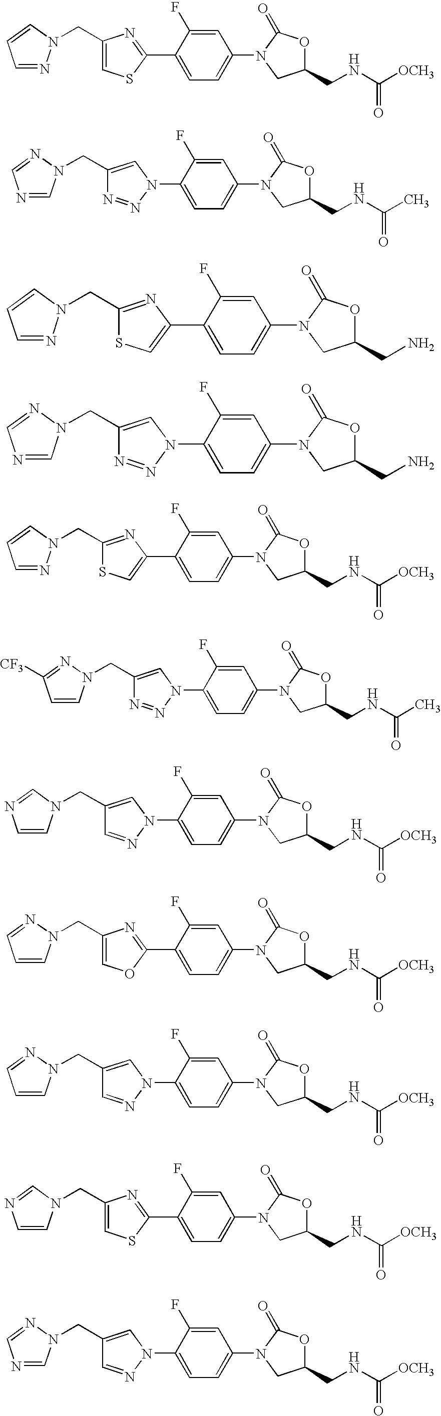

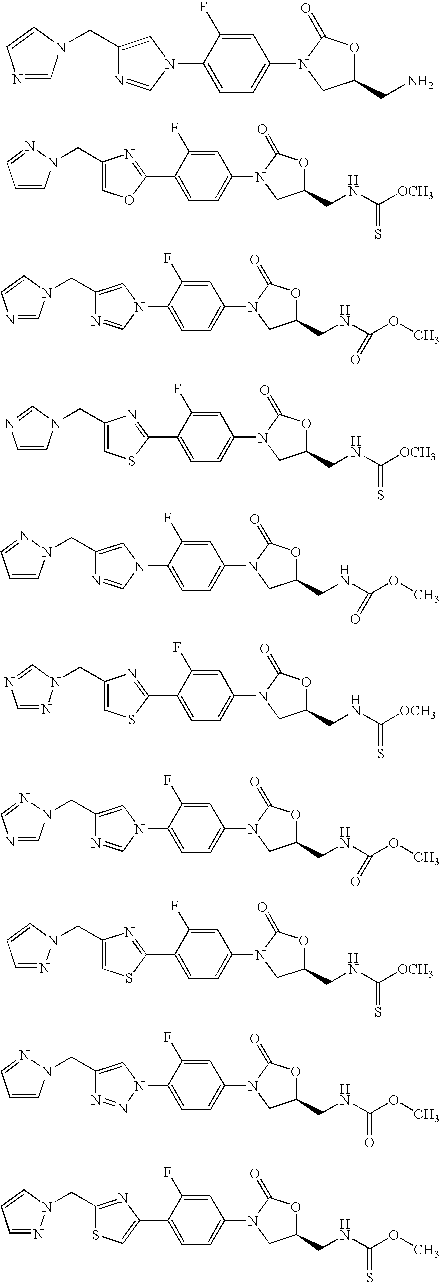

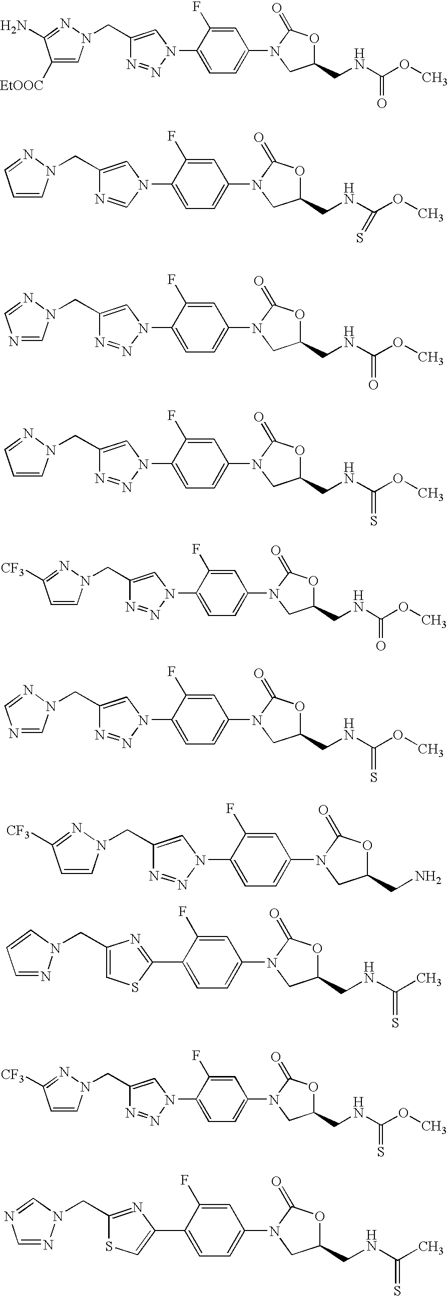

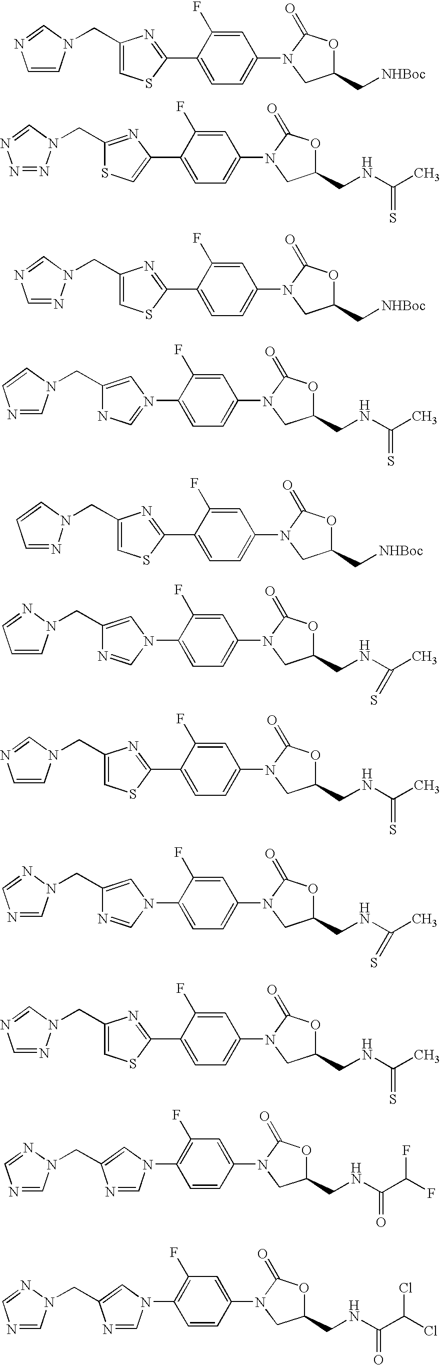

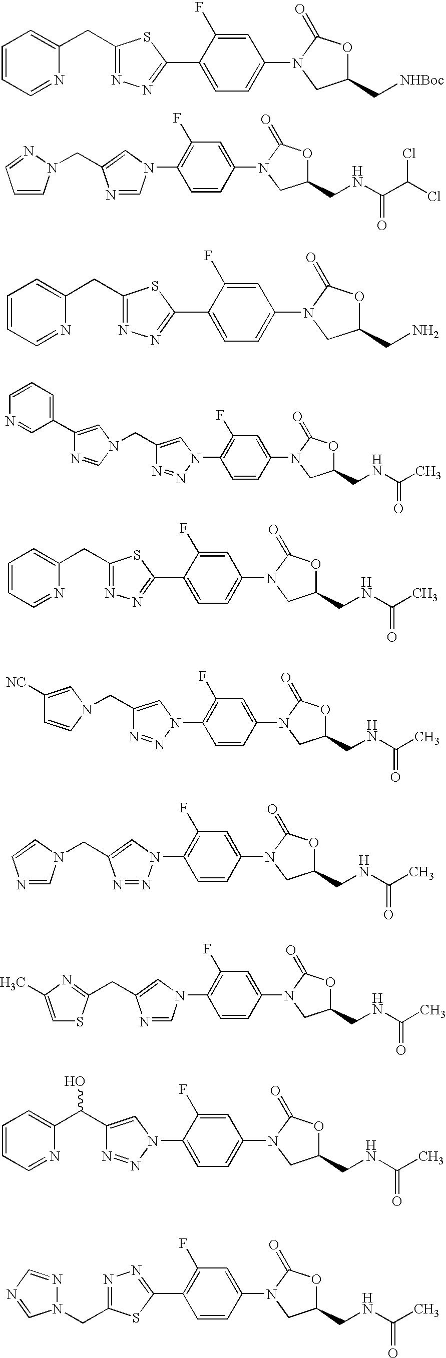

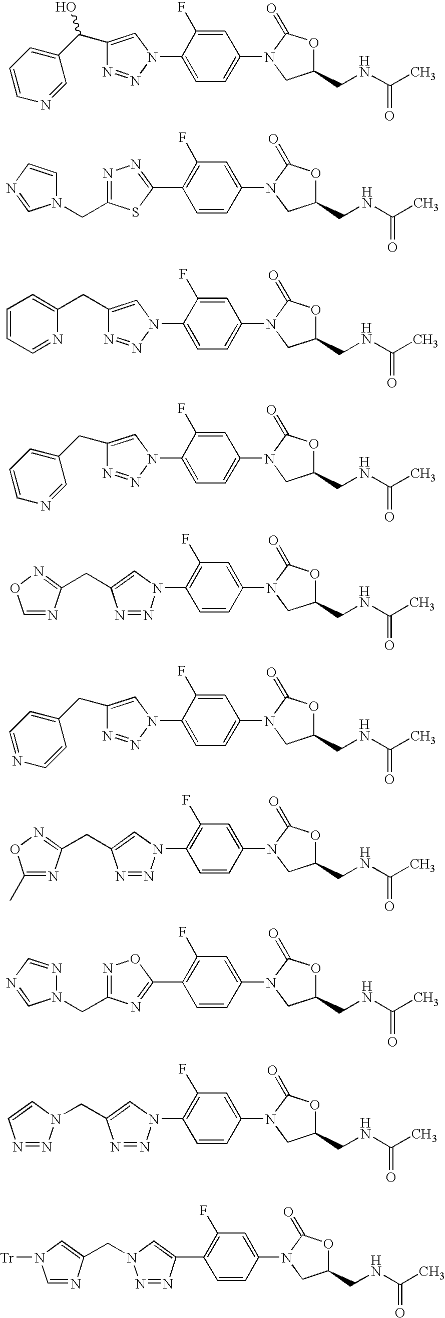

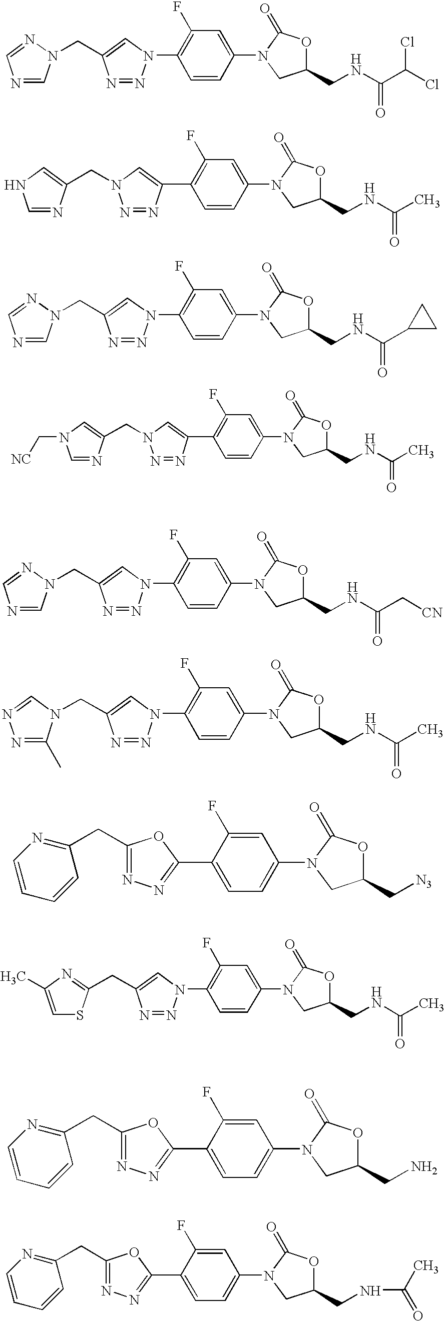

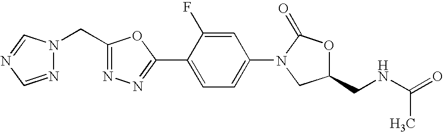

29. The compound as claimed in claim 1, wherein the compound is a salt chosen from Li, Na, K, Ca, Mg, Fe, Cu, Zn, Mn or Al, salts of organic bases, salts of natural amino acids, salts of guanidine, or salts of ammonium.

30. A pharmaceutical composition comprising a compound as claimed in claim 1, and one or more pharmaceutically acceptable excipients.

31. A method of producing antibacterial activity against pathogens in a subject, said method comprising administering to said subject an effective amount of a compound as claimed in claim 1.
